(19) United States
(12) Patent Application Publication
(10) Pub. No.: US 2005/0170385 A1
(43) Pub. Date: Aug. 4, 2005

(54) Artificial receptors including gradients

(75) Inventor: Robert E. Carlson, Minnetonka, MN (US)

Correspondence Address: MERCHANT & GOULD PC, P.O. BOX 2903, MINNEAPOLIS, MN, 55402-0903, US

(73) Assignee: RECEPTORS LLC, CHASKA, MN (US)

(21) Appl. No.: 11/004,593

(22) Filed: Dec. 2, 2004

Related U.S. Application Data
(63) Continuation-in-part of application No. 10/244,727, filed on Sep. 16, 2002.
(63) Continuation-in-part of application No. 10/812,850, filed on Mar. 29, 2004.
(63) Continuation-in-part of application No. 10/813,568, filed on Mar. 29, 2004.
(63) Continuation-in-part of application No. 10/813,612, filed on Mar. 29, 2004.
(63) Continuation-in-part of application No. 10/934,977, filed on Sep. 3, 2004.
(63) Continuation-in-part of application No. 10/934,977, filed on Sep. 3, 2004.
(63) Continuation-in-part of application No. 10/934,865, filed on Sep. 3, 2004.
(60) Provisional application No. 60/526,511, filed on Dec. 2, 2003.
(60) Provisional application No. 60/526,699, filed on Dec. 2, 2003.
(60) Provisional application No. 60/526,703, filed on Dec. 2, 2003.
(60) Provisional application No. 60/526,708, filed on Dec. 2, 2003.
(60) Provisional application No. 60/527,190, filed on Dec. 2, 2003.
(60) Provisional application No. 60/607,438, filed on Sep. 3, 2004.
(60) Provisional application No. 60/607,457, filed on Sep. 3, 2004.
(60) Provisional application No. 60/607,458, filed on Sep. 3, 2004.
(60) Provisional application No. 60/608,557, filed on Sep. 10, 2004.
(60) Provisional application No. 60/608,654, filed on Sep. 10, 2004.
(60) Provisional application No. 60/609,160, filed on Sep. 11, 2004.
(60) Provisional application No. 60/612,666, filed on Sep. 23, 2004.
(60) Provisional application No. 60/622,086, filed on Oct. 25, 2004.

(30) Foreign Application Priority Data
Feb. 19, 2003 (WO) WO 03/05328
Mar. 29, 2004 (WO) WO 04/09649
Sep. 3, 2004 (WO) WO 04/29050
Sep. 3, 2004 (WO) WO 04/29122

(51) Int. Cl. C12Q 1/68; G06F 19/00; G01N 33/48; G01N 33/50

(52) U.S. Cl. 435/6; 702/19

(57) ABSTRACT

The present invention relates to gradients of artificial receptors or building blocks, methods of making the gradients, and methods employing the gradients. The gradient can include one or more building blocks. The gradient can include change in any of a variety of characteristics of the artificial receptor or building block including change in the concentration of an artificial receptor or building block; change in the identity of an artificial receptor or building block; change in the topography of an artificial receptor or building block; change in the mode of binding of an artificial receptor or building block to the support; change in the lawn or lawn modifier; change in charge, volume, lipophilicity, or hydrophilicity of the artificial receptor or building block; or change in a molecular descriptors for the artificial receptor or building block.

### CROSS REFERENCE TO RELATED APPLICATIONS

The present application claims priority to U.S. Provisional Patent Application Nos. 60/527,190 filed Dec. 2, 2003, and 60/622,086, filed Oct. 25, 2004, each entitled “ARTIFICIAL RECEPTORS INCLUDING GRADIENTS”; is a continuation in part of copending U.S. patent application Ser. No. 10/244,727, filed Sep. 16, 2002, (which claims priority to U.S. Provisional Patent Application Nos. 60/360,980 filed Mar. 1, 2002; 60/362,600, filed Mar. 8, 2002; 60/375,655, filed Apr. 26, 2002; and 60/400,605, filed Aug. 2, 2002), Ser. No. 10/813,568, filed Mar. 29, 2004, and Application No. PCT/US03/05328, filed Feb. 19, 2003, each entitled “ARTIFICIAL RECEPTORS, BUILDING BLOCKS, AND METHODS”; is a continuation in part of U.S. patent application Ser. Nos. 10/812,850 and 10/813,612, and application No. PCT/US2004/009649, each filed Mar. 29, 2004 and each entitled “ARTIFICIAL RECEPTORS INCLUDING REVERSIBLY IMMOBILIZED BUILDING BLOCKS, THE BUILDING BLOCKS, AND METHODS”; is a continuation in part of U.S. patent application Ser. No. 10/934,977 and application No. PCT/US2004/29050, each filed Sep. 3, 2004 and each entitled “METHODS EMPLOYING COMBINATORIAL ARTIFICIAL RECEPTORS”; is a continuation in part of U.S. patent application Ser. No. 10/934,865 and application No. PCT/US2004/29122, each filed Sep. 3, 2004 and each entitled “BUILDING BLOCKS FOR ARTIFICIAL RECEPTORS”; and claims priority to U.S. Provisional Patent Application Nos. 60/526,511 60/526,699, 60/526,703, and 60/526,708 each filed Dec. 2, 2003; 60/607,438, 60/607,458, 60/607,457, each filed Sep. 3, 2004; 60/608,557 and 60/608,654, each filed Sep. 10, 2004; 60/609,160, filed Sep. 11, 2004; and 60/612,666, filed Sep. 23, 2004 and entitled “ARTIFICIAL RECEPTORS, BUILDING BLOCKS, AND METHODS”. Each of these patent applications is incorporated herein by reference in its entirety.

### FIELD OF THE INVENTION

The present invention relates to gradients of artificial receptors or building blocks, methods of making the gradients, and methods employing the gradients. The gradient can include one or more building blocks. The gradient can include change in any of a variety of characteristics of the artificial receptor or building block.

### BACKGROUND

The preparation of artificial receptors that bind ligands like proteins, peptides, carbohydrates, microbes, pollutants, pharmaceuticals, and the like with high sensitivity and specificity is an active area of research. None of the conventional approaches has been particularly successful; achieving only modest sensitivity and specificity mainly due to low binding affinity.

Antibodies, enzymes, and natural receptors generally have binding constants in the 10⁸-10¹² range, which results in both nanomolar sensitivity and targeted specificity. By contrast, conventional artificial receptors typically have binding constants of about 10³ to 10⁵, with the predictable result of millimolar sensitivity and limited specificity.

Several conventional approaches are being pursued in attempts to achieve highly sensitive and specific artificial receptors. These approaches include, for example, affinity isolation, molecular imprinting, and rational and/or combinatorial design and synthesis of synthetic or semi-synthetic receptors.

Such rational or combinatorial approaches have been limited by the relatively small number of receptors which are evaluated and/or by their reliance on a design strategy which focuses on only one building block, the homogeneous design strategy. Common combinatorial approaches form microarrays that include 10,000 or 100,000 distinct spots on a standard microscope slide. However, such conventional methods for combinatorial synthesis provide a single molecule per spot. Employing a single building block in each spot provides only a single possible receptor per spot. Synthesis of thousands of building blocks would be required to make thousands of possible receptors.

Further, these conventional approaches are hampered by the currently limited understanding of the principals which lead to efficient binding and the large number of possible structures for receptors, which makes such an approach problematic.

There remains a need for methods for detecting ligands and for detecting compounds that disrupt one or more binding interactions.

### SUMMARY

The present invention relates to gradients of artificial receptors or building blocks, methods of making the gradients, and methods employing the gradients. The gradient can include one or more building blocks. The gradient can include change in any of a variety of characteristics of the artificial receptor or building block including change in the concentration of an artificial receptor or building block; change in the identity of an artificial receptor or building block; change in the topography of an artificial receptor or building block; change in the mode of binding of an artificial receptor or building block to the support; change in the lawn or lawn modifier; change in charge, volume, lipophilicity, or hydrophilicity of the artificial receptor or building block; or change in a molecular descriptors for the artificial receptor or building block.

In an embodiment, the present invention relates to a building block gradient. The building block gradient can include a support. A portion of the support can include at least one building block. The building block being coupled to the support and the building block forming a gradient. In an embodiment, the building block gradient includes a surface. The surface includes a region including at least one building block. The building block being coupled to the support and the building block forming a gradient. The gradient can include a plurality of building blocks.

In an embodiment, the present invention relates to a method of making a building block gradient. The method can include forming a region on a solid support. The region can include at least one building block, the building block forming a gradient. The method also includes coupling the building block to the solid support in the region. The method can include coupling a plurality of building blocks to the solid support in the region.

In an embodiment, the present invention relates to a method of using a building block gradient. This method can include contacting a the building block gradient with a test ligand. and monitoring the gradient for binding of the test ligand. The test ligand can be a protein or proteome.

### BRIEF DESCRIPTION OF THE FIGURES

FIG. 1 schematically illustrates two dimensional representations of an embodiment of a receptor according to the present invention that employs 4 different building blocks to make a ligand binding site.

FIG. 2 schematically illustrates two and three dimensional representations of an embodiment of a molecular configuration of 4 building blocks, each building block including a recognition element, a framework, and a linker coupled to a support (immobilization/anchor).

FIG. 3 schematically illustrates an embodiment of the present methods and artificial receptors employing shuffling and exchanging building blocks.

FIG. 4 schematically illustrates a gradient extending in one direction along a support, such as a slide or plate.

FIG. 5 schematically illustrates gradients extending in two opposite directions along a support, such as a slide or plate.

FIG. 6 schematically illustrates multiple gradients extending in each of two opposite directions along a support, such as a slide or plate.

FIG. 7 schematically illustrates two gradients extending in different directions on a support, such as a slide or plate.

FIG. 8 schematically illustrates three gradients extending in different directions on a support, such as a slide or plate.

FIG. 9 schematically illustrates four gradients extending in different directions on a support, such as a slide or plate.

FIG. 10 also schematically illustrates four gradients extending in different directions on a support, such as a slide or plate.

FIG. 11 schematically illustrates a step gradient extending across a support, such as a slide or plate.

FIG. 12 schematically illustrates a plate including both step gradient A and a second gradient B.

FIG. 13 schematically illustrates an embodiment of a method for making and using a gradient for evaluating an analyte or mixture of analytes.

FIG. 14 schematically illustrates a false color fluorescence image of a labeled microarray according to an embodiment of the present invention.

FIG. 15 schematically illustrates a two dimensional plot of data obtained for candidate artificial receptors contacted with and/or binding phycoerythrin.

FIG. 16 schematically illustrates a three dimensional plot of data obtained for candidate artificial receptors contacted with and/or binding phycoerythrin.

FIG. 17 schematically illustrates a two dimensional plot of data obtained for candidate artificial receptors contacted with and/or binding a fluorescent derivative of ovalbumin.

FIG. 18 schematically illustrates a three dimensional plot of data obtained for candidate artificial receptors contacted with and/or binding a fluorescent derivative of ovalbumin.

FIG. 19 schematically illustrates a two dimensional plot of data obtained for candidate artificial receptors contacted with and/or binding a fluorescent derivative of bovine serum albumin.

FIG. 20 schematically illustrates a three dimensional plot of data obtained for candidate artificial receptors contacted with and/or binding a fluorescent derivative of bovine serum albumin.

FIG. 21 schematically illustrates a two dimensional plot of data obtained for candidate artificial receptors contacted with and/or binding an acetylated horseradish peroxidase.

FIG. 22 schematically illustrates a three dimensional plot of data obtained for candidate artificial receptors contacted with and/or binding an acetylated horseradish peroxidase.

FIG. 23 schematically illustrates a two dimensional plot of data obtained for candidate artificial receptors contacted with and/or binding a TCDD derivative of horseradish peroxidase.

FIG. 24 schematically illustrates a three dimensional plot of data obtained for candidate artificial receptors contacted with and/or binding a TCDD derivative of horseradish peroxidase.

FIG. 25 schematically illustrates a subset of the data illustrated in FIG. 16.

FIG. 26 schematically illustrates a subset of the data illustrated in FIG. 16.

FIG. 27 schematically illustrates a subset of the data illustrated in FIG. 16.

FIG. 28 schematically illustrates a correlation of binding data for phycoerythrin against logP for the building blocks making up the artificial receptor.

FIG. 29 schematically illustrates a correlation of binding data for phycoerythrin against logP for the building blocks making up the artificial receptor.

FIG. 30 schematically illustrates a two dimensional plot comparing data obtained for candidate artificial receptors contacted with and/or binding phycoerythrin to data obtained for candidate artificial receptors contacted with and/or binding a fluorescent derivative of bovine serum albumin.

FIGS. 31, 32, and 33 schematically illustrate subsets of data from FIGS. 16, 20, and 18, respectively, and demonstrate that the array of artificial receptors according to the present invention yields receptors distinguished between three analytes, phycoerythrin, bovine serum albumin, and ovalbumin.

FIG. 34 schematically illustrates a gray scale image of the fluorescence signal from a scan of a control plate which was prepared by washing off the building blocks with organic solvent before incubation with the test ligand.

FIG. 35 schematically illustrates a gray scale image of the fluorescence signal from a scan of an experimental plate which was incubated with 1.0 μg/ml Cholera Toxin B at 23° C.

FIG. 36 schematically illustrates a gray scale image of the fluorescence signal from a scan of an experimental plate which was incubated with 1.0 μg/ml Cholera Toxin B at 3° C.

FIG. 37 schematically illustrates a gray scale image of the fluorescence signal from a scan of an experimental plate which was incubated with 1.0 μg/ml Cholera Toxin B at 43° C.

FIGS. 38-40 schematically illustrate plots of the fluorescence signals obtained from the candidate artificial receptors illustrated in FIGS. 35-37.

FIG. 41 schematically illustrate plots of the fluorescence signals obtained from the combinations of building blocks employed in the present studies, when those building blocks are covalently linked to the support. Binding was conducted at 23° C.

FIG. 42 schematically illustrates the changes in fluorescence signal from individual combinations of covalently immobilized building blocks at 4° C., 23° C., or 44° C.

FIG. 43 schematically illustrates a graph of the changes in fluorescence signal from individual combinations of building blocks at 4° C., 23° C., or 44° C.

FIG. 44 schematically illustrates the data presented in FIG. 42 (lines marked A) and the data presented in FIG. 43 (lines marked B).

FIG. 45 schematically illustrates a graph of the fluorescence signal at 44° C. divided by the signal at 23° C. against the fluorescence signal obtained from binding at 23° C. for the artificial receptors with reversibly immobilized receptors.

FIG. 46 illustrates fluorescence signals produced by binding of cholera toxin to a microarray of the present candidate artificial receptors followed by washing with buffer in an experiment reported in Example 4.

FIG. 47 illustrates the fluorescence signals due to cholera toxin binding that were detected upon competition with GM1 OS (0.34 μM) in an experiment reported in Example 4.

FIG. 48 illustrates the ratio of the amount bound in the absence of GM1 OS to the amount bound in competition with GM1 OS (0.34 μM) in an experiment reported in Example 4.

FIG. 49 illustrates fluorescence signals produced by binding of cholera toxin to a microarray of the present candidate artificial receptors followed by washing with buffer in an experiment reported in Example 4 and for comparison with competition experiments using 5.1 μM GM1 OS.

FIG. 50 illustrates the fluorescence signals due to cholera toxin binding that were detected upon competition with GM1 OS (5.1 μM) in an experiment reported in Example 4.

FIG. 51 illustrates the ratio of the amount bound in the absence of GM1 OS to the amount bound in competition with GM1 OS (5.1 μM) in an experiment reported in Example 4.

FIG. 52 illustrates the fluorescence signals produced by binding of cholera toxin to the microarray of candidate artificial receptors alone and in competition with each of the three concentrations of GM1 in the experiment reported in Example 5.

FIG. 53 illustrates the ratio of the amount bound in the absence of GM1 OS to the amount bound upon competition with GM1 for the low concentration of GM1 employed in Example 5.

FIG. 54 illustrates the fluorescence signals produced by binding of cholera toxin to the microarray of candidate artificial receptors without pretreatment with GM 1 in the experiment reported in Example 6.

FIGS. 55-57 illustrate the fluorescence signals produced by binding of cholera toxin to the microarray of candidate artificial receptors with pretreatment with GM1 (100 μg/ml, 10 μg/ml, and 1 μg/ml GM1, respectively) in the experiment reported in Example 6.

FIG. 58 illustrates the ratio of the amount bound in the presence of 1 μg/ml GM1 to the amount bound in the absence of GM1 in the experiment reported in Example 6.

FIG. 59 presents an image obtained from a run of phycoerythrin over a step gradient of increasing concentrations of the building block TyrA₃B₃.

FIG. 60 shows increasing peaks of fluorescence for the 3^{rd}, 4^{th}, 5^{th}, and 6^{th} steps of the building block gradient.

FIG. 61 presents an image obtained from a run of phycoerythrin over a step gradient of increasing concentrations of the building block TyrA₄B₄.

FIG. 62 shows increasing peaks of fluorescence for the 5^{th} and 6^{th} steps of the building block gradient.

FIG. 63 presents an image obtained from a run of phycoerythrin over a step gradient of increasing concentrations of the building block TyrA₅B₅.

FIG. 64 illustrates that phycoerythrin did not bind to any step of the gradient of this building block, no peaks of fluorescence were obtained.

FIG. 65 presents an image obtained from a run of cholera toxin over a step gradient of increasing concentrations of the building block TyrA₃B₃.

FIG. 66 shows increasing peaks of fluorescence for the 2^{nd}, 3^{rd}, 4^{th}, 5^{th}, and 6^{th} steps of the building block gradient.

FIG. 67 presents an image obtained from a run of cholera toxin over a step gradient of increasing concentrations of the building block TyrA₄B₄.

FIG. 68 shows increasing peaks of fluorescence for at least the 2^{nd}, 3^{rd}, 4^{th}, 5^{th}, and 6^{th} steps of the building block gradient and possibly also for the first step.

FIG. 69 presents an image obtained from a run of cholera toxin over a step gradient of increasing concentrations of the building block TyrA₅B₅.

FIG. 70 illustrates that cholera toxin did not bind to any step of the gradient of this building block, no peaks of fluorescence were obtained.

FIG. 71 presents an image obtained from a run of cholera toxin over a step gradient of increasing concentrations of the building blocks TyrA₃B₃ and TyrA₄B₄ (in a 1:1 molar ratio).

FIG. 72 shows increasing peaks of fluorescence for the 3^{rd}, 4^{th}, 5^{th}, and 6^{th} steps of the building block gradient.

FIG. 73 presents an image obtained from a run of cholera toxin over a step gradient of increasing concentrations of the building blocks TyrA₃B₃ and TyrA₄B₄ (in a 1:1 molar ratio).

FIG. 74 shows peaks of fluorescence for at least the 6^{th}, 5^{th}, 4^{th}, 3^{th}, and 2^{nd} steps of the building block gradient.

FIG. 75 presents an image obtained from a run of a mixture of cholera toxin and phycoerythrin over a step gradient of increasing concentrations of the building blocks TyrA₃B₃ and TyrA₄B₄ (in a 1:1 molar ratio).

FIG. 76 shows fluorescence intensities obtained for cholera toxin (top line) and phycoerythrin (bottom line).

FIG. 77 presents an image obtained from a run of a mixture of cholera toxin and phycoerythrin over a step gradient of increasing concentrations of the building blocks TyrA₄B₄ and TyrA₄B₆ (in a 1:1 molar ratio).

FIG. 78 shows fluorescence intensities obtained for cholera toxin (top line) and phycoerythrin (bottom line).

FIG. 79 presents a fluorescence image from a run of cholera toxin flowed over a continuous gradient of increasing concentrations of the building blocks TyrA₃B₃ and TyrA₄B₄ (in a 1:1 molar ratio).

FIG. 80 illustrates increasing fluorescence, which indicates that the cholera toxin bound in increasing amounts to the higher concentration portions of the gradient.

### DETAILED DESCRIPTION

Definitions

As used herein, the term “peptide” refers to a compound including two or more amino acid residues joined by amide bond(s).

As used herein, the terms “polypeptide” and “protein” refer to a peptide including more than about 20 amino acid residues connected by peptide linkages.

As used herein, the term “proteome” refers to the expression profile of the proteins of an organism, tissue, organ, or cell. The proteome can be specific to a particular status (e.g., development, health, etc.) of the organism, tissue, organ, or cell.

Reversibly immobilizing building blocks on a support couples the building blocks to the support through a mechanism that allows the building blocks to be uncoupled from the support without destroying or unacceptably degrading the building block or the support. That is, immobilization can be reversed without destroying or unacceptably degrading the building block or the support. In an embodiment, immobilization can be reversed with only negligible or ineffective levels of degradation of the building block or the support. Reversible immobilization can employ readily reversible covalent bonding or noncovalent interactions. Suitable noncovalent interactions include interactions between ions, hydrogen bonding, van der Waals interactions, and the like. Readily reversible covalent bonding refers to covalent bonds that can be formed and broken under conditions that do not destroy or unacceptably degrade the building block or the support.

A combination of building blocks immobilized on, for example, a support can be a candidate artificial receptor, a lead artificial receptor, or a working artificial receptor. That is, a heterogeneous building block spot on a slide or a plurality of building blocks coated on a tube or well can be a candidate artificial receptor, a lead artificial receptor, or a working artificial receptor. A candidate artificial receptor can become a lead artificial receptor, which can become a working artificial receptor.

As used herein the phrase “candidate artificial receptor” refers to an immobilized combination of building blocks that can be tested to determine whether or not a particular test ligand binds to that combination. In an embodiment, the combination includes one or more reversibly immobilized building blocks. In an embodiment, the candidate artificial receptor can be a heterogeneous building block spot on a slide or a plurality of building blocks coated on a tube or well.

As used herein the phrase “lead artificial receptor” refers to an immobilized combination of building blocks that binds a test ligand at a predetermined concentration of test ligand, for example at 10, 1, 0.1, or 0.01 μg/ml, or at 1, 0.1, or 0.01 ng/ml. In an embodiment, the combination includes one or more reversibly immobilized building blocks. In an embodiment, the lead artificial receptor can be a heterogeneous building block spot on a slide or a plurality of building blocks coated on a tube or well.

As used herein the phrase “working artificial receptor” refers to a combination of building blocks that binds a test ligand with a selectivity and/or sensitivity effective for categorizing or identifying the test ligand. That is, binding to that combination of building blocks describes the test ligand as belonging to a category of test ligands or as being a particular test ligand. A working artificial receptor can, for example, bind the ligand at a concentration of, for example, 100, 10, 1, 0.1, 0.01, or 0.001 ng/ml. In an embodiment, the combination includes one or more reversibly immobilized building blocks. In an embodiment, the working artificial receptor can be a heterogeneous building block spot on a slide or a plurality of building blocks coated on a tube, well, slide, or other support or on a scaffold.

As used herein the phrase “working artificial receptor complex” refers to a plurality of artificial receptors, each a combination of building blocks, that binds a test ligand with a pattern of selectivity and/or sensitivity effective for categorizing or identifying the test ligand. That is, binding to the several receptors of the complex describes the test ligand as belonging to a category of test ligands or as being a particular test ligand. The individual receptors in the complex can each bind the ligand at different concentrations or with different affinities. For example, the individual receptors in the complex each bind the ligand at concentrations of 100, 10, 1, 0.1, 0.01 or 0.001 ng/ml. In an embodiment, the combination includes one or more reversibly immobilized building blocks. In an embodiment, the working artificial receptor complex can be a plurality of heterogeneous building block spots or regions on a slide; a plurality of wells, each coated with a different combination of building blocks; or a plurality of tubes, each coated with a different combination of building blocks.

As used herein, the phrase “significant number of candidate artificial receptors” refers to sufficient candidate artificial receptors to provide an opportunity to find a working artificial receptor, working artificial receptor complex, or lead artificial receptor. As few as about 100 to about 200 candidate artificial receptors can be a significant number for finding working artificial receptor complexes suitable for distinguishing two proteins (e.g., cholera toxin and phycoerythrin). In other embodiments, a significant number of candidate artificial receptors can include about 1,000 candidate artificial receptors, about 10,000 candidate artificial receptors, about 100,000 candidate artificial receptors, or more.

Although not limiting to the present invention, it is believed that the significant number of candidate artificial receptors required to provide an opportunity to find a working artificial receptor may be larger than the significant number required to find a working artificial receptor complex. Although not limiting to the present invention, it is believed that the significant number of candidate artificial receptors required to provide an opportunity to find a lead artificial receptor may be larger than the significant number required to find a working artificial receptor. Although not limiting to the present invention, it is believed that the significant number of candidate artificial receptors required to provide an opportunity to find a working artificial receptor for a test ligand with few features may be more than for a test ligand with many features.

As used herein, the term “building block” refers to a molecular component of an artificial receptor including portions that can be envisioned as or that include one or more linkers, one or more frameworks, and one or more recognition elements. In an embodiment, the building block includes a linker, a framework, and one or more recognition elements. In an embodiment, the linker includes a moiety suitable for reversibly immobilizing the building block, for example, on a support, surface or lawn. The building block interacts with the ligand.

As used herein, the term “linker” refers to a portion of or functional group on a building block that can be employed to or that does (e.g., reversibly) couple the building block to a support, for example, through covalent link, ionic interaction, electrostatic interaction, or hydrophobic interaction.

As used herein, the term “framework” refers to a portion of a building block including the linker or to which the linker is coupled and to which one or more recognition elements are coupled.

As used herein, the term “recognition element” refers to a portion of a building block coupled to the framework but not covalently coupled to the support. Although not limiting to the present invention, the recognition element can provide or form one or more groups, surfaces, or spaces for interacting with the ligand.

As used herein, the phrase “plurality of building blocks” refers to two or more building blocks of different structure in a mixture, in a kit, or on a support or scaffold. Each building block has a particular structure, and use of building blocks in the plural, or of a plurality of building blocks, refers to more than one of these particular structures. Building blocks or plurality of building blocks does not refer to a plurality of molecules each having the same structure.

As used herein, the phrase “combination of building blocks” refers to a plurality of building blocks that together are in a spot, region, or a candidate, lead, or working artificial receptor. A combination of building blocks can be a subset of a set of building blocks. For example, a combination of building blocks can be one of the possible combinations of 2, 3, 4, 5, or 6 building blocks from a set of N (e.g., N=10-200) building blocks.

As used herein, the phrases “homogenous immobilized building block” and “homogenous immobilized building blocks” refer to a support or spot having immobilized on or within it only a single building block.

As used herein, the phrase “activated building block” refers to a building block activated to make it ready to form a covalent bond to a functional group, for example, on a support. A building block including a carboxyl group can be converted to a building block including an activated ester group, which is an activated building block. An activated building block including an activated ester group can react, for example, with an amine to form a covalent bond.

As used herein, the term “naïve” used with respect to one or more building blocks refers to a building block that has not previously been determined or known to bind to a test ligand of interest. For example, the recognition element(s) on a naïve building block has not previously been determined or known to bind to a test ligand of interest. A building block that is or includes a known ligand (e.g., GM1) for a particular protein (test ligand) of interest (e.g., cholera toxin) is not naïve with respect to that protein (test ligand).

As used herein, the term “immobilized” used with respect to building blocks coupled to a support refers to building blocks being stably oriented on the support so that they do not migrate on the support or release from the support. Building blocks can be immobilized by covalent coupling, by ionic interactions, by electrostatic interactions, such as ion pairing, or by hydrophobic interactions, such as van der Waals interactions.

As used herein a “region” of a support, tube, well, or surface refers to a contiguous portion of the support, tube, well, or surface. Building blocks coupled to a region can refer to building blocks in proximity to one another in that region.

As used herein, a “bulky” group on a molecule is larger than a moiety including 7 or 8 carbon atoms.

As used herein, a “small” group on a molecule is hydrogen, methyl, or another group smaller than a moiety including 4 carbon atoms.

As used herein, the term “lawn” refers to a layer, spot, or region of functional groups on a support, for example, at a density sufficient to place coupled building blocks in proximity to one another. The functional groups can include groups capable of forming covalent, ionic, electrostatic, or hydrophobic interactions with building blocks.

As used herein, the term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₁₂ for straight chain, C₁-C₆ for branched chain). Likewise, cycloalkyls can have from 3-10 carbon atoms in their ring structure, for example, 5, 6 or 7 carbons in the ring structure.

The term “alkyl” as used herein refers to both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aryl alkyl, or an aromatic or heteroaromatic moiety. The moieties substituted on the hydrocarbon chain can themselves be substituted, if appropriate. For example, the substituents of a substituted alkyl can include substituted and unsubstituted forms of the groups listed above.

The phrase “aryl alkyl”, as used herein, refers to an alkyl group substituted with an aryl group (e.g., an aromatic or heteroaromatic group).

As used herein, the terms “alkenyl” and “alkynyl” refer to unsaturated aliphatic groups analogous in length and optional substitution to the alkyls groups described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents such as those described above for alkyl groups. The term “aryl” also includes polycyclic ring systems having two or more cyclic rings in which two or more carbons are common to two adjoining rings (the rings are “fused rings”) wherein at least one of the rings is aromatic, e.g., the other cyclic ring(s) can be cycloalkyls, cycloalkenyls, cycloalkynyls, aryls and/or heterocyclyls.

As used herein, the terms “heterocycle” or “heterocyclic group” refer to 3- to 12-membered ring structures, e.g., 3- to 7-membered rings, whose ring structures include one to four heteroatoms. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents such as those described for alkyl groups.

As used herein, the term “heteroatom” as used herein means an atom of any element other than carbon or hydrogen, such as nitrogen, oxygen, sulfur and phosphorous.

Overview of the Artificial Receptor

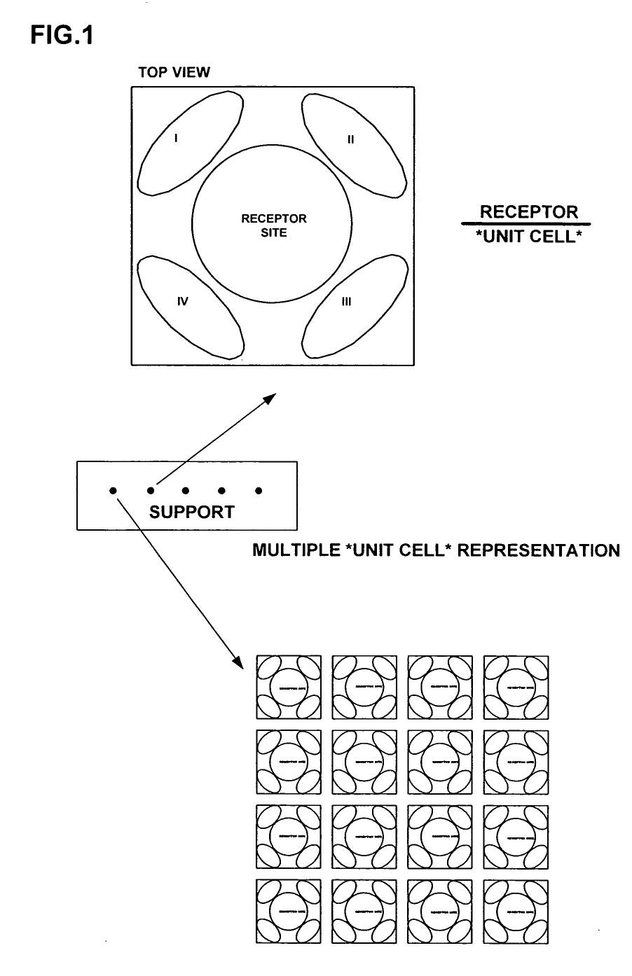

FIG. 1 schematically illustrates an embodiment employing 4 distinct building blocks in a spot on a microarray to make a ligand binding site. This figure illustrates a group of 4 building blocks at the corners of a square forming a unit cell. A group of four building blocks can be envisioned as the vertices on any quadrilateral. FIG. 1 illustrates that spots or regions of building blocks can be envisioned as multiple unit cells, in this illustration square unit cells. Groups of unit cells of four building blocks in the shape of other quadrilaterals can also be formed on a support.

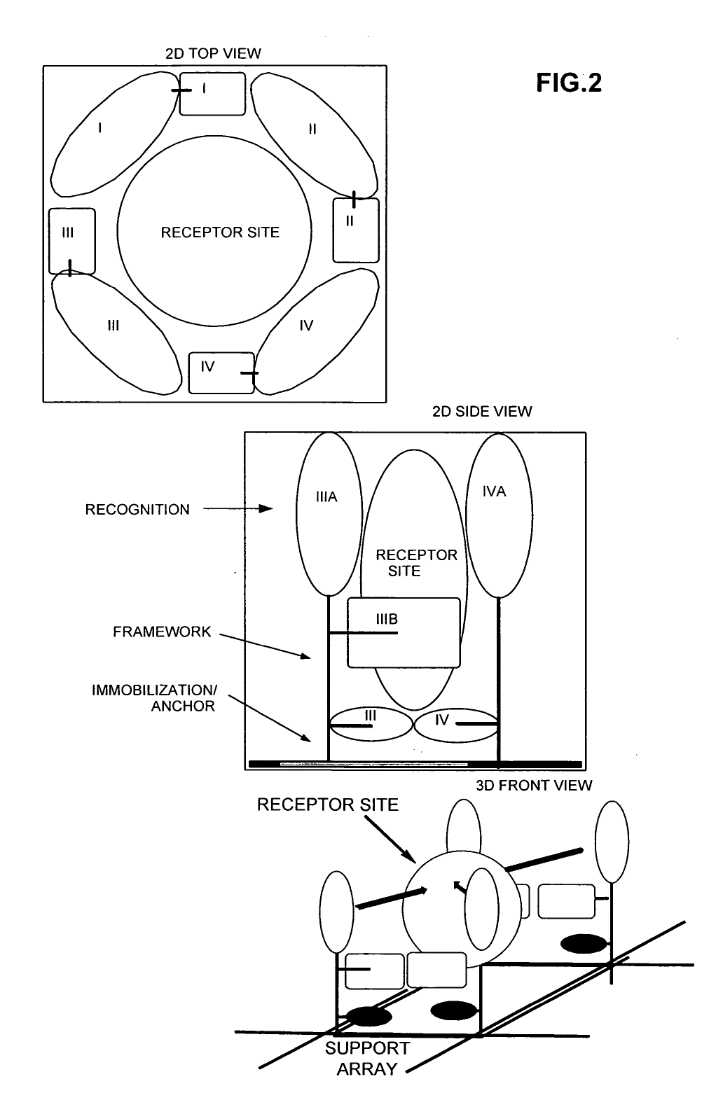

Each immobilized building block molecule can provide one or more “arms” extending from a “framework” and each can include groups that interact with a ligand or with portions of another immobilized building block. FIG. 2 illustrates that combinations of four building blocks, each including a framework with two arms (called “recognition elements”), provides a molecular configuration of building blocks that form a site for binding a ligand. Such a site formed by building blocks such as those exemplified below can bind a small molecule, such as a drug, metabolite, pollutant, or the like, and/or can bind a larger ligand such as a macromolecule or microbe.

The present artificial receptors can include building blocks reversibly immobilized on a support or surface. Reversing immobilization of the building blocks can allow movement of building blocks to a different location on the support or surface, or exchange of building blocks onto and off of the surface. For example, the combinations of building blocks can bind a ligand when reversibly coupled to or immobilized on the support. Reversing the coupling or immobilization of the building blocks provides opportunity for rearranging the building blocks, which can improve binding of the ligand. Further, the present invention can allow for adding additional or different building blocks, which can further improve binding of a ligand.

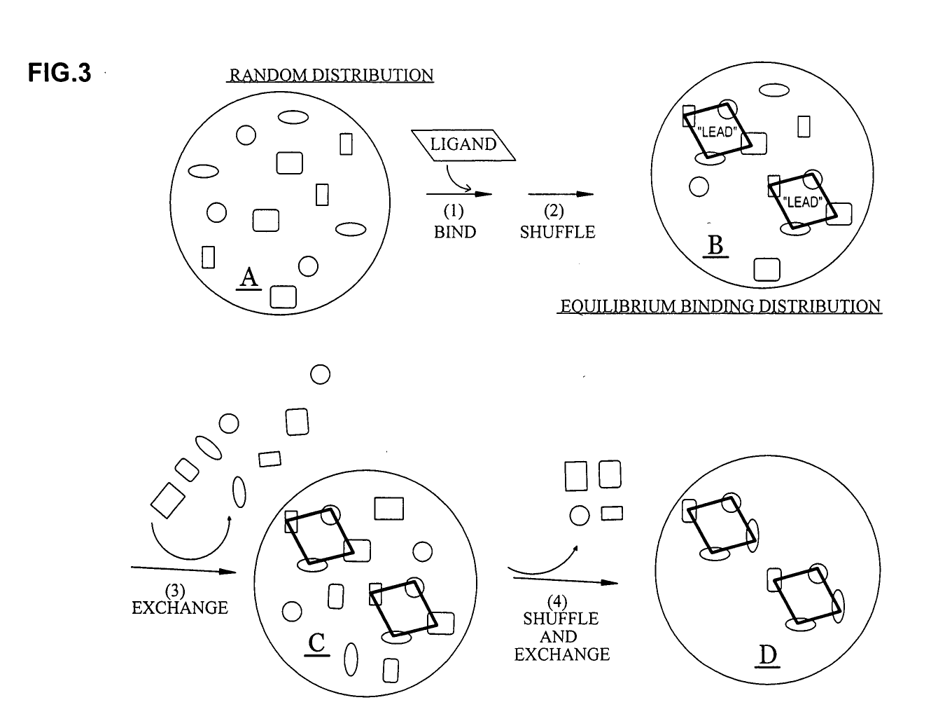

FIG. 3 schematically illustrates an embodiment employing an initial artificial receptor surface (A) with four different building blocks on the surface, which are represented by shaded shapes. This initial artificial receptor surface (A) undergoes (1) binding of a ligand to an artificial receptor and (2) shuffling the building blocks on the receptor surface to yield a lead artificial receptor (B). Shuffling refers to reversing the coupling or immobilization of the building blocks and allowing their rearrangement on the receptor surface. After forming a lead artificial receptor, additional building blocks can be (3) exchanged onto and/or off of the receptor surface (C). Exchanging refers to building blocks leaving the surface and entering a solution contacting the surface and/or building blocks leaving a solution contacting the surface and becoming part of the artificial receptor. The additional building blocks can be selected for structural diversity (e.g., randomly) or selected based on the structure of the building blocks in the lead artificial receptor to provide additional avenues for improving binding. The original and additional building blocks can then be (4) shuffled and exchanged to provide higher affinity artificial receptors on the surface (D).

Gradients of Artificial Receptors and Building Blocks

The present invention includes artificial receptors, building blocks, or combinations of building blocks configured as a gradient on a support. The gradient can be made up of change in the concentration (e.g., density) of an artificial receptor or building block. The gradient can be made up of change in the identity (e.g., structure) of an artificial receptor or building block. The gradient can be made up of change in the topography (e.g., size, shape, or flexibility) of an artificial receptors or building blocks. The gradient can be made up of change in the mode of binding (e.g., irreversible or reversible) of an artificial receptor or building block to the support. The gradient can be made up of changes in the lawn or lawn modifier. The gradient can be any of a variety of types of gradients, such as step, continuous, or the like.

One or more of building blocks according to the present invention can be immobilized on a support in regions. These regions can include one or more of the building blocks at different concentrations in different sub-regions. For example, one or more of the building blocks can be in different concentrations in bands on or across the region. For example, one or more of the building blocks can be in a gradient from zero or low concentration at one side (e.g., edge or corner) of the region to higher concentration at the opposite side (e.g., edge or corner) of the region. These regions can include distinct building blocks or combinations of building blocks in different sub-regions. For example, one or more of the building blocks can be in one sub-region but not another. For example, one or more of the building blocks can be at a first concentration in one sub-region and at a another (e.g., second) concentration in another sub-region. For example, one or more building blocks can be in each sub-region. For example, one or more building blocks can be in only a subset of sub-regions.

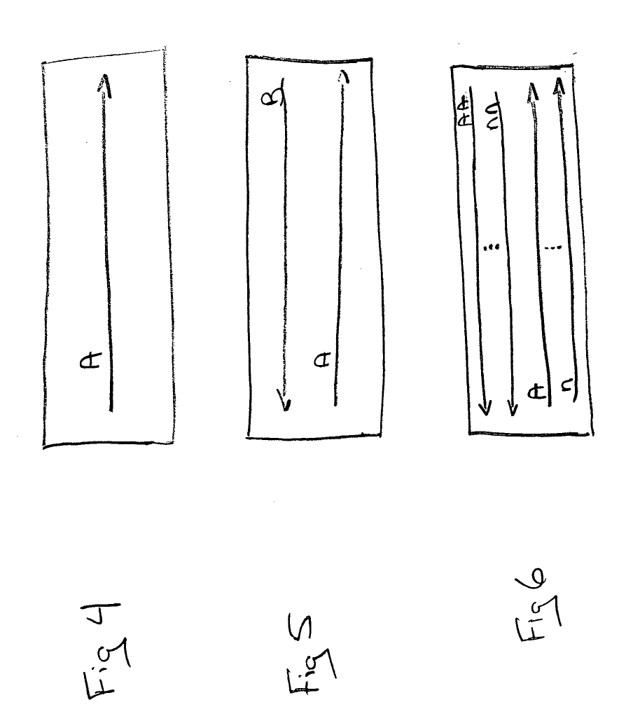

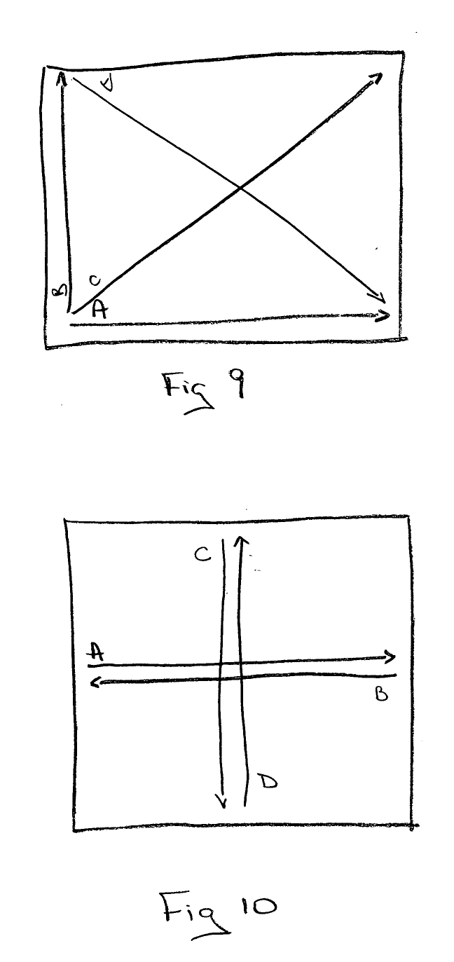

FIGS. 4 through 9 illustrate embodiments of supports or regions including gradients of artificial receptors or building blocks. FIGS. 4 through 6 generally relate to the number and direction of gradients in a single dimension (i.e., along a line) on a support. FIGS. 7 through 9 generally relate to the orientation of gradients in two dimensions on a support. Of course, each gradient with an orientation shown in FIGS. 7 through 9 can have the variation in number and direction illustrated in FIGS. 4 through 6. Further, although the gradients are illustrated as generally straight lines, a gradient according to the present invention can vary along any of a variety of trajectories. The change that occurs along the gradient can occur as a linear or other function of distance traveled from the beginning of or from a point along the gradient. For example, concentration can change as a linear or exponential function of distance from the beginning of the gradient.

FIG. 4 schematically illustrates a gradient extending in one direction along a support, such as a slide or plate. As described above, such a gradient can be made up of at least one of: change in the concentration (e.g., density) of an artificial receptor or building block; change in the identity (e.g., structure) of an artificial receptor or building block; change in the topography (e.g., size, shape, or flexibility) of an artificial receptor or building block; change in the mode of binding (e.g., irreversible or reversible) of an artificial receptor or building block to the support; and changes in the lawn or lawn modifier. The gradient can provide or be made up of changes in at least one of a variety of characteristics of the artificial receptor or building block, such as charge, volume, lipophilicity, hydrophilicity. The gradient can be described by or made up of changes in at least one of a variety of molecular descriptors for the artificial receptor or building block.

FIG. 5 schematically illustrates gradients extending in two opposite directions along a support, such as a slide or plate. Gradient A can be as described above with respect to FIG. 4. Gradient B can be made up of any of the changes in characteristics described above with respect to FIG. 4. Gradient B will generally not be selected to be the same gradient as Gradient A but in an opposite direction, which would just result in one gradient canceling the other and produce a generally uniform receptor surface. In an embodiment, gradient B can be selected to be of a different character than gradient A.

For example, if gradient A is made up of change in the concentration (e.g., density) of a first artificial receptor or building block, then gradient B can be made up of at least one of: change in the concentration (e.g., density) of a second artificial receptor or building block; change in the identity (e.g., structure) of an artificial receptor or building block; change in the topography (e.g., size, shape, or flexibility) of an artificial receptor or building block; change in the mode of binding (e.g., irreversible or reversible) of an artificial receptor or building block to the support; and change in the lawn or lawn modifier.

For example, if gradient A is made up of change in the identity (e.g., structure) of a first artificial receptor or building block, then gradient B can be made up of at least one of: change in the concentration (e.g., density) of an artificial receptor or building block; change in the identity (e.g., structure) of a second artificial receptor or building block; change in the topography (e.g., size, shape, or flexibility) of an artificial receptor or building block; change in the mode of binding (e.g., irreversible or reversible) of an artificial receptor or building block to the support; and change in the lawn or lawn modifier.

For example, if gradient A is made up of change in the topography (e.g., size, shape, or flexibility) of a first artificial receptor or building block, then gradient B can be made up of at least one of: change in the concentration (e.g., density) of an artificial receptor or building block; change in the identity (e.g., structure) of an artificial receptor or building block; change in the topography (e.g., size, shape, or flexibility) of a second artificial receptor or building block; change in the mode of binding (e.g., irreversible or reversible) of an artificial receptor or building block to the support; and change in the lawn or lawn modifier.

For example, if gradient A is made up of change in the mode of binding (e.g., irreversible or reversible) of a first artificial receptor or building block to the support, then gradient B can be made up of at least one of: change in the concentration (e.g., density) of an artificial receptor or building block; change in the identity (e.g., structure) of an artificial receptor or building block; change in the topography (e.g., size, shape, or flexibility) of a second artificial receptor or building block; change in the mode of binding (e.g., irreversible or reversible) of a second artificial receptor or building block to the support; and change in the lawn or lawn modifier.

For example, if gradient A is made up of a first change in the lawn or lawn modifier, then gradient B can be made up of at least one of: change in the concentration (e.g., density) of an artificial receptor or building block; change in the identity (e.g., structure) of an artificial receptor or building block; change in the topography (e.g., size, shape, or flexibility) of a second artificial receptor or building block; change in the mode of binding (e.g., irreversible or reversible) of a second artificial receptor or building block to the support; and a second change in the lawn or lawn modifier.

For example, if gradient A is made up of a first change in charge of the artificial receptor or building block, then gradient B can be made up of at least one of: a second change in charge of the artificial receptor or building block, a change in volume of the artificial receptor or building block, a change in lipophilicity of the artificial receptor or building block, or a change in hydrophilicity of the artificial receptor or building block.

For example, if gradient A is made up of a first change in volume of the artificial receptor or building block, then gradient B can be made up of at least one of: a change in charge of the artificial receptor or building block, a second change in volume of the artificial receptor or building block, a change in lipophilicity of the artificial receptor or building block, or a change in hydrophilicity of the artificial receptor or building block.

For example, if gradient A is made up of a first change in lipophilicity of the artificial receptor or building block, then gradient B can be made up of at least one of: a change in charge of the artificial receptor or building block, a change in volume of the artificial receptor or building block, a second change in lipophilicity of the artificial receptor or building block, or a change in hydrophilicity of the artificial receptor or building block.

For example, if gradient A is made up of a first change in hydrophilicity of the artificial receptor or building block, then gradient B can be made up of at least one of: a change in charge of the artificial receptor or building block, a change in volume of the artificial receptor or building block, a change in lipophilicity of the artificial receptor or building block, or a second change in hydrophilicity of the artificial receptor or building block.

For example, if gradient A is made up of change in a first molecular descriptor for the artificial receptor or building block, then gradient B can be made up of change in a second molecular descriptor for the artificial receptor or building block.

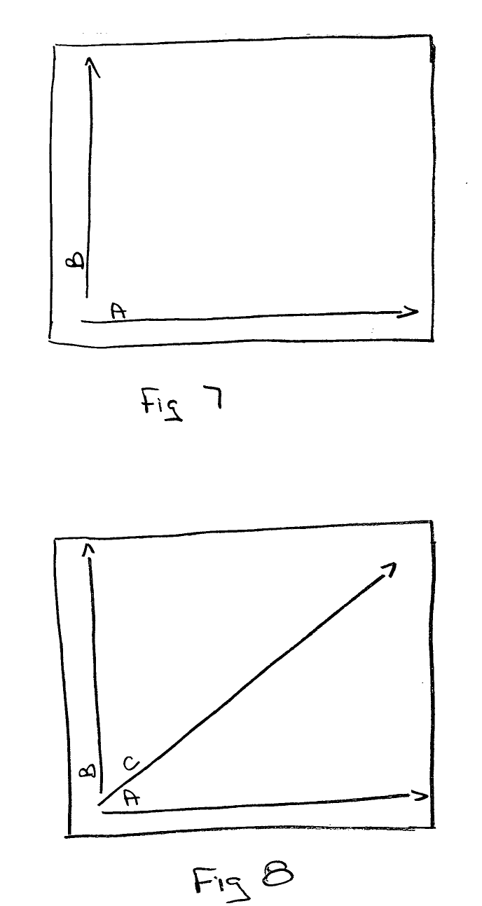

FIG. 6 schematically illustrates multiple gradients extending in each of two opposite directions along a support, such as a slide or plate. Each of the multiple gradients can be as described above with respect to FIGS. 4 and 5. A first gradient illustrated in FIG. 6 can relate to a second gradient in the ways described above for the relationships of gradients A and B. For example, gradient AA can relate to gradient A as gradient B in FIG. 5 relates to gradient A in FIG. 5. Gradient n can relate to gradient A as gradient B in FIG. 5 relates to gradient A. Generally gradient n will not be the same as gradient A, as this would just result in an additive increase in gradient A. In an embodiment, gradient B can be selected to be of a different character than gradient A. AA . . . nn and A . . . n as used in FIG. 6 each describe a plurality gradients.

FIG. 7 schematically illustrates two gradients extending in different directions on a support, such as a slide or plate. Each of gradients A and B can be as described above with respect to FIGS. 4 and 5. Typically gradient A will be different in character than gradient B, as described above with respect to FIG. 5. The examples of different characteristics for gradients A and B as described above can apply to gradients A and B in FIG. 7, which are oriented in different but not necessarily opposite directions. Either or both of gradients A and B can be made up of multiple gradients as described above with respect to FIG. 6. For example, gradient A in FIG. 7 can be or include gradients A through n. For example, gradient B in FIG. 7 can be or include gradients AA through nn. The orientation of gradients of artificial receptors or building blocks shown in FIG. 7 can be viewed as providing a support for a process analogous to 2-dimensional electrophoresis.

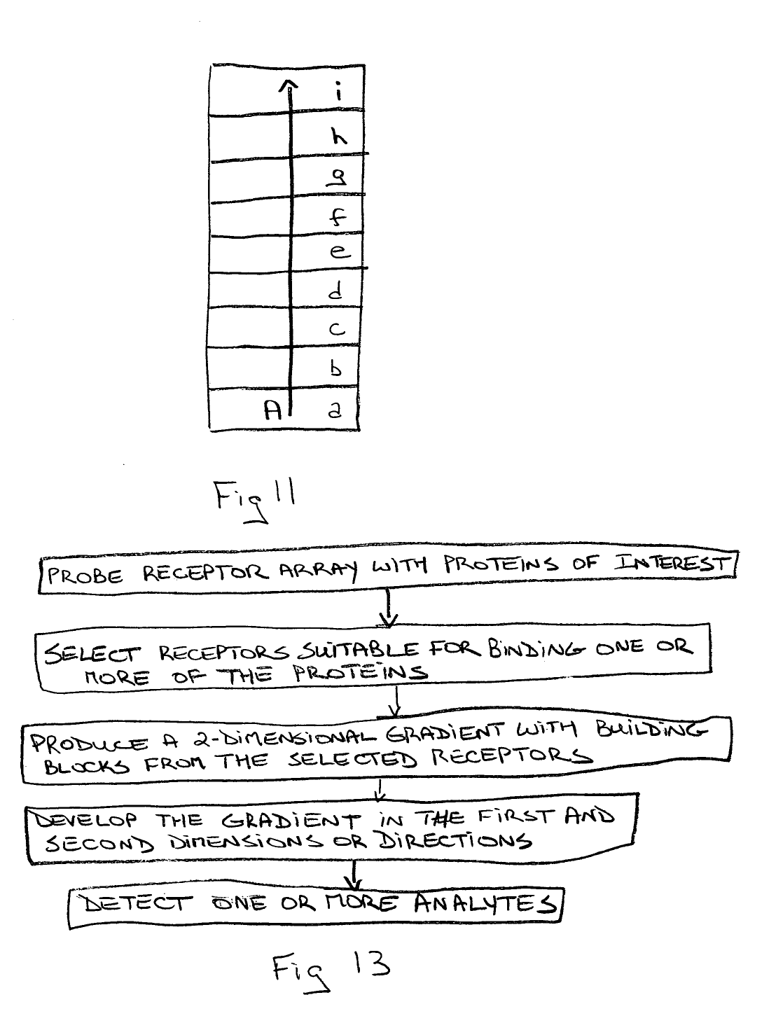

FIG. 8 schematically illustrates three gradients extending in different directions on a support, such as a slide or plate. Each of gradients A, B and C can be as described above with respect to FIGS. 4 and 5. The examples of different characteristics for gradients A and B as described above in the description of embodiments of FIG. 5 can apply to gradients A, B, and C in FIG. 8, which are oriented in different but not necessarily opposite directions. Typically gradient A will be different in character than gradient B, gradient A will be different in character than gradient C, and gradient B will be different in character than gradient C, as described above for gradients A and B in FIG. 5. One or more of gradients A, B or C can be made up of multiple gradients as described above with respect to FIG. 6. For example, gradient A, B, or C in FIG. 8 can be or include gradients A through n or gradients AA through nn. The orientation of gradients of artificial receptors or building blocks shown in FIG. 8 can be viewed as providing a support for a process analogous to 2-dimensional electrophoresis but including gradients of three features providing characterization of analytes in three directions.

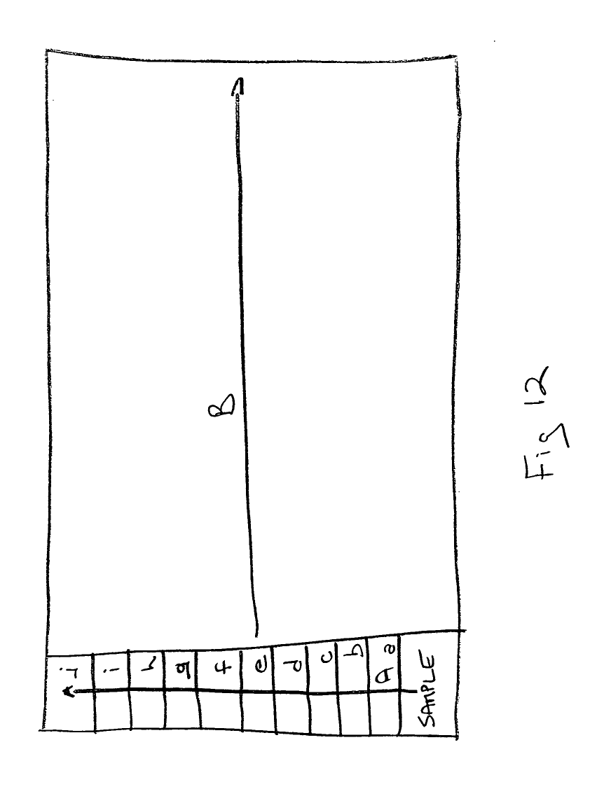

FIG. 9 schematically illustrates four gradients extending in different directions on a support, such as a slide or plate. Each of gradients A, B, C and D can be as described above with respect to FIGS. 4 and 5. The examples of different characteristics for gradients A and B as described above in the description of embodiments of FIG. 5 can apply to gradients A, B, C, and D in FIG. 9, which are oriented in different but not necessarily opposite directions. Typically gradient A will be different in character than gradient B, gradient A will be different in character than gradient C, gradient A will be different in character than gradient D, gradient B will be different in character than gradient C, gradient B will be different in character than gradient D, and gradient C will be different in character than gradient D, as described above for gradients A and B in FIG. 5. One or more of gradients A, B, C or D can be made up of multiple gradients as described above with respect to FIG. 6. For example, gradient A, B, C, or D in FIG. 9 can be or include gradients A through n or gradients AA through nn. The orientation of gradients of artificial receptors or building blocks shown in FIG. 9 can be viewed as providing a support for a process analogous to 2-dimensional electrophoresis but including gradients of four features providing characterization of analytes in four directions.

FIG. 10 also schematically illustrates four gradients extending in different directions on a support, such as a slide or plate. Each of gradients A, B, C and D can be as described above with respect to FIG. 9. In this embodiment, gradients A and B are oriented in opposite directions as are gradients C and D. In an embodiment, FIG. 10 can schematically illustrate a rectangular plate with 1, 2, 3, 4, or more building blocks in a gradient from as many as each of the edges of the plate to the opposite edge.

In an embodiment, individual or combinations of building blocks according to the present invention can be immobilized on a support in regions. Each region can include one or more building blocks each at a single concentration. The concentration can differ from region to region. In an embodiment, a concentration of building block can increase as the regions become more distant from an edge or origin. The regions can be adjacent to one another or separated from one another. The area between separated building blocks can include, for example, lawn or modified lawn. Such a gradient of building blocks can be envisioned as a region including one or more of the building blocks at different concentrations in different sub-regions. For example, one or more of the building blocks can be in different concentrations in bands on or across the region. For example, the one or more building blocks can be at zero or low concentration at one edge of the region and at a higher concentration at the opposite edge of the region.

FIG. 11 schematically illustrates a step gradient extending across a support, such as a slide or plate. Step gradient A includes steps a-i. The steps can be contiguous or separated by lawn or modified lawn. Gradient A can be as described with respect to FIG. 4 above. Step gradients can also be configured as described above for any one of FIGS. 5-10.

FIG. 12 schematically illustrates a plate including both step gradient A and a second gradient B. Step gradient A includes steps a-j. The steps can be contiguous or separated by lawn or modified lawn. Gradient B can be a step gradient, a continuous gradient, or any other type of gradient. Gradients A and B can be independently as described with respect to FIG. 4 above. Gradients A and B can also be independently configured as described above for any one of FIGS. 5-10. In an embodiment, the plate of FIG. 12 can include different building blocks or different concentrations of building block(s) in bands along, for example, one edge of a region. The second dimension of the region can be characterized by a gradient of concentrations or identity of building blocks.

Methods of Making a Surface-Bound Molecular Gradient of Building Blocks

The present invention relates to a method of making a surface-bound molecular gradient of building blocks. In an embodiment, this method includes preparing a spot or region on a support, the spot or region including a plurality of building blocks immobilized on the support in a gradient. The method can include forming a plurality of spots or regions on a solid support, each spot or region including a plurality of immobilized building blocks, in which the density, concentration, and number of building blocks varies across the support. In an embodiment, the concentration of building blocks varies across a region on a support or between a plurality of spots or regions on a solid support.

In an embodiment, a gradient of building blocks is formed on a support through the kinetics of the immobilization reactions described herein. The kinetics of immobilization can be influenced by a number of variables including, but not limited to: varying the concentration of the building blocks; varying the concentration of reactive sites within or between spots or regions on the surface, support, or lawn; and varying the time of reaction. Additional factors, such as temperature, pH, solvation, and catalysis can also be used to influence the immobilization conditions (e.g., rate of immobilization) and density of immobilized building block or lawn modifier.

In an embodiment, a method can be employed to produce a solid support having on its surface a plurality of regions or spots, each region or spot including a different concentration or identity of building blocks and forming a gradient across the solid support. In an embodiment, the solid support is exposed at a controlled rate to the building blocks. In an embodiment, the solid support has a lawn of reactive groups (e.g., amines) for the immobilization of building blocks.

In an embodiment, the solid support is placed non-horizontally (e.g., standing at an angle greater than 0°, typically between about 30° to 90°) in a container so that the support stands on one end against a wall of the container (e.g., beaker). The composition (e.g., solution) containing one or more building blocks is delivered into the container at a predetermined rate. For example, the solution can be introduced using a burette or pump. In an embodiment, the identity of the building blocks in the composition being delivered changes with time. In an embodiment, the concentration of building blocks in the composition being delivered changes with time. When the solution reaches the top of the solid support, the flow of solution is stopped and the solid support is subsequently removed from contact with the solution. In an embodiment, the solid support is rinsed to remove any remaining free building blocks. In an embodiment, quenching or termination agents are optionally applied to the solid support to limit further immobilization.

In an embodiment, a portion of the solid support near the bottom of the container has the longest time to react with the building block solution, and therefore has the highest concentration or density of immobilized building blocks. The portion of the solid support near the top of the solid support will have the least time to react, and therefore has a lower concentration or density of immobilized building blocks. The time of solution exposure in combination with the rate of the immobilization reaction determines the concentration or density of building blocks immobilized within regions or spots across the solid support.

In an embodiment, a constant flow of building block solution creates a linear density gradient across the solid support. In an embodiment, the flow rate may be changed thereby altering the slope of the gradient. In an embodiment, exponential gradients are created by varying the flow rate exponentially in time, or by simply using, for example, an Erlenmeyer flask to contain the support in the composition. An Erlenmeyer flask is narrower towards the top, such that the solution contacts the solid support at times that decrease exponentially from bottom to top. In an embodiment, turning the support, for example by either or 90° or 180°, with respect to the first gradient and repeating the procedure creates a 2-dimensional or multi-directional gradient.

Any of a variety of known methods can be employed to produce a gradient of building blocks across a region on a support, such as the surface of a plate or slide. For example, one or more building blocks can be eluted or diffused across the region to form a gradient. For example, one or more of the building blocks can be applied using a moving screen to form a gradient. For example, one or more of the building blocks can be poured into the center of the region and eluted or diffused across the region to form a gradient. In an embodiment, the solution of building blocks can be dispersed across a solid substrate by liquid or vapor phase diffusion. In an embodiment, building blocks can be distributed across a solid surface by diffusion through a matrix.

In an embodiment, a surface-bound molecular gradient can be made utilizing an energy-catalyzed (e.g., irradiation-catalyzed or initiated) chemistry to immobilize building blocks on the surface, support, or lawn. In an embodiment, the surface can be exposed to free building blocks (e.g., in solution). The surface can then be exposed to an energy source to catalyze the immobilization reaction. In an embodiment, either the solution of free building blocks or surface comprises an energy-activated reactant (e.g., visible light, UV initiator).

In an embodiment, the energy can be applied non-uniformly to create a gradient across the surface (e.g., high energy to low energy). The differential energy exposure can be achieved, for example, by source positioning relative to the sample or through the use of masks (either stationary or mobile). A variety of energy sources can be used including, but not limited to: UV, IR, visible light, corona discharge, plasma treatment, radio frequency glow discharge, and ion deposition with increasing current. Various combinations of chemistries and energy sources are known.

In an embodiment, an initiator is associated with the support, surface, or lawn. In an additional embodiment, an energy-activated reactant (e.g., UV initiator) is present in a concentration gradient across the solid support. Where the energy-activated reactant is present in a concentration gradient, the solid support is uniformly exposed to the energy source. In an additional embodiment, the energy-activated reactant is present in a concentration gradient and the energy is applied non-uniformly to create either a 1D or 2D gradient.

Methods of Using Gradients of Artificial Receptors or Building Blocks

The present gradient of artificial receptor or building block can be contacted with analyte or mixture of analytes by any of a variety of known methods. For example, the analyte or mixture of analytes can be flowed across the support. For example, the analyte or mixture of analytes can be placed in contact with an edge of the support and diffusion can draw the analyte or mixture into, onto, and/or across the support and/or gradient. During or after contact with the analyte or mixture of analytes, one or more LE wins can be flowed or diffused into, onto, and/or across the support and/or gradient.

The present gradient of artificial receptor or building block can be employed for separations of analytes such as proteins, nucleic acids, natural products, or the like in any of a variety of directions across a plate or slide. The present gradient of artificial receptor or building block can be employed for separating or characterizing mixtures of proteins, such as a proteome, e.g., a plasma proteome.

In an embodiment, the invention can include methods and/or gradients for binding or detecting a protein, one or more of a plurality of proteins, or a proteome. Methods and systems for detection can include methods and systems for clinical chemistry, environmental analysis, diagnostic assays, and for proteome analysis. For example, the gradient can be contacted with a sample including at least one protein or one proteome. The building blocks making up the gradient can be naïve to the test ligand. Then, binding of one or more proteins to the gradient can be detected. Next, the binding results can be interpreted to provide information about the sample, e.g., the proteome. In an embodiment, the invention includes a method for detecting a protein in a sample including contacting a gradient suitable for characterizing the protein with a sample suspected of containing the protein. The method can also include detecting or quantitating binding of the protein to the gradient.

FIG. 13 schematically illustrates an embodiment of a method for making and using a gradient for evaluating an analyte or mixture of analytes. The method can include probing a receptor array with a sample known to contain the analyte or mixture of analytes of interest. The analyte or mixture thereof can be a protein or mixture of proteins. Probing can be followed by selecting receptors suitable for binding an analyte of interest or distinguishing among a mixture of analytes of interest. The building blocks from the selected receptors can be employed for producing one or more gradients on a support. In an embodiment, a first gradient on the support can be a step gradient. In an embodiment, each step can be a receptor surface suitable for binding a particular analyte or analytes of interest. In an embodiment, a second gradient can be generally orthogonal to the first gradient. The second gradient can include variation on a characteristic of the building blocks such as described above with respect to FIGS. 4-9. The gradient on the support can then be probed to detect one or more of the analytes bound to the gradient.

In an embodiment, each analyte or mixture of analytes (e.g., protein or proteome) can provide a pattern of binding to the gradient. The pattern of binding can be characteristic of the analyte or mixture of analytes (e.g., protein or proteome) or a sample including the analyte or mixture of analytes (e.g., protein or proteome). The method can include storing a representation of the binding pattern as an image or a data structure. The representation of the binding pattern can be evaluated either by an operator or data processing system. The method can include such evaluating. A binding pattern from an unknown sample that matches the binding pattern for a particular protein then characterizes the unknown sample as containing that analyte or mixture of analytes (e.g., protein or proteome). A binding pattern from an unknown sample that matches the binding pattern for a particular proteome then characterizes the unknown sample as including or being that proteome or as including or being the organism or cell having that proteome. Similarly, a binding pattern from an unknown sample can be evaluated against the patterns of a plurality of particular analyte or mixture of analytes (e.g., protein or proteome) and the sample can be characterized as containing one or more of the analytes (e.g., protein or proteome). A plurality of binding patterns can be stored as a database.

An embodiment of the illustrated method can include creating a gradient. This embodiment can also include compiling a database of the binding patterns of specific analyte or mixture of analytes (e.g., protein or proteome), for example, by probing the gradient with a plurality of individual analytes (e.g., proteins or proteomes). Contacting the gradient with an unidentified analyte or mixture of analytes (e.g., protein or proteome) can create a test binding pattern. The method can then compare the test binding pattern with the binding patterns of known analytes or mixtures of analytes (e.g., proteins or proteomes) in the database in order to characterize or classify the unidentified analyte, protein, proteome, or cell or organism. In an embodiment, the database and the gradient have already been constructed and the method involves probing the gradient with an unknown analyte or mixture of analytes (e.g., protein or proteome) to create a test binding pattern and then comparing this binding pattern with the binding patterns in the database in order to characterize or classify the unidentified analyte, protein, proteome, or cell or organism.

A proteome gradient can be contacted with samples from an organism, cell, or tissue of interest. Proteins that bind to the proteome gradient can characterize or detect the organism, cell or tissue; can indicate a disorder caused by the organism or affecting the cell or tissue; can indicate successful therapy of a disorder caused by the organism or affecting the cell or tissue; characterize disease processes; identify therapeutic leads or strategies; or the like.

Molecular Descriptors

Any of a variety of molecular descriptors can be employed to describe or characterize the change that makes up a gradient. Suitable molecular descriptors include constitutional descriptors, electrostatic descriptors, geometrical descriptors, physicochemical descriptors, topological descriptors, and the like. Such descriptors are known and can be calculated using commercially available software packages.

Suitable constitutional descriptors include: formal charge, fraction of rotatable bonds, molecular formula, molecular weight, the number of aromatic bonds, the number of double bonds, the number of h-bond acceptors, the number of h-bond donors, the number of negative chargable groups, the number of negative-charged groups, the number of positive chargable groups, the number of positive-charged groups, the number of rigid bonds, the number of rings, the number of rotatable bonds, the number of single bonds, the number of total atoms, the number of triple bonds, ratio donors to acceptor, the number of negative-charged groups, the number of positive chargable groups, the number of positive-charged groups, functional groups. Such descriptors are known and can be calculated using commercially available software packages.

Suitable electrostatic descriptors include: charge polarization, local dipole index, maximum negative charge, maximum positive charge, maximum positive hydrogen charge, polarity parameter, relative negative charge, relative positive charge, total absolute atomic charge, total negative charge, total positive charge, charge partial surface area descriptors. Such descriptors are known and can be calculated using commercially available software packages.

Suitable charge partial surface area descriptors include: ACGD, charge on acceptors atoms 1st type (CHAA1), charge on acceptors atoms 2nd type (CHAA2) charge on acceptors atoms 3rd type (CHAA3), Charge on donatable hydrogens 1st type (CHDH1), Charge on donatable hydrogens 2nd type (CHDH2), Charge on donatable hydrogens 3rd type (CHDH3), CHGD, Difference in charged partial surface area (DPSA1), Difference in total charge weighted surface area (DPSA2), Difference in atomic charge weithed surface area (DPSA3), Fractional charged partial negative surface area 1st type (FNSA1), Fractional charged partial negative surface area 2nd type (FNSA2), Fractional charged partial negative surface area 3rd type (FNSA3), Fractional charged partial positive surface area 1st type (FPSA1), Fractional charged partial positive surface area 2nd type (FPSA2), Fractional charged partial positive surface area 3rd type (FPSA3), HRNCG, HRNCS, HRPCG, HRPCS, Hydrophobic SA-MPEOE, Negative charged polar SA ? MPEOE, Partial negative surface area 1st type (PNSA1), Partial negative surface area 2nd type (PNSA2), Partial negative surface area 3rd type (PNSA3), Positive charged polar SA-MPEOE, Partial positive surface area 1st type (PPSA1), Partial positive surface area 2nd type (PPSA2) Partial positive surface area 3rd type (PPSA3), Relative negative charge surface area (RNCS), Relative positive charge surface area (RPCS), Surface area on acceptor atoms 1st type (SAAA1), Surface area on acceptor atoms 2nd type (SAAA2), Surface area on acceptor atoms 3rd type (SAAA3), Surface area on donor hydrogens 1st type (SADH1), Surface area on donor hydrogens 2nd type (SADH2), Surface area on donor hydrogens 3rd type (SADH3), Surface weighted charged area on acceptor atoms 1st type (SCAA1), Surface weighted charged area on acceptor atoms 2nd type (SCAA2), Surface weighted charged area on acceptor atoms 3rd type (SCAA3), Surface weighted charged area on donor hydrogens 1st type (SCDH1), Surface weighted charged area on donor hydrogens 2nd type (SCDH2), Surface weighted charged area on donor hydrogens 3rd type (SCDH3), Surface weighted charged area on acceptor atoms 1st type (SCAA1), Surface weighted charged area on acceptor atoms 2nd type (SCAA2), Surface weighted charged area on acceptor atoms 3rd type (SCAA3), Surface weighted charged area on donor hydrogens 1st type (SCDH1), Surface weighted charged area on donor hydrogens 2nd type (SCDH2), Surface weighted charged area on donor hydrogens 3rd type (SCDH3), Surface weighted charged partial negative surface area 1st type (WNSA1), Surface weighted charged partial negative surface area 2nd type (WNSA2), Surface weighted charged partial negative surface area 3rd type (WNSA3), Surface weighted charged partial positive surface area 1st type (WPSA1), Surface weighted charged partial positive surface area 2nd type (WPSA2), Surface weighted charged partial positive surface area 3rd type (WPSA3). Such descriptors are known and can be calculated using commercially available software packages.

Suitable geometric descriptors include: 2D van der Waals surface area (VSA), 2D van der Waals volume, Fraction of 2D-VSA chargable groups, Fraction of 2D-VSA hydrophobic, Fraction of 2D-VSA polar, Topological Polar Surface Area, 2D van der Waals chemical features surface area. Such descriptors are known and can be calculated using commercially available software packages.

Suitable 2D van der Waals chemical features surface area descriptors include: 2D-VSA Hbond acceptor, 2D-VSA Hbond all, 2D-VSA Hbond donor, 2D-VSA hydrophobic, 2D-VSA hydrophobic_sat, 2D-VSA hydrophobic_unsat, 2D-VSA negative chargable groups, 2D-VSA other, 2D-VSA polar, 2D-VSA positive chargable groups. Such descriptors are known and can be calculated using commercially available software packages.

Suitable physicochemical descriptors include: AlogP98 value, AMR value, Buffer solubility, Polarizability_Miller, Polarizability_MPEOE, SK_BP, SK_MP, SKlogP value, SklogPvp, SKlogS value, SKlogS_buffer, Solvation Free Energy, Vapor pressure, Water solubility, AlogP98 atomic types. Such descriptors are known and can be calculated using commercially available software packages.

Suitable topological descriptors include: Autocorrelation descriptors (AlogP98) (e.g., ATS Geary 1˜10 AlogP98, ATS Moran 0˜10 AlogP98, ATS Moreau-Bruto 0˜10 AlogP98, ATS Moreau-Bruto 0˜10 AlogP98 average); Autocorrelation descriptors (Mass) (e.g., ATS Geary 1˜10 Mass, ATS Moran 0˜10 Mass, ATS Moreau-Bruto 0˜10 Mass, ATS Moreau-Bruto 0˜10 Mass average); Autocorrelation descriptors (Polarizability) (e.g., ATS Geary 1˜10 Polarizability, ATS Moran 0˜10 Polarizability, ATS Moreau-Bruto 0˜10 Polarizability, ATS Moreau-Bruto 0˜10 Polarizability average); Adjacency and distance matrix based descriptors (e.g., 1st Zagreb, 2-MTI, 2-MTI prime, 2nd Zagreb, Balaban index JX, Balaban index JY, Centralization_distance_matrix, Degree complexity, Dispersion_distance_matrix, Eccentric adjacency index, Eccentric connectivity index, Edge connectivity index, Edge Gutman MTI, Edge Hyper Wiener index, Edge MTI, Edge Wiener index, Graph diameter, Graph distance complexity, Graph distance index, Graph Petitjean, Graph radius, Graph vertex complexity, Gutman MTI, Harary index Hyper Wiener index, Mean distance deviation, Mean square distance index, Odd-even index, Platt number, Pogliani index, Quadratic index, Ramification index, Ring degree-distance index, Rouvray index, Superpendentic index, Unipolarity_distance_matrix, Variation_distance_matrix, Vertex degree-distance index, Wiener index, Xu); Atom-type Electrotopological state indices, E-state; Atom-type AI topological indices (AI); Autocorrelation descriptors (Charge) (e.g., ATS Geary 1˜10 Charge, ATS Moran 0˜10 Charge, ATS Moreau-Bruto 0˜10 Charge, ATS Moreau-Bruto 0˜10 Charge average); Autocorrelation descriptors (Electronegativity) (e.g., ATS Geary 1˜10 Electronegativity, ATS Moran 0˜10 Electronegativity, ATS Moreau-Bruto 0˜10 Electronegativity, ATS Moreau-Bruto 0˜10 Electronegativity average); Autocorrelation descriptors (E-state) (e.g., ATS Geary 1˜10 E-state ATS Moran 0˜10 E-state ATS Moreau-Bruto 0˜10 E-state ATS Moreau-Bruto 0˜10 E-state average); Autocorrelation descriptors (VDW radius) (e.g., ATS Geary 1˜10 VDW radius ATS Moran 0˜10 VDW radius ATS Moreau-Bruto 0˜10 VDW radius ATS Moreau-Bruto 0˜10 VDW radius average); BCUT descriptors (Charge) (e.g., BCUT highest eigenvalue 1˜5 MPEOE charge BCUT lowest eigenvalue 1˜5 MPEOE charge); BCUT descriptors (Electronegativity) (e.g., BCUT highest eigenvalue 1˜5 Electronegativity, BCUT lowest eigenvalue 1˜5 Electronegativity); BCUT descriptors (AlogP98) (e.g., BCUT highest eigenvalue 1˜5 AlogP98, BCUT lowest eigenvalue 1˜5 AlogP98); BCUT descriptors (Mass) (e.g., BCUT highest eigenvalue 1˜5 Mass BCUT lowest eigenvalue 1˜5 Mass); BCUT descriptors (Polarizability) (e.g., BCUT highest eigenvalue 1˜5 Polarizability, BCUT lowest eigenvalue 1˜5 Polarizability); BCUT descriptors (VDW radius) (e.g., BCUT highest eigenvalue 1˜5 VDW radius, BCUT lowest eigenvalue 1˜5 VDW radius); BCUT descriptors (E-state) (e.g., BCUT highest eigenvalue 1˜5 E-state, BCUT lowest eigenvalue 1˜5 E-state); Delta connectivity indices (e.g., Delta Chi 0, Delta Chi 1, Delta Chi 2, Delta Chi 3 cluster, Delta Chi 3 path, Delta Chi 4 cluster, Delta Chi 4 path, Delta Chi 4 path/cluster, Delta Chi 5 path); Difference connectivity indices (e.g., Difference chi 0, Difference chi 1, Difference chi 2, Difference chi 3, Difference chi 4, Difference chi 5); Galvez topological charge indices (e.g., Bound charge index 0˜10, Charge index 0˜10, Charge transfer index algebraic, Global topological charge index, Valence bound charge index 0˜10, Valence charge index 0˜10); Atom-type Hydrogen electrotopological state indices, HE-state; Information content related descriptors (e.g., BIC, CIC, I_adj_deg_equ, I_adj_deg_mag, I_adj_equ, I_adj_mag, I_dist_equ, I_dist_mag, I_edge_adj_deg_equ, I_edge_adj_deg_mag, I_edge_adj_equ, I_edge_adj_mag, I_edge_dist_equ, I_edge_dist_mag, IAC total, IC, SIC); Kappa indices (e.g., Kier alpha 1, Kier alpha 2, Kier alpha 3, Kier flexibility, Kier shape 1, Kier shape 2, Kier shape 3, Kier steric descriptor, Kier symmetry index); Kier & Hall molecular connectivity indices (e.g., Chi 0, Chi 1, Chi 2, Chi 3 cluster, Chi 3 path, Chi 4 cluster, Chi 4 path, Chi 4 path/cluster, Chi 5 path, Total structure connectivity index); Kier & Hall valence connectivity indices (e.g., VChi 0, VChi 1, VChi 2, VChi 3 cluster, VChi 3 path, VChi 4 cluster, VChi 4 path, VChi 4 path/cluster, VChi 5 path); Molecular walk count (e.g., Molecular walk count 2 Molecular walk count 3 Molecular walk count 4 Molecular walk count 5 Path/walk 2 Path/walk 3 Path/walk 4 Path/walk 5); Narumi topological indices (e.g., Narumi ATI, Narumi GTI, Narumi HTI); Subgraph count indices (e.g., SC-0, SC-1, SC-10 path, SC-2, SC-3 cluster, SC-3 path, SC-4 cluster, SC-4 path, SC-4 path/cluster, SC-5 path, SC-6 path, SC-7 path, SC-8 path, SC-9 path); Solvation molecular connectivity indices (e.g., Solvation chi 0, Solvation chi 1, Solvation chi 2, Solvation chi 3 cluster, Solvation chi 3 path, Solvation chi 4 cluster, Solvation chi 4 path, Solvation chi 4 path/cluster, Solvation chi 5 path); Valence shell count (e.g., VS-0, VS-1, VS-2, VS-3, VS-4, VS-5). Such descriptors are known and can be calculated using commercially available software packages.

Methods for Detecting

Contacting a gradient of an artificial receptor or building block with a test ligand can identify or characterize the test ligand. Binding of the test ligand to the gradient can produce a detectable signal. The detectable signal can be produced, for example, through mechanisms and properties such as scattering, absorbing or emitting light, producing or quenching fluorescence or luminescence, producing or quenching an electrical signal, and the like. Spectroscopic detection methods include use of labels or enzymes to produce light for detection by optical sensors or optical sensor gradients. The light can be ultraviolet, visible, or infrared light, which can be produced and/or detected through fluorescence, fluorescence polarization, chemiluminescence, bioluminescence, or chemibioluminescence.

Systems and methods for detecting electrical conduction, and changes in electrical conduction, include ellipsometry, surface plasmon resonance, capacitance, conductometry, surface acoustic wave, quartz crystal microbalance, Love-wave, infrared evanescent wave, enzyme labels with electrochemical detection, nanowire field effect transistors, MOSFETS—metal oxide semiconductor field effect transistors, CHEMFETS—organic membrane metal oxide semiconductor field effect transistors, ICP—intrinsically conducting polymers, FRET—fluorescence resonance energy transfer.

Apparatus that can detect binding to or signal from a gradient includes UV, visible, or infrared spectrometer, fluorescence or luminescence spectrometer, surface plasmon resonance, surface acoustic wave or quartz crystal microbalance detectors, pH, voltammetry or amperometry meters, radioisotope detector, or the like.

The detectable signal can originate from, for example, a signaling moiety incorporated into the gradient or a signaling moiety added to the gradient. The signal can also be intrinsic to the gradient or to the test ligand. The signal can come from, for example, the interaction of the test ligand with the gradient or the interaction of the test ligand with a signaling moiety that has been incorporated into the gradient. In an embodiment, the present method can include selecting a gradient for which binding induces a change in the signal from the signaling moiety, e.g., a fluorescent moiety. Such a change can signal binding to the gradient.

The present gradient can be part of products used in: analyzing a genome and/or proteome; pharmaceutical development; detectors for any of the test ligands; drug of abuse diagnostics or therapy; hazardous waste analysis or remediation; toxic agent alert or intervention; disease diagnostics or therapy; cancer diagnostics or therapy; food chain contamination analysis or remediation; and the like.

Test Ligands

The test ligand can be any ligand for which binding to an array or surface can be detected. The test ligand can be a pure compound, a mixture, or a “dirty” mixture containing a natural product or pollutant. Such dirty mixtures can be tissue homogenate, biological fluid, soil sample, water sample, or the like.

Test ligands include prostate specific antigen, other cancer markers, insulin, warfarin, other anti-coagulants, cocaine, other drugs-of-abuse, markers for E. coli, markers for Salmonella sp., markers for other food-borne toxins, food-borne toxins, markers for Smallpox virus, markers for anthrax, markers for other possible toxic biological agents, pharmaceuticals and medicines, pollutants and chemicals in hazardous waste, toxic chemical agents, markers of disease, pharmaceuticals, pollutants, biologically important cations (e.g., potassium or calcium ion), polynucleotides, peptides, carbohydrates, enzymes, bacteria, viruses, mixtures thereof, and the like. In certain embodiments, the test ligand can be at least one of small organic molecules, inorganic/organic complexes, metal ion, mixture of proteins, protein, nucleic acid, mixture of nucleic acids, mixtures thereof, and the like.

Suitable test ligands include any compound or category of compounds described elsewhere in this document as being a test ligand, including, for example, the microbes, proteins, cancer cells, drugs of abuse, and the like described above.

Methods of Making an Artificial Receptor

The present invention relates to a method of making an artificial receptor or immobilized combination of building blocks. In an embodiment, this method includes preparing a spot or region on a support, the spot or region including a plurality of building blocks immobilized on the support. The method can include forming a plurality of regions on a solid support, each region including a plurality of building blocks, and immobilizing (e.g., reversibly) a plurality of building blocks on the solid support in each region.

The method can include mixing a plurality of building blocks and employing the mixture in forming the region(s). Alternatively, the method can include immobilizing (e.g., reversibly) individual building blocks on the support. Coupling building blocks to the support can employ covalent bonding or noncovalent interactions. Suitable noncovalent interactions include interactions between ions, hydrogen bonding, van der Waals interactions, and the like. In an embodiment, the support can be functionalized with moieties that can engage in covalent bonding or noncovalent interactions. Forming regions can yield a gradient of heterogeneous combinations of building blocks. The method can apply or spot building blocks onto a support in combinations of 2, 3, 4, or more building blocks.

In an embodiment, the present method can be employed to produce a solid support having on its surface a plurality of regions, each region including a plurality of building blocks. For example, the method can include immobilizing on a glass slide in each of a plurality of regions a plurality of building blocks. Such a region can be referred to as including heterogeneous building blocks.

In an embodiment, the present method includes making a receptor surface. Making a receptor surface can include forming a region on a solid support, the region including a plurality of building blocks, and immobilizing (e.g., reversibly) the plurality of building blocks to the solid support in the region. The method can include mixing a plurality of building blocks and employing the mixture in forming the region or regions. Alternatively, the method can include applying individual building blocks in a region on the support. Forming a region on a support can be accomplished, for example, by soaking a portion of the support with the building block solution. The resulting coating including building blocks can be referred to as including heterogeneous building blocks.

A region including a plurality of building blocks can be independent and distinct from other regions including a plurality of building blocks. In an embodiment, one or more regions including a plurality of building blocks can overlap to produce a region including the combined pluralities of building blocks. In an embodiment, two or more regions including a single building block can overlap to form one or more regions each including a plurality of building blocks. The overlapping regions can be envisioned, for example, as portions of overlap in a Ven diagram, or as portions of overlap in a pattern like a plaid or tweed.

In an embodiment, the method produces a surface with a density of building blocks sufficient to provide interactions of more than one building block with a ligand. That is, the building blocks can be in proximity to one another. Proximity of different building blocks can be detected by determining different (e.g., greater) binding of a test ligand to a spot or surface including a plurality of building blocks compared to a spot or surface including only one of the building blocks.

In an embodiment, the method includes forming an array including one or more regions that function as controls for validating or evaluating binding to gradients of the present invention. In an embodiment, the method includes forming one or more regions that function as controls for validating or evaluating binding to gradients of the present invention. Such a control region can include no building block, only a single building block, only functionalized lawn, or combinations thereof.

The method can immobilize (e.g., reversibly) building blocks on supports using known methods for immobilizing compounds of the types employed as building blocks. Coupling building blocks to the support can employ covalent bonding or noncovalent interactions. Suitable noncovalent interactions include interactions between ions, hydrogen bonding, van der Waals interactions, and the like. In an embodiment, the support can be functionalized with moieties that can engage in reversible covalent bonding, moieties that can engage in noncovalent interactions, a mixture of these moieties, or the like.

In an embodiment, the support can be functionalized with moieties that can engage in covalent bonding, e.g., reversible covalent bonding. The present invention can employ any of a variety of the numerous known functional groups, reagents, and reactions for forming reversible covalent bonds. Suitable reagents for forming reversible covalent bonds include those described in Green, T W; Wuts, P G M (1999), Protective Groups in Organic Synthesis Third Edition, Wiley-Interscience, New York, 779 pp. For example, the support can include functional groups such as a carbonyl group, a carboxyl group, a silane group, boric acid or ester, an amine group (e.g., a primary, secondary, or tertiary amine, a hydroxylamine, a hydrazine, or the like), a thiol group, an alcohol group (e.g., primary, secondary, or tertiary alcohol), a diol group (e.g., a 1,2 diol or a 1,3 diol), a phenol group, a catechol group, or the like. These functional groups can form groups with reversible covalent bonds, such as ether (e.g., alkyl ether, silyl ether, thioether, or the like), ester (e.g., alkyl ester, phenol ester, cyclic ester, thioester, or the like), acetal (e.g., cyclic acetal), ketal (e.g., cyclic ketal), silyl derivative (e.g., silyl ether), boronate (e.g., cyclic boronate), amide, hydrazide, imine, carbamate, or the like. Such a functional group can be referred to as a covalent bonding moiety, e.g., a first covalent bonding moiety.

A carbonyl group on the support and an amine group on a building block can form an imine or Schiff's base. The same is true of an amine group on the support and a carbonyl group on a building block. A carbonyl group on the support and an alcohol group on a building block can form an acetal or ketal. The same is true of an alcohol group on the support and a carbonyl group on a building block. A thiol (e.g., a first thiol) on the support and a thiol (e.g., a second thiol) on the building block can form a disulfide.

A carboxyl group on the support and an alcohol group on a building block can form an ester. The same is true of an alcohol group on the support and a carboxyl group on a building block. Any of a variety of alcohols and carboxylic acids can form esters that provide covalent bonding that can be reversed in the context of the present invention. For example, reversible ester linkages can be formed from alcohols such as phenols with electron withdrawing groups on the aryl ring, other alcohols with electron withdrawing groups acting on the hydroxyl-bearing carbon, other alcohols, or the like; and/or carboxyl groups such as those with electron withdrawing groups acting on the acyl carbon (e.g., nitrobenzylic acid, R—CF₂—COOH, R—CCl₂—COOH, and the like), other carboxylic acids, or the like.

In an embodiment, the support, matrix, or lawn can be functionalized with moieties that can engage in noncovalent interactions. For example, the support can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like.

In an embodiment, the support, matrix, or lawn includes a charged moiety (e.g., a first charged moiety). Suitable charged moieties include positively charged moieties and negatively charged moieties. Suitable positively charged moieties (e.g., at neutral pH in aqueous compositions) include amines, quaternary ammonium moieties, ferrocene, or the like. Suitable negatively charged moieties (e.g., at neutral pH in aqueous compositions) include carboxylates, phenols substituted with strongly electron withdrawing groups (e.g., tetrachlorophenols), phosphates, phosphonates, phosphinates, sulphates, sulphonates, thiocarboxylates, hydroxamic acids, or the like.

In an embodiment, the support, matrix, or lawn includes groups that can hydrogen bond (e.g., a first hydrogen bonding group), either as donors or acceptors. The support, matrix, or lawn can include a surface or region with groups that can hydrogen bond. For example, the support, matrix, or lawn can include a surface or region including one or more carboxyl groups, amine groups, hydroxyl groups, carbonyl groups, or the like. Ionic groups can also participate in hydrogen bonding.

In an embodiment, the support, matrix, or lawn includes a lipophilic moiety (e.g., a first lipophilic moiety). Suitable lipophilic moieties include branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. A lipophilic moiety like a quaternary ammonium lipophilic moiety can also include a positive charge.

In an embodiment, lawn remaining after coupling of building blocks can be modified by acetylation, succinylation or like reactions (including modifications described hereinabove) to modify those groups that could but did not react with building block.

Artificial Receptors

A candidate artificial receptor, a lead artificial receptor, or a working artificial receptor includes combination of building blocks immobilized (e.g., reversibly) on, for example, a support. An individual artificial receptor can be a heterogeneous building block region on a slide or a plurality of building blocks coated on a slide, tube, or well. The building blocks can be immobilized through any of a variety of interactions, such as covalent, electrostatic, or hydrophobic interactions. For example, the building block and support or lawn can each include one or more functional groups or moieties that can form covalent, electrostatic, hydrogen bonding, van der Waals, or like interactions.

An gradient of candidate artificial receptors can be a commercial product sold to parties interested in using the gradients as implements in developing methods for detecting or characterizing test ligands of interest. In an embodiment, a useful gradient of candidate artificial receptors includes at least one glass slide, the at least one glass slide including a region including a gradient.

One or more lead artificial receptors can be developed from a plurality of candidate artificial receptors. In an embodiment, a lead artificial receptor includes a combination of building blocks and binds detectable quantities of test ligand upon exposure to, for example, several picomoles of test ligand at a concentration of 1, 0.1, or 0.01 μg/ml, or at 1, 0.1, or 0.01 ng/ml test ligand; at a concentration of 0.01 μg/ml, or at 1, 0.1, or 0.01 ng/ml test ligand; or a concentration of 1, 0.1, or 0.01 ng/ml test ligand.

In an embodiment, a gradient according to the present invention includes a combination of building blocks and binds categorizing or identifying quantities of test ligand upon exposure to, for example, several picomoles of test ligand at a concentration of 100, 10, 1, 0.1, 0.01, or 0.001 ng/ml test ligand; at a concentration of 10, 1, 0.1, 0.01, or 0.001 ng/ml test ligand; or a concentration of 1, 0.1, 0.01, or 0.001 ng/ml test ligand.

Artificial receptors, particularly candidate or lead artificial receptors, can be in the form of an array of artificial receptors. Each spot is a candidate artificial receptor and a combination of building blocks. The array can also be constructed to include lead artificial receptors. For example, the array of artificial receptors can include combinations of fewer building blocks and/or a subset of the building blocks. In an embodiment, an array of candidate artificial receptors includes building blocks of general Formula 2 (shown hereinbelow), with RE₁ being B1, B2, B3, B3a, B4, B5, B6, B7, B8, or B9 (shown hereinbelow) and with RE₂ being A1, A2, A3, A3a, A4, A5, A6, A7, A8, or A9 (shown hereinbelow). In an embodiment, the framework is tyrosine.

In an embodiment, the artificial receptor of the invention includes a plurality of building blocks coupled to a support. In an embodiment, the plurality of building blocks can include or be building blocks of Formula 2 (shown below). An abbreviation for the building block including a linker, a tyrosine framework, and recognition elements AxBy is TyrAxBy. In an embodiment, a candidate artificial receptor can include combinations of building blocks of formula TyrA1B1, TyrA2B2, TyrA2B4, TyrA2B6, TyrA2B8, TyrA3B3, TyrA4B2, TyrA4B4, TyrA4B6, TyrA4B8, TyrA5B5, TyrA6B2, TyrA6B4, TyrA6B6, TyrA6B8, TyrA7B7, TyrA8B2, TyrA8B4, TyrA8B6, or TyrA8B8.

The present artificial receptors can employ any of a variety of supports to which building blocks or other array materials can be coupled. For example, the support can be glass or plastic; a slide, a tube, or a well; an optical fiber; or the like.

The present invention includes obtaining a result employing a gradient according to the invention and communicating or forwarding that result. Further, the result obtained from the gradient can be processed are configured to provide patterns or conclusions. Communicating or forwarding such patterns or conclusions is also a part of the present invention. Forwarding or communicating can include forwarding or communicating between a first and second location, which can be remote from one another. Forwarding and communicating can include sending and/or receiving. In an embodiment, the present invention includes forwarding to a remote location a result obtained from a method employing a gradient, building block, or artificial receptor according to the present invention. In an embodiment, the present invention includes transmitting data representing a result or reading obtained employing a gradient, building block, or artificial receptor according to the present invention.

Remote objects can be separated by, for example, being in two different buildings, being one or more miles apart, being 10 or more miles apart, or being 100 or more miles apart. Communicating can include transmitting data or other information, for example, as electrical signals over any of a variety of transmission systems. Forwarding can include any mechanism for translocating an item such as data or other information from one location to another. Forwarding can include physically transporting or transmitting the item, data, or information.

Building Blocks

The present invention relates to building blocks for making or forming candidate artificial receptors. Building blocks can be designed, made, and selected to provide a variety of structural characteristics among a small number of compounds. A building block can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A building block can be bulky or it can be small.

A building block can be visualized as including several components, such as one or more frameworks, one or more linkers, and/or one or more recognition elements. The framework can be covalently coupled to each of the other building block components. The linker can be covalently coupled to the framework. The linker can be coupled to a support through one or more of covalent, electrostatic, hydrogen bonding, van der Waals, or like interactions. The recognition element can be covalently coupled to the framework. In an embodiment, a building block includes a framework, a linker, and a recognition element. In an embodiment, a building block includes a framework, a linker, and two recognition elements.

A description of general and specific features and functions of a variety of building blocks and their synthesis can be found in copending U.S. patent application Ser. No. 10/244,727, filed Sep. 16, 2002, Ser. No. 10/813,568, filed Mar. 29, 2004, and Application No. PCT/US03/05328, filed Feb. 19, 2003, each entitled “ARTIFICIAL RECEPTORS, BUILDING BLOCKS, AND METHODS”; U.S. patent application Ser. Nos. 10/812,850 and 10/813,612, and application No. PCT/US2004/009649, each filed Mar. 29, 2004 and each entitled “ARTIFICIAL RECEPTORS INCLUDING REVERSIBLY IMMOBILIZED BUILDING BLOCKS, THE BUILDING BLOCKS, AND METHODS”; and U.S. Provisional Patent Application Nos. 60/499,965, filed Sep. 3, 2003, and 60/526,699, filed Dec. 2, 2003, each entitled BUILDING BLOCKS FOR ARTIFICIAL RECEPTORS; the disclosures of which are incorporated herein by reference. These patent documents include, in particular, a detailed written description of: function, structure, and configuration of building blocks, framework moieties, recognition elements, synthesis of building blocks, specific embodiments of building blocks, specific embodiments of recognition elements, and sets of building blocks.

### Embodiments of Building Blocks

The building block can include one or more functional groups, structural features, or moieties that form the recognition moiety. For example, the building block can include one or more carboxyl, amine, hydroxyl, phenol, carbonyl, and thiol groups, which can be a recognition moiety. For example, the building block can include one or more moieties with positive charge, negative charge, acid, base, electron acceptor, electron donor, hydrogen bond donor, hydrogen bond acceptor, free electron pair, π electrons, charge polarization, hydrophilicity, hydrophobicity, and the like. The building block can include two, three, or four such functional groups, structural features, or moieties.

The building block can include one or more functional groups, structural features, or moieties that form all or part of the linking moiety. For example, the building block can include one or more carboxyl, amine, hydroxyl, phenol, carbonyl, and thiol groups, which can be a linking moiety. For example, the building block can include one or more moieties with positive charge, negative charge, acid, base, electron acceptor, electron donor, hydrogen bond donor, hydrogen bond acceptor, free electron pair, π electrons, charge polarization, hydrophilicity, hydrophobicity, and the like. The linking moiety is configured for coupling (e.g., reversibly) to the support.

A building block can be or can include any of a variety of compounds or substructures. For example, a building block can be or include an amino acid (natural or synthetic), a dipeptide, a monosaccharide, a disaccharide, another carbohydrate, a mixture or combination thereof, or the like; a catalytic moiety such as a coenzyme, a metal, a metal complex, or the like; a polymer of up to 2000 carbon atoms (e.g., up to 48 carbon atoms), e.g., a polyether, polyethyleneimine, a polyacrylamide, or like polymer; an α-hydroxy acid, a thioic acid; an enzyme inhibitor (e.g., a protease inhibitor (such as pepstatin), a statin, or the like), a receptor antagonist (e.g., a benzodiazepine), a receptor agonist, a pharmaceutical, a peptide hormone; a natural product, a starting material, intermediate, or end product of a metabolic pathway (e.g., glycolysis, the citric acid cycle, photosynthesis, glucogenesis, mitochondrial electron transport, oxidative phosphorylation, biosynthetic pathways, catabolic pathways, or the like); a mixture or combination thereof, or the like. A building block can be a naturally occurring or synthetic compound; can be racemic, optically active, or achiral; can include positional isomers of any specifically described structure; or can include conformationally restricted functional groups.

In an embodiment, the building block is or includes a monosaccharide. Any of a variety of naturally occurring or synthetic monosaccharides can be employed as a building block. Suitable monosaccharides include pyranoses and furanoses, such as glucose, fructose, ribulose, allose, altrose, mannose, gulose, idose, galactose, talose, ribose, arabinose, xylose, lyxose, or the like; erythrose, threose, or the like; inositol, or the like; amino sugars, such as rhammose, fucose, glucosamine, galactosamine, or the like; aldonic and uronic acids, such as gluconic acid, glucuronic acid, glucaric acid, or the like; glycosides including these monosaccharides; disaccharides or oligosaccharides including these monosaccharides, such as sucrose, raffinose, gentianose, cellobiose, maltose, lactose, trehalose, gentiobiose, meliobiose, or the like; a mixture or combination thereof, or the like.

In an embodiment, the building block is or includes a disaccharide. Any of a variety of naturally occurring or synthetic disaccharides can be employed as a building block. Suitable disaccharides include disaccharides or oligosaccharides including the monosaccharides listed above. Such disaccharides include sucrose, raffinose, gentianose, cellobiose, maltose, lactose, trehalose, gentiobiose, meliobiose, or the like; a mixture or combination thereof, or the like.

In an embodiment, the building block is or includes a carbohydrate. Any of a variety of naturally occurring or synthetic carbohydrates can be employed as a building block. Suitable carbohydrates include cellulose, chitin, starch, glycogen, hyaluronic acid, chondroitin sulfates, keratosulfate, heparin, glycoproteins, or the like; a mixture or combination thereof, or the like.

In an embodiment, the building block is or includes a catalytic moiety. Any of a variety of naturally occurring or synthetic catalytic moieties can be employed as or can be a moiety on a building block. Suitable catalytic moieties include coenzymes, metals, metal complexes, pronucleophiles, proelectrophiles, proreducing agents, prooxidizing agents, general acid catalysts, general base catalysts, a mixture or combination thereof, or the like.

In an embodiment, the building block is or includes a metal binding or complexing moiety. Any of a variety of naturally occurring or synthetic metal binding or complexing moieties can be employed as or can be a moiety on a building block. Suitable metal binding or complexing moieties include synthetic and naturally occurring porphyrin (e.g., etioporphyrin, mesoporphyrin, protoporphyrin (e.g., heme or hematin), coproporphyrin, tetraphenylporphyrin, octaethylporphyrin, or the like), a cobamide coenzyme (e.g., coenzyme B₁₂, a cobalamin such as methyl-cobalamin, or the like), selenocysteine, selenomethionine, ferritin; naturally occurring or synthetic complexes of magnesium, zinc, copper, chromium, iron, cobalt, aluminum (e.g., Al³⁺), titanium (e.g., Ti⁴⁺) or the like; salt thereof, a mixture or combination thereof, or the like.

In an embodiment, the building block is or includes a coenzyme (which can also be called a prosthetic group or cofactor). Any of a variety of naturally occurring or synthetic coenzymes can be employed as or can be a moiety on a building block. Suitable coenzymes include a nicotinamide coenzyme (e.g., NAD, NADH, NADP, NADPH, and the like), a flavin compound (e.g., FAD, FADH₂, FMN, FMNH₂), a lipoic acid (e.g., oxidized or reduced lipoic acid), a glutathione (e.g., oxidized or reduced glutathione), an ascorbic acid, a quinone (e.g., ubiquinone, vitamins K, or the like), a porphyrin (e.g., etioporphyrin, mesoporphyrin, protoporphyrin (e.g., heme or hematin), coproporphyrin, or the like), a nucleoside (e.g., adenine, guanine, cytosine, thymine, uracil), a nucleotide (e.g., AMP, ADP, ATP, GMP, GDP, GTP, CMP, CDP, CTP, TMP, TDP, TTP, UMP, UDP, UTP), a glycerol phosphate, a biotin (e.g., biotin or carboxybiotin), a pyridoxal (e.g., pyridoxal phosphate, pyridoxal, pyridoxamine, pyridoxamine phosphate, or Schiff's bases thereof), an oxoglutaric acid (e.g., 2-oxoglutarate), a coenzyme A, a carnitine, a folic acid (e.g., tetrahydrofolic acid, 5-formyltetrahydrofolic acid, 10-formyltetrahydrofolic acid, 5,10-methenyltetrahydrofolic acid, 5,10-methylenetetrahydrofolic acid, 5-hydroxymethyltetrahydrofolic acid, 5-formiminotetrahydrofolic acid, or the like), an adenosylhomocysteine, a cobamide coenzyme (e.g., coenzyme B₁₂, a cobalamin such as methyl-cobalamin, or the like), adenosine 3′,5′-bisphosphate, thiamin diphosphate, ferritin, salt thereof, a mixture or combination thereof, or the like.

In an embodiment, the building block is or includes a polymer of up to 2000 carbon atoms (e.g., up to 48 carbon atoms). Such a polymer can be naturally occurring or synthetic. Suitable polymers include a polyether or like polymer, such as a PEG, a polyethyleneimine, polyacrylate (e.g., substituted polyacrylates), salt thereof, a mixture or combination thereof, or the like. Suitable PEGs include PEG 1500 up to PEG 20,000, for example, PEG 1450, PEG 3350, PEG 4500, PEG 8000, PEG 20,000, and the like.

In an embodiment, the present building block can be or include a lipophilic moiety. Suitable lipophilic moieties include one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like.

Suitable building blocks include carboxylic acids (e.g., mono and di-carboxylates) with the carboxylate appended to a lipophilic moiety, such as one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); or the like. Such carboxylic acids include arachidonic acid, linoleic acid, linolenic acid, oleic acid, and the like. Such carboxylic acids can be immobilized on a support through covalent bonding or electrostatic interaction between.

Suitable building blocks include carboxylic acids (e.g., mono and di-carboxylates) with the carboxylate appended to a an organic radical, such as one or more branched or straight chain C₂₋₈ alkyl, arylalkyl, alkenyl, alkynyl, or the like. These carboxylic acids can include substituted aryl moieties (e.g., phenyl or naphthyl moieties). Such carboxylic acids include acetic acid, propionic acid, butanoic acid, pentanoic acid, hexanoic acid, heptanoic acid, octanoic acid, oxalic acid, malonic acid, succinic acid, glutaric acid, adipic acid, pimelic acid, benzoic acid, and the like. Such carboxylic acids can be immobilized on a support through covalent bonding or electrostatic interaction between the carboxyl(ate) and the support or lawn.

In an embodiment, the building block is or includes an amino acid. Suitable amino acids include a natural or synthetic amino acid. Amino acids include carboxyl and amine functional groups. In their side chains, amino acids can also include a moiety with one or more of positive charge, negative charge, acid, base, electron acceptor, electron donor, hydrogen bond donor, hydrogen bond acceptor, free electron pair, π electrons, charge polarization, hydrophilicity, or hydrophobicity. Suitable amino acids include those with a functional group on the side chain. The side chain functional group can include, for natural amino acids, an amine (e.g., alkyl amine, heteroaryl amine), hydroxyl, phenol, carboxyl, thiol, thioether, or amidino group.

Any of the natural amino acids can be employed as a building block. The natural amino acids include aliphatic amino acids (e.g., alanine, valine, leucine, and isoleucine), hydroxyamino acids (e.g., serine, threonine, and tyrosine), dicarboxylic acids (e.g., glutamic acid and aspartic acid), amides (e.g., glutamine and asparagine), amino acids with basic sidechains (e.g., lysine, hydroxylysine, histidine, and arginine), aromatic amino acids (e.g., histidine, phenylalanine, tyrosine, tryptophan, and thyroxine), sulfur containing amino acids (e.g., cysteine, cystine, and methionine), imino acids (e.g., proline and hydroxyproline). Natural amino acids suitable for use as building blocks include, for example, serine, threonine, tyrosine, aspartic acid, glutamic acid, asparagine, glutamine, cysteine, lysine, arginine, histidine.

Synthetic amino acids can include the naturally occurring side chain functional groups or synthetic side chain functional groups which modify or extend the natural amino acids with alkyl, substituted alkyl, cycloalkyl, heterocyclic, substituted heterocyclic, aryl alkyl, aryl, heteroaryl, heteroaryl alkyl, and like moieties and with carboxyl, amine, hydroxyl, phenol, carbonyl, or thiol functional groups. Suitable synthetic amino acids include N-substituted glycine and oligomers of N-substituted glycines. Suitable synthetic amino acids include β-amino acids and homo or β analogs of natural amino acids.

In an embodiment, the building block is or includes a dipeptide. Any of the 400 dipeptides including the 20 natural amino acids in any order can be employed as building blocks. Suitable dipeptides include muramyl dipeptide or the like.

In an embodiment the building block can be or include a therapeutic or pharmacologically active agent. Suitable therapeutic or pharmacologically active agents include a nitrate, nitric oxide, a nitric oxide promoter, nitric oxide donors, dipyridamole, or another vasodilator; HYTRIN® or another antihypertensive agent; a glycoprotein Ib/IIIa inhibitor (abciximab) or another inhibitor of surface glycoprotein receptors; aspirin, ticlopidine, clopidogrel or another antiplatelet agent; colchicine or another antimitotic, or another microtubule inhibitor; a retinoid or another antisecretory agent; cytochalasin or another actin inhibitor; methotrexate or another antimetabolite or antiproliferative agent; tamoxifen citrate, TAXOL®, paclitaxel, or derivatives thereof, rapamycin, vinblastine, vincristine, vinorelbine, etoposide, tenopiside, dactinomycin (actinomycin D), daunorubicin, doxorubicin, idarubicin, an anthracycline, mitoxantrone, bleomycin, plicamycin (mithramycin), mitomycin, mechlorethamine, cyclophosphamide and its analogs, chlorambucil, an ethylenimine, a methylmelamine, an alkyl sulfonate (e.g., busulfan), a nitrosourea (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, or other anti-cancer chemotherapeutic agents; cyclosporin, tacrolimus (FK-506), azathioprine, mycophenolate mofetil, mTOR inhibitors, or another immunosuppressive agent; cortisol, cortisone, dexamethasone, dexamethasone sodium phosphate, dexamethasone acetate, a dexamethasone derivative, betamethasone, fludrocortisone, prednisone, prednisolone, 6U-methylprednisolone, triamcinolone (e.g., triamcinolone acetonide), or another steroidal agent; trapidil (a PDGF antagonist); dopamine, bromocriptine mesylate, pergolide mesylate, or another dopamine agonist; captopril, enalapril or another angiotensin converting enzyme (ACE) inhibitor; angiotensin receptor blockers; ascorbic acid, alpha tocopherol, deferoxamine, a 21-aminosteroid (lasaroid) or another free radical scavenger, iron chelator or antioxidant; estrogen or another sex hormone; AZT or another antipolymerase; acyclovir, famciclovir, rimantadine hydrochloride, ganciclovir sodium, Norvir, Crixivan, α-methyl-1-adamantanemethylamine, hydroxy-ethoxymethylguanine, adamantanamine, 5-iodo-2′-deoxyuridine, trifluorothymidine, adenine arabinoside, or another antiviral agent; 5-aminolevulinic acid, meta-tetrahydroxyphenylchlorin, hexadecafluorozinc phthalocyanine, tetramethyl hematoporphyrin, rhodamine 123 or other photodynamic therapy agents; PROSCAR®, HYTRIN® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these.

In an embodiment, the building block can be or can include an antibiotic. Examples of antibiotics include penicillin, tetracycline, chloramphenicol, minocycline, doxycycline, vancomycin, bacitracin, kanamycin, neomycin, gentamycin, erythromycin and cephalosporins. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

In an embodiment, the building block can be or can include an enzyme inhibitor. Suitable enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatecho-1, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, allopurinol, and the like.

In an embodiment, the building block is or includes a signal element that produces a detectable signal when a test ligand is bound to the receptor. In an embodiment, the signal element can produce an optical signal or a electrochemical signal. Suitable optical signals include chemiluminescence or fluorescence. The signal element can be a fluorescent moiety. The fluorescent molecule can be one that is quenched by binding to the artificial receptor. For example, the signal element can be a molecule that fluoresces only when binding occurs. Suitable electrochemical signal elements include those that give rise to current or a potential. Suitable electrochemical signal elements include phenols and anilines, such as those with substitutents oriented ortho or para to one another, polynuclear aromatic hydrocarbons, sulfide-disulfide, sulfide-sulfoxide-sulfone, polyenes, polyeneynes, and the like. Suitable electrochemical signal elements include quinones and ferrocenes.

In an embodiment, the building block includes or is substituted with a moiety providing a positive charge (e.g., at neutral pH in aqueous compositions). Suitable positively charged moieties include one or more groups such as amines, quaternary ammonium moieties, sulfonium, phosphonium, ferrocene, and the like. Suitable amines include alkyl amines, alkyl diamines, heteroalkyl amines, aryl amines, heteroaryl amines, aryl alkyl amines, pyridines, heterocyclic amines (saturated or unsaturated, the nitrogen in the ring or not), amidines, hydrazines, and the like. Alkyl amines generally have 1 to 12 carbons, preferably 1-8, rings can have 3-12 carbons, preferably 3-8. Any of the amines can be employed as a quaternary ammonium compound. Additional suitable quaternary ammonium moieties include trimethyl alkyl quaternary ammonium moieties, dimethyl ethyl alkyl quaternary ammonium moieties, dimethyl alkyl quaternary ammonium moieties, aryl alkyl quaternary ammonium moieties, pyridinium quaternary ammonium moieties, and the like.

In an embodiment, the building block includes or is substituted with a moiety providing a negative charge (e.g., at neutral pH in aqueous compositions). Suitable negatively charged moieties include one or more groups such as carboxylates, phenols substituted with strongly electron withdrawing groups (e.g., substituted tetrachlorophenols), phosphates, phosphonates, phosphinates, sulphates, sulphonates, thiocarboxylates, and hydroxamic acids. Suitable carboxylates include alkyl carboxylates, aryl carboxylates, and aryl alkyl carboxylates. Suitable phosphates include phosphate mono-, di-, and tri-esters, and phosphate mono-, di-, and tri-amides. Suitable phosphonates include phosphonate mono- and di-esters, and phosphonate mono- and di-amides (e.g., phosphonamides). Suitable phosphinates include phosphinate esters and amides.

In an embodiment, the building block includes or is substituted with a moiety providing a negative charge and a positive charge (at neutral pH in aqueous compositions), such as sulfoxides, betaines, and amine oxides.

In an embodiment, the building block includes or is substituted with an acidic moiety. Suitable acidic moieties include one or more groups such as carboxylates, phosphates, sulphates, and phenols. Suitable acidic carboxylates include thiocarboxylates. Suitable acidic phosphates include the phosphates listed hereinabove.

In an embodiment, the building block includes or is substituted with a basic moiety. Suitable basic moieties include one or more groups such as amines. Suitable basic amines include alkyl amines, aryl amines, aryl alkyl amines, pyridines, heterocyclic amines (saturated or unsaturated, the nitrogen in the ring or not), amidines, and any additional amines listed hereinabove.

In an embodiment, the building block includes or is substituted with a hydrogen bond donor. Suitable hydrogen bond donors include one or more groups such as amines, amides, carboxyls, protonated phosphates, protonated phosphonates, protonated phosphinates, protonated sulphates, protonated sulphinates, alcohols, and thiols. Suitable amines include alkyl amines, aryl amines, aryl alkyl amines, pyridines, heterocyclic amines (saturated or unsaturated, the nitrogen in the ring or not), amidines, ureas, and any other amines listed hereinabove. Suitable protonated carboxylates, protonated phosphates include those listed hereinabove. Suitable alcohols include primary alcohols, secondary alcohols, tertiary alcohols, and aromatic alcohols (e.g., phenols).

In an embodiment, the building block includes or is substituted with a hydrogen bond acceptor or a moiety with one or more free electron pairs. Suitable groups can include one or more groups such as amines, amides, carboxylates, carboxyl groups, phosphates, phosphonates, phosphinates, sulphates, sulphonates, alcohols, ethers, thiols, and thioethers. Suitable amines include alkyl amines, aryl amines, aryl alkyl amines, pyridines, heterocyclic amines (saturated or unsaturated, the nitrogen in the ring or not), amidines, ureas, and amines as listed hereinabove. Suitable carboxylates include those listed hereinabove. Suitable phosphates, phosphonates and phosphinates include those listed hereinabove. Suitable alcohols include primary alcohols, secondary alcohols, tertiary alcohols, aromatic alcohols, and those listed hereinabove. Suitable ethers include alkyl ethers, aryl alkyl ethers.

In an embodiment, the building block includes or is substituted with a an uncharged polar or hydrophilic group. Suitable groups include one or more groups such as amides, alcohols, ethers, thiols, thioethers, esters, thio esters, boranes, borates, and metal complexes. Suitable alcohols include primary alcohols, secondary alcohols, tertiary alcohols, aromatic alcohols, and those listed hereinabove. Suitable ethers include those listed hereinabove.

In an embodiment, the building block includes or is substituted with an uncharged hydrophobic group. Suitable groups include one or more groups such as alkyl (substituted and unsubstituted), alkene (conjugated and unconjugated), alkyne (conjugated and unconjugated), aromatic. Suitable alkyl groups include lower alkyl, substituted alkyl, cycloalkyl, aryl alkyl, and heteroaryl alkyl. Suitable alkene groups include lower alkene and aryl alkene. Suitable aromatic groups include unsubstituted aryl, heteroaryl, substituted aryl, aryl alkyl, heteroaryl alkyl, alkyl substituted aryl, and polyaromatic hydrocarbons.

In an embodiment, the building block includes or is substituted with a spacer (e.g., small) moiety, such as hydrogen, methyl, ethyl, and the like.

Framework

The framework can be selected for functional groups that provide for coupling to the recognition moiety and for coupling to or being the linking moiety. The framework can interact with the ligand as part of the artificial receptor. In an embodiment, the framework includes multiple reaction sites with orthogonal and reliable functional groups and with controlled stereochemistry. Suitable functional groups with orthogonal and reliable chemistries include, for example, carboxyl, amine, hydroxyl, phenol, carbonyl, and thiol groups, which can be individually protected, deprotected, and derivatized. In an embodiment, the framework has two, three, or four functional groups with orthogonal and reliable chemistries. In an embodiment, the framework has three functional groups. In such an embodiment, the three functional groups can be independently selected, for example, from carboxyl, amine, hydroxyl, phenol, carbonyl, or thiol group. The framework can include alkyl, substituted alkyl, cycloalkyl, heterocyclic, substituted heterocyclic, aryl alkyl, aryl, heteroaryl, heteroaryl alkyl, and like moieties.

A general structure for a framework with three functional groups can be represented by Formula 1a: 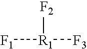
A general structure for a framework with four functional groups can be represented by Formula Ib: 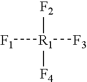
In these general structures: R₁ can be a 1-12, a 1-6, or a 1-4 carbon alkyl, substituted alkyl, cycloalkyl, heterocyclic, substituted heterocyclic, aryl alkyl, aryl, heteroaryl, heteroaryl alkyl, or like group; and F₁, F₂, F₃, or F₄ can independently be a carboxyl, amine, hydroxyl, phenol, carbonyl, or thiol group. F₁, F₂, F₃, or F₄ can independently be a 1-12, a 1-6, a 1-4 carbon alkyl, substituted alkyl, cycloalkyl, heterocyclic, substituted heterocyclic, aryl alkyl, aryl, heteroaryl, heteroaryl alkyl, or inorganic group substituted with carboxyl, amine, hydroxyl, phenol, carbonyl, or thiol group. F₃ and/or F₄ can be absent.

A variety of compounds fit the formulas and text describing the framework including amino acids, and naturally occurring or synthetic compounds including, for example, oxygen and sulfur functional groups. The compounds can be racemic, optically active, or achiral. For example, the compounds can be natural or synthetic amino acids, α-hydroxy acids, thioic acids, and the like.

Suitable molecules for use as a framework include a natural or synthetic amino acid, particularly an amino acid with a functional group (e.g., third functional group) on its side chain. Amino acids include carboxyl and amine functional groups. The side chain functional group can include, for natural amino acids, an amine (e.g., alkyl amine, heteroaryl amine), hydroxyl, phenol, carboxyl, thiol, thioether, or amidino group. Natural amino acids suitable for use as frameworks include, for example, serine, threonine, tyrosine, aspartic acid, glutamic acid, asparagine, glutamine, cysteine, lysine, arginine, histidine. Synthetic amino acids can include the naturally occurring side chain functional groups or synthetic side chain functional groups which modify or extend the natural amino acids with alkyl, substituted alkyl, cycloalkyl, heterocyclic, substituted heterocyclic, aryl alkyl, aryl, heteroaryl, heteroaryl alkyl, and like moieties as framework and with carboxyl, amine, hydroxyl, phenol, carbonyl, or thiol functional groups. Suitable synthetic amino acids include β-amino acids and homo or β analogs of natural amino acids. In an embodiment, the framework amino acid can be serine, threonine, or tyrosine, e.g., serine or tyrosine, e.g., tyrosine.

Although not limiting to the present invention, a framework amino acid, such as serine, threonine, or tyrosine, with a linker and two recognition elements can be visualized with one of the recognition elements in a pendant orientation and the other in an equatorial orientation, relative to the extended carbon chain of the framework.

All of the naturally occurring and many synthetic amino acids are commercially available. Further, forms of these amino acids derivatized or protected to be suitable for reactions for coupling to recognition element(s) and/or linkers can be purchased or made by known methods (see, e.g., Green, T W; Wuts, P G M (1999), Protective Groups in Organic Synthesis Third Edition, Wiley-Interscience, New York, 779 pp.; Bodanszky, M.; Bodanszky, A. (1994), The Practice of Peptide Synthesis Second Edition, Springer-Verlag, New York, 217 pp.).

### Embodiments of Frameworks

A framework can be or can include any of a variety of compounds or substructures. For example, a framework can be or include an amino acid (natural or synthetic), a dipeptide, a monosaccharide, a disaccharide, another carbohydrate, a mixture or combination thereof, or the like; a catalytic moiety such as a coenzyme, a metal, a metal complex, or the like; a polymer of up to 2000 carbon atoms (e.g., up to 48 carbon atoms), e.g., a polyether, polyethyleneimine, a polyacrylamide, or like polymer; an α-hydroxy acid, a thioic acid; an enzyme inhibitor (e.g., a protease inhibitor (such as pepstatin), a statin, or the like), a receptor antagonist (e.g., a benzodiazepine), a receptor agonist, a pharmaceutical, a peptide hormone; a natural product, a starting material, intermediate, or end product of a metabolic pathway (e.g., glycolysis, the citric acid cycle, photosynthesis, glucogenesis, mitochondrial electron transport, oxidative phosphorylation, biosynthetic pathways, catabolic pathways, or the like); a mixture or combination thereof, or the like. A framework can be a naturally occurring or synthetic compound; can be racemic, optically active, or achiral; can include positional isomers of any specifically described structure; or can include conformationally restricted functional groups.

In an embodiment, the framework is or includes a monosaccharide. Any of a variety of naturally occurring or synthetic monosaccharides can be employed as a framework. Suitable monosaccharides include pyranoses and furanoses, such as glucose, fructose, ribulose, allose, altrose, mannose, gulose, idose, galactose, talose, ribose, arabinose, xylose, lyxose, or the like; erythrose, threose, or the like; inositol, or the like; amino sugars, such as rhammose, fucose, glucosamine, galactosamine, or the like; aldonic and uronic acids, such as gluconic acid, glucuronic acid, glucaric acid, or the like; glycosides including these monosaccharides; a mixture or combination thereof, or the like.

In an embodiment, the framework is or includes a disaccharide. Any of a variety of naturally occurring or synthetic disaccharides can be employed as a framework. Suitable disaccharides include disaccharides or oligosaccharides including the monosaccharides listed above. Such disaccharides include sucrose, raffinose, gentianose, cellobiose, maltose, lactose, trehalose, gentiobiose, meliobiose, a mixture or combination thereof, or the like.

In an embodiment, the framework is or includes a carbohydrate. Any of a variety of naturally occurring or synthetic carbohydrates can be employed as a framework. Suitable carbohydrates include cellulose, chitin, starch, glycogen, hyaluronic acid, chondroitin sulfates, keratosulfate, heparin, glycoproteins, or the like; a mixture or combination thereof, or the like.

In an embodiment, the framework is or includes a catalytic moiety. Any of a variety of naturally occurring or synthetic catalytic moieties can be employed as or can be a moiety on a framework. Suitable catalytic moieties include coenzymes, metals, metal complexes, pronucleophiles, proelectrophiles, proreducing agents, prooxidizing agents, general acid catalysts, general base catalysts, a mixture or combination thereof, or the like.

In an embodiment, the framework is or includes a metal binding or complexing moiety. Any of a variety of naturally occurring or synthetic metal binding or complexing moieties can be employed as or can be a moiety on a framework. Suitable metal binding or complexing moieties include synthetic and naturally occurring porphyrin (e.g., etioporphyrin, mesoporphyrin, protoporphyrin (e.g., heme or hematin), coproporphyrin, tetraphenylporphyrin, octaethylporphyrin, or the like), a cobamide coenzyme (e.g., coenzyme B₁₂, a cobalamin such as methyl-cobalamin, or the like), selenocysteine, selenomethionine, ferritin; naturally occurring or synthetic complexes of magnesium, zinc, copper, chromium, iron, cobalt, aluminum (e.g., Al³⁺), titanium (e.g., Ti⁴⁺) or the like; salt thereof, a mixture or combination thereof, or the like.

In an embodiment, the framework is or includes a coenzyme (which can also be called a prosthetic group or cofactor). Any of a variety of naturally occurring or synthetic coenzymes can be employed as or can be a moiety on a framework. Suitable coenzymes include a nicotinamide coenzyme (e.g., NAD, NADH, NADP, NADPH, and the like), a flavin compound (e.g., FAD, FADH₂, FMN, FMNH₂), a lipoic acid (e.g., oxidized or reduced lipoic acid), a glutathione (e.g., oxidized or reduced glutathione), an ascorbic acid, a quinone (e.g., ubiquinone, vitamins K, or the like), a porphyrin (e.g., etioporphyrin, mesoporphyrin, protoporphyrin (e.g., heme or hematin), coproporphyrin, or the like), a nucleoside (e.g., adenine, guanine, cytosine, thymine, uracil), a nucleotide (e.g., AMP, ADP, ATP, GMP, GDP, GTP, CMP, CDP, CTP, TMP, TDP, TTP, UMP, UDP, UTP), a glycerol phosphate, a biotin (e.g., biotin or carboxybiotin), a pyridoxal (e.g., pyridoxal phosphate, pyridoxal, pyridoxamine, pyridoxamine phosphate, or Schiff's bases thereof), an oxoglutaric acid (e.g., 2-oxoglutarate), a coenzyme A, a carnitine, a folic acid (e.g., tetrahydrofolic acid, 5-formyltetrahydrofolic acid, 10-formyltetrahydrofolic acid, 5,10-methenyltetrahydrofolic acid, 5,10-methylenetetrahydrofolic acid, 5-hydroxymethyltetrahydrofolic acid, 5-formiminotetrahydrofolic acid, or the like), an adenosylhomocysteine, a cobamide coenzyme (e.g., coenzyme B₁₂, a cobalamin such as methyl-cobalamin, or the like), adenosine 3′,5′-bisphosphate, thiamin diphosphate, ferritin, salt thereof, a mixture or combination thereof, or the like.

In an embodiment, the framework is or includes a polymer of up to 2000 carbon atoms (e.g., up to 48 carbon atoms). Such a polymer can be naturally occurring or synthetic. Such a polymer can be naturally occurring or synthetic. Suitable polymers include a polyether or like polymer, such as a PEG, a polyethyleneimine, polyacrylate (e.g., substituted polyacrylates), salt thereof, a mixture or combination thereof, or the like. Suitable PEGs include PEG 1500 up to PEG 20,000, for example, PEG 1450, PEG 3350, PEG 4500, PEG 8000, PEG 20,000, and the like.

In an embodiment, the building block is or includes a dipeptide. Any of the 400 dipeptides including the 20 natural amino acids in any order can be employed as building blocks. Suitable dipeptides include muramyl dipeptide or the like.

In an embodiment the framework can be or include a therapeutic or pharmacologically active agent. Suitable therapeutic or pharmacologically active agents include a nitrate, nitric oxide, a nitric oxide promoter, nitric oxide donors, dipyridamole, or another vasodilator; HYTRIN® or another antihypertensive agent; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; a retinoid or another antisecretory agent; cytochalasin or another actin inhibitor; methotrexate or another antimetabolite or antiproliferative agent; tamoxifen citrate, TAXOL®, paclitaxel, or derivatives thereof, rapamycin, vinblastine, vincristine, vinorelbine, etoposide, tenopiside, dactinomycin (actinomycin D), daunorubicin, doxorubicin, idarubicin, an anthracycline, mitoxantrone, bleomycin, plicamycin (mithramycin), mitomycin, mechlorethamine, cyclophosphamide and its analogs, chlorambucil, an ethylenimine, a methylmelamine, an alkyl sulfonate (e.g., busulfan), a nitrosourea (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, or other anti-cancer chemotherapeutic agents; cyclosporin, tacrolimus (FK-506), azathioprine, mycophenolate mofetil, mTOR inhibitors, or another immunosuppressive agent; cortisol, cortisone, dexamethasone, dexamethasone sodium phosphate, dexamethasone acetate, a dexamethasone derivative, betamethasone, fludrocortisone, prednisone, prednisolone, 6U-methylprednisolone, triamcinolone (e.g., triamcinolone acetonide), or another steroidal agent; trapidil (a PDGF antagonist); dopamine, bromocriptine mesylate, pergolide mesylate, or another dopamine agonist; captopril, enalapril or another angiotensin converting enzyme (ACE) inhibitor; angiotensin receptor blockers; ascorbic acid, alpha tocopherol, deferoxamine, a 21-aminosteroid (lasaroid) or another free radical scavenger, iron chelator or antioxidant; estrogen or another sex hormone; AZT or another antipolymerase; acyclovir, famciclovir, rimantadine hydrochloride, ganciclovir sodium, Norvir, Crixivan, α-methyl-1-adamantanemethylamine, hydroxy-ethoxymethylguanine, adamantanamine, 5-iodo-2′-deoxyuridine, trifluorothymidine, adenine arabinoside, or another antiviral agent; 5-aminolevulinic acid, meta-tetrahydroxyphenylchlorin, hexadecafluorozinc phthalocyanine, tetramethyl hematoporphyrin, rhodamine 123 or other photodynamic therapy agents; PROSCAR®, HYTRIN® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these.

In an embodiment, the framework can be or can include an antibiotic. Examples of antibiotics include penicillin, tetracycline, chloramphenicol, minocycline, doxycycline, vancomycin, bacitracin, kanamycin, neomycin, gentamycin, erythromycin and cephalosporins. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

In an embodiment, the framework can be or can include an enzyme inhibitor. Suitable enzyme inhibitors include edrophonium chloride, N-methylphysostigmine, neostigmine bromide, physostigrnine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatecho-1, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, allopurinol, and the like.

In an embodiment, the framework is or includes a signal element that produces a detectable signal when a test ligand is bound to the receptor. In an embodiment, the signal element can produce an optical signal or a electrochemical signal. Suitable optical signals include chemiluminescence or fluorescence. The signal element can be a fluorescent moiety. The fluorescent molecule can be one that is quenched by binding to the artificial receptor. For example, the signal element can be a molecule that fluoresces only when binding occurs. Suitable electrochemical signal elements include those that give rise to current or a potential. Suitable electrochemical signal elements include phenols and anilines, such as those with substitutents oriented ortho or para to one another, polynuclear aromatic hydrocarbons, sulfide-disulfide, sulfide-sulfoxide-sulfone, polyenes, polyeneynes, and the like. Suitable electrochemical signal elements include quinones and ferrocenes.

Recognition Element

The recognition element can be selected to provide one or more structural characteristics to the building block. The recognition element can interact with the ligand as part of the artificial receptor. For example, the recognition element can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A recognition element can be a small group or it can be bulky.

In an embodiment the recognition element can be a 1-12, a 1-6, or a 1-4 carbon alkyl, substituted alkyl, cycloalkyl, heterocyclic, substituted heterocyclic, aryl alkyl, aryl, heteroaryl, heteroaryl alkyl, or like group. The recognition element can be substituted with a group that includes or imparts positive charge, negative charge, acid, base, electron acceptor, electron donor, hydrogen bond donor, hydrogen bond acceptor, free electron pair, π electrons, charge polarization, hydrophilicity, hydrophobicity, and the like.

### Embodiments of Recognition Elements

Recognition elements with a positive charge (e.g., at neutral pH in aqueous compositions) include amines, quaternary ammonium moieties, sulfonium, phosphonium, ferrocene, and the like. Suitable amines include alkyl amines, alkyl diamines, heteroalkyl amines, aryl amines, heteroaryl amines, aryl alkyl amines, pyridines, heterocyclic amines (saturated or unsaturated, the nitrogen in the ring or not), amidines, hydrazines, and the like. Alkyl amines generally have 1 to 12 carbons, e.g., 1-8, and rings can have 3-12 carbons, e.g., 3-8. Suitable alkyl amines include that of formula B9. Suitable heterocyclic or alkyl heterocyclic amines include that of formula A9. Suitable pyridines include those of formulas A5 and B5. Any of the amines can be employed as a quaternary ammonium compound. Additional suitable quaternary ammonium moieties include trimethyl alkyl quaternary ammonium moieties, dimethyl ethyl alkyl quaternary ammonium moieties, dimethyl alkyl quaternary ammonium moieties, aryl alkyl quaternary ammonium moieties, pyridinium quaternary ammonium moieties, and the like.

Recognition elements with a negative charge (e.g., at neutral pH in aqueous compositions) include carboxylates, phenols substituted with strongly electron withdrawing groups (e.g., substituted tetrachlorophenols), phosphates, phosphonates, phosphinates, sulphates, sulphonates, thiocarboxylates, and hydroxamic acids. Suitable carboxylates include alkyl carboxylates, aryl carboxylates, and aryl alkyl carboxylates. Suitable phosphates include phosphate mono-, di-, and tri-esters, and phosphate mono-, di-, and tri-amides. Suitable phosphonates include phosphonate mono- and di-esters, and phosphonate mono- and di-amides (e.g., phosphonamides). Suitable phosphinates include phosphinate esters and amides.

Recognition elements with a negative charge and a positive charge (at neutral pH in aqueous compositions) include sulfoxides, betaines, and amine oxides.

Acidic recognition elements can include carboxylates, phosphates, sulphates, and phenols. Suitable acidic carboxylates include thiocarboxylates. Suitable acidic phosphates include the phosphates listed hereinabove.

Basic recognition elements include amines. Suitable basic amines include alkyl amines, aryl amines, aryl alkyl amines, pyridines, heterocyclic amines (saturated or unsaturated, the nitrogen in the ring or not), amidines, and any additional amines listed hereinabove. Suitable alkyl amines include that of formula B9. Suitable heterocyclic or alkyl heterocyclic amines include that of formula A9. Suitable pyridines include those of formulas A5 and B5.

Recognition elements including a hydrogen bond donor include amines, amides, carboxyls, protonated phosphates, protonated phosphonates, protonated phosphinates, protonated sulphates, protonated sulphinates, alcohols, and thiols. Suitable amines include alkyl amines, aryl amines, aryl alkyl amines, pyridines, heterocyclic amines (saturated or unsaturated, the nitrogen in the ring or not), amidines, ureas, and any other amines listed hereinabove. Suitable alkyl amines include that of formula B9. Suitable heterocyclic or alkyl heterocyclic amines include that of formula A9. Suitable pyridines include those of formulas A5 and B5. Suitable protonated carboxylates, protonated phosphates include those listed hereinabove. Suitable amides include those of formulas A8 and B8. Suitable alcohols include primary alcohols, secondary alcohols, tertiary alcohols, and aromatic alcohols (e.g., phenols). Suitable alcohols include those of formulas A7 (a primary alcohol) and B7 (a secondary alcohol).

Recognition elements including a hydrogen bond acceptor or one or more free electron pairs include amines, amides, carboxylates, carboxyl groups, phosphates, phosphonates, phosphinates, sulphates, sulphonates, alcohols, ethers, thiols, and thioethers. Suitable amines include alkyl amines, aryl amines, aryl alkyl amines, pyridines, heterocyclic amines (saturated or unsaturated, the nitrogen in the ring or not), amidines, ureas, and amines as listed hereinabove. Suitable alkyl amines include that of formula B9. Suitable heterocyclic or alkyl heterocyclic amines include that of formula A9. Suitable pyridines include those of formulas A5 and B5. Suitable carboxylates include those listed hereinabove. Suitable amides include those of formulas A8 and B8. Suitable phosphates, phosphonates and phosphinates include those listed hereinabove. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include alkyl ethers, aryl alkyl ethers. Suitable alkyl ethers include that of formula A6. Suitable aryl alkyl ethers include that of formula A4. Suitable thioethers include that of formula B6.

Recognition elements including uncharged polar or hydrophilic groups include amides, alcohols, ethers, thiols, thioethers, esters, thio esters, boranes, borates, and metal complexes. Suitable amides include those of formulas A8 and B8. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include those listed hereinabove. Suitable ethers include that of formula A6. Suitable aryl alkyl ethers include that of formula A4.

Recognition elements including uncharged hydrophobic groups include alkyl (substituted and unsubstituted), alkene (conjugated and unconjugated), alkyne (conjugated and unconjugated), aromatic. Suitable alkyl groups include lower alkyl, substituted alkyl, cycloalkyl, aryl alkyl, and heteroaryl alkyl. Suitable lower alkyl groups include those of formulas A1, A3, A3a, and B1. Suitable aryl alkyl groups include those of formulas A3, A3a, A4, B3, B3a, and B4. Suitable alkyl cycloalkyl groups include that of formula B2. Suitable alkene groups include lower alkene and aryl alkene. Suitable aryl alkene groups include that of formula B4. Suitable aromatic groups include unsubstituted aryl, heteroaryl, substituted aryl, aryl alkyl, heteroaryl alkyl, alkyl substituted aryl, and polyaromatic hydrocarbons. Suitable aryl alkyl groups include those of formulas A3, A3a and B4. Suitable alkyl heteroaryl groups include those of formulas A5 and B5.

Spacer (e.g., small) recognition elements include hydrogen, methyl, ethyl, and the like. Bulky recognition elements include 7 or more carbon or hetero atoms.

Formulas A1-A9 and B1-B9 are:

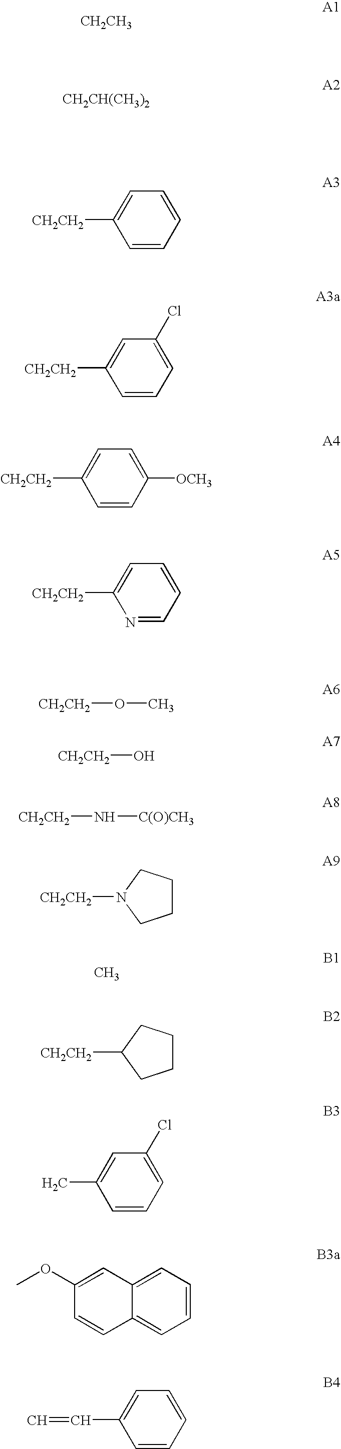

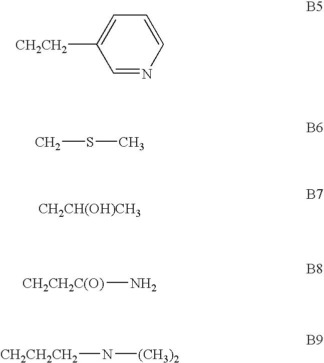

These A and B recognition elements can be called derivatives of, according to a standard reference: A1, ethylamine; A2, isobutylamine; A3, phenethylamine; A4, 4-methoxyphenethylamine; A5, 2-(2-aminoethyl)pyridine; A6, 2-methoxyethylamine; A7, ethanolamine; A8, N-acetylethylenediamine; A9, 1-(2-aminoethyl)pyrrolidine; B1, acetic acid, B2, cyclopentylpropionic acid; B3, 3-chlorophenylacetic acid; B4, cinnamic acid; B5, 3-pyridinepropionic acid; B6, (methylthio)acetic acid; B7, 3-hydroxybutyric acid; B8, succinamic acid; and B9, 4-(dimethylamino)butyric acid.

In an embodiment, the recognition elements include one or more of the structures represented by formulas A1, A2, A3, A3a, A4, A5, A6, A7, A8, and/or A9 (the A recognition elements) and/or B1, B2, B3, B3a, B4, B5, B6, B7, B8, and/or B9 (the B recognition elements). In an embodiment, each building block includes an A recognition element and a B recognition element. In an embodiment, a group of 81 such building blocks includes each of the 81 unique combinations of an A recognition element and a B recognition element. In an embodiment, the A recognition elements are linked to a framework at a pendant position. In an embodiment, the B recognition elements are linked to a framework at an equatorial position. In an embodiment, the A recognition elements are linked to a framework at a pendant position and the B recognition elements are linked to the framework at an equatorial position.

Although not limiting to the present invention, it is believed that the A and B recognition elements represent the assortment of functional groups and geometric configurations employed by polypeptide receptors. Although not limiting to the present invention, it is believed that the A recognition elements represent six advantageous functional groups or configurations and that the addition of functional groups to several of the aryl groups increases the range of possible binding interactions. Although not limiting to the present invention, it is believed that the B recognition elements represent six advantageous functional groups, but in different configurations than employed for the A recognition elements. Although not limiting to the present invention, it is further believed that this increases the range of binding interactions and further extends the range of functional groups and configurations that is explored by molecular configurations of the building blocks.

In an embodiment, the building blocks including the A and B recognition elements can be visualized as occupying a binding space defined by lipophilicity/hydrophilicity and volume. A volume can be calculated (using known methods) for each building block including the various A and B recognition elements. A measure of lipophilicity/hydrophilicity (logP) can be calculated (using known methods) for each building block including the various A and B recognition elements. Negative values of logP show affinity for water over nonpolar organic solvent and indicate a hydrophilic nature. A plot of volume versus logP can then show the distribution of the building blocks through a binding space defined by size and lipophilicity/hydrophilicity.

Reagents that form many of the recognition elements are commercially available. For example, reagents for forming recognition elements A1, A2, A3, A3a, A4, A5, A6, A7, A8, A9 B1, B2, B3, B3a, B4, B5, B6, B7, B8, and B9 are commercially available.

### Additional Embodiments of Recognition Elements

A recognition element can be or can include any of a variety of compounds or substructures. For example, a recognition element can be or include an amino acid (natural or synthetic), a dipeptide, a monosaccharide, a disaccharide, another carbohydrate, a mixture or combination thereof, or the like; a catalytic moiety such as a coenzyme, a metal, a metal complex, or the like; a polymer of up to 2000 carbon atoms (e.g., up to 48 carbon atoms), e.g., a polyether, polyethyleneimine, a polyacrylamide, or like polymer; an α-hydroxy acid, a thioic acid; an enzyme inhibitor (e.g., a protease inhibitor (such as pepstatin), a statin, or the like), a receptor antagonist (e.g., a benzodiazepine), a receptor agonist, a pharmaceutical, a peptide hormone; a natural product, a starting material, intermediate, or end product of a metabolic pathway (e.g., glycolysis, the citric acid cycle, photosynthesis, glucogenesis, mitochondrial electron transport, oxidative phosphorylation, biosynthetic pathways, catabolic pathways, or the like); a mixture or combination thereof, or the like. A building block can be a naturally occurring or synthetic compound; can be racemic, optically active, or achiral; can include positional isomers of any specifically described structure; or can include conformationally restricted functional groups.

In an embodiment, the recognition element is or includes a monosaccharide. Any of a variety of naturally occurring or synthetic monosaccharides can be employed as a recognition element. Suitable monosaccharides include pyranoses and furanoses, such as glucose, fructose, ribulose, allose, altrose, mannose, gulose, idose, galactose, talose, ribose, arabinose, xylose, lyxose, or the like; erythrose, threose, or the like; inositol, or the like; amino sugars, such as rhammose, fucose, glucosamine, galactosamine, or the like; aldonic and uronic acids, such as gluconic acid, glucuronic acid, glucaric acid, or the like; glycosides including these monosaccharides; a mixture or combination thereof, or the like.

In an embodiment, the recognition element is or includes a disaccharide. Any of a variety of naturally occurring or synthetic disaccharides can be employed as a building block. Suitable disaccharides include disaccharides or oligosaccharides including the monosaccharides listed above. Such disaccharides include sucrose, raffinose, gentianose, cellobiose, maltose, lactose, trehalose, gentiobiose, meliobiose, a mixture or combination thereof, or the like.

In an embodiment, the recognition element is or includes a carbohydrate. Any of a variety of naturally occurring or synthetic carbohydrates can be employed as a recognition element. Suitable carbohydrates include cellulose, chitin, starch, glycogen, hyaluronic acid, chondroitin sulfates, keratosulfate, heparin, glycoproteins, or the like; a mixture or combination thereof, or the like.

In an embodiment, the recognition element is or includes a catalytic moiety. Any of a variety of naturally occurring or synthetic catalytic moieties can be employed as or can be a moiety on a recognition element. Suitable catalytic moieties include coenzymes, metals, metal complexes, pronucleophiles, proelectrophiles, proreducing agents, prooxidizing agents, general acid catalysts, general base catalysts, a mixture or combination thereof, or the like.

In an embodiment, the recognition element is or includes a metal binding or complexing moiety. Any of a variety of naturally occurring or synthetic metal binding or complexing moieties can be employed as or can be a moiety on a recognition element. Suitable metal binding or complexing moieties include synthetic and naturally occurring porphyrin (e.g., etioporphyrin, mesoporphyrin, protoporphyrin (e.g., heme or hematin), coproporphyrin, tetraphenylporphyrin, octaethylporphyrin, or the like), a cobamide coenzyme (e.g., coenzyme B₁₂, a cobalamin such as methyl-cobalamin, or the like), selenocysteine, selenomethionine, ferritin; naturally occurring or synthetic complexes of magnesium, zinc, copper, chromium, iron, cobalt, aluminum (e.g., Al³⁺), titanium (e.g., Ti⁴⁺) or the like; salt thereof, a mixture or combination thereof, or the like.

In an embodiment, the recognition element is or includes a coenzyme (which can also be called a prosthetic group or cofactor). Any of a variety of naturally occurring or synthetic coenzymes can be employed as or can be a moiety on a recognition element. Suitable coenzymes include a nicotinamide coenzyme (e.g., NAD, NADH, NADP, NADPH, and the like), a flavin compound (e.g., FAD, FADH₂, FMN, FMNH₂), a lipoic acid (e.g., oxidized or reduced lipoic acid), a glutathione (e.g., oxidized or reduced glutathione), an ascorbic acid, a quinone (e.g., ubiquinone, vitamins K, or the like), a porphyrin (e.g., etioporphyrin, mesoporphyrin, protoporphyrin (e.g., heme or hematin), coproporphyrin, or the like), a nucleoside (e.g., adenine, guanine, cytosine, thymine, uracil), a nucleotide (e.g., AMP, ADP, ATP, GMP, GDP, GTP, CMP, CDP, CTP, TMP, TDP, TTP, UMP, UDP, UTP), a glycerol phosphate, a biotin (e.g., biotin or carboxybiotin), a pyridoxal (e.g., pyridoxal phosphate, pyridoxal, pyridoxamine, pyridoxamine phosphate, or Schiff's bases thereof), an oxoglutaric acid (e.g., 2-oxoglutarate), a coenzyme A, a carnitine, a folic acid (e.g., tetrahydrofolic acid, 5-formyltetrahydrofolic acid, 10-formyltetrahydrofolic acid, 5,10-methenyltetrahydrofolic acid, 5,10-methylenetetrahydrofolic acid, 5-hydroxymethyltetrahydrofolic acid, 5-formiminotetrahydrofolic acid, or the like), an adenosylhomocysteine, a cobamide coenzyme (e.g., coenzyme B₁₂, a cobalamin such as methyl-cobalamin, or the like), adenosine 3′,5′-bisphosphate, thiamin diphosphate, ferritin, salt thereof, a mixture or combination thereof, or the like.

In an embodiment, the present recognition element can be or include a lipophilic moiety. Suitable lipophilic moieties include one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like.

Suitable recognition elements include carboxylic acids (e.g., mono and di-carboxylates) with the carboxylate appended to a lipophilic moiety, such as one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); or the like. Such carboxylic acids include arachidonic acid, linoleic acid, linolenic acid, oleic acid, and the like. Such carboxylic acids can be immobilized on a support through covalent bonding or electrostatic interaction between.

Suitable recognition elements include carboxylic acids (e.g., mono and di-carboxylates) with the carboxylate appended to a an organic radical, such as one or more branched or straight chain C₂₋₈ alkyl, arylalkyl, alkenyl, alkynyl, or the like. These carboxylic acids can include substituted aryl moieties (e.g., phenyl or naphthyl moieties). Such carboxylic acids include acetic acid, propionic acid, butanoic acid, pentanoic acid, hexanoic acid, heptanoic acid, octanoic acid, oxalic acid, malonic acid, succinic acid, glutaric acid, adipic acid, pimelic acid, benzoic acid, and the like. Such carboxylic acids can be immobilized on a support through covalent bonding or electrostatic interaction between the carboxyl(ate) and the support or lawn.

In an embodiment, the recognition element is or includes an amino acid. Suitable amino acids include a natural or synthetic amino acid. Amino acids include carboxyl and amine functional groups. In their side chains, amino acids can also include a moiety with one or more of positive charge, negative charge, acid, base, electron acceptor, electron donor, hydrogen bond donor, hydrogen bond acceptor, free electron pair, π electrons, charge polarization, hydrophilicity, or hydrophobicity. Suitable amino acids include those with a functional group on the side chain. The side chain functional group can include, for natural amino acids, an amine (e.g., alkyl amine, heteroaryl amine), hydroxyl, phenol, carboxyl, thiol, thioether, or amidino group.

Any of the natural amino acids can be employed as a recognition element. The natural amino acids include aliphatic amino acids (e.g., alanine, valine, leucine, and isoleucine), hydroxyamino acids (e.g., serine, threonine, and tyrosine), dicarboxylic acids (e.g., glutamic acid and aspartic acid), amides (e.g., glutamine and asparagine), amino acids with basic sidechains (e.g., lysine, hydroxylysine, histidine, and arginine), aromatic amino acids (e.g., histidine, phenylalanine, tyrosine, tryptophan, and thyroxine), sulfur containing amino acids (e.g., cysteine, cystine, and methionine), imino acids (e.g., proline and hydroxyproline). Natural amino acids suitable for use as recognition elements include, for example, serine, threonine, tyrosine, aspartic acid, glutamic acid, asparagine, glutamine, cysteine, lysine, arginine, histidine.

Synthetic amino acids can include the naturally occurring side chain functional groups or synthetic side chain functional groups which modify or extend the natural amino acids with alkyl, substituted alkyl, cycloalkyl, heterocyclic, substituted heterocyclic, aryl alkyl, aryl, heteroaryl, heteroaryl alkyl, and like moieties as framework and with carboxyl, amine, hydroxyl, phenol, carbonyl, or thiol functional groups. Preferred synthetic amino acids include β-amino acids and homo or β analogs of natural amino acids.

In an embodiment, the building block is or includes a dipeptide. Any of the 400 dipeptides including the 20 natural amino acids in any order can be employed as building blocks. Suitable dipeptides include muramyl dipeptide or the like.

In an embodiment the recognition element can be or include a therapeutic or pharmacologically active agent. Suitable therapeutic or pharmacologically active agents include a nitrate, nitric oxide, a nitric oxide promoter, nitric oxide donors, dipyridamole, or another vasodilator; HYTRIN® or another antihypertensive agent; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; a retinoid or another antisecretory agent; cytochalasin or another actin inhibitor; methotrexate or another antimetabolite or antiproliferative agent; tamoxifen citrate, TAXOL®, paclitaxel, or derivatives thereof, rapamycin, vinblastine, vincristine, vinorelbine, etoposide, tenopiside, dactinomycin (actinomycin D), daunorubicin, doxorubicin, idarubicin, an anthracycline, mitoxantrone, bleomycin, plicamycin (mithramycin), mitomycin, mechlorethamine, cyclophosphamide and its analogs, chlorambucil, an ethylenimine, a methylmelamine, an alkyl sulfonate (e.g., busulfan), a nitrosourea (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, or other anti-cancer chemotherapeutic agents; cyclosporin, tacrolimus (FK-506), azathioprine, mycophenolate mofetil, mTOR inhibitors, or another immunosuppressive agent; cortisol, cortisone, dexamethasone, dexamethasone sodium phosphate, dexamethasone acetate, a dexamethasone derivative, betamethasone, fludrocortisone, prednisone, prednisolone, 6U-methylprednisolone, triamcinolone (e.g., triamcinolone acetonide), or another steroidal agent; trapidil (a PDGF antagonist); dopamine, bromocriptine mesylate, pergolide mesylate, or another dopamine agonist; captopril, enalapril or another angiotensin converting enzyme (ACE) inhibitor; angiotensin receptor blockers; ascorbic acid, alpha tocopherol, deferoxamine, a 21-aminosteroid (lasaroid) or another free radical scavenger, iron chelator or antioxidant; estrogen or another sex hormone; AZT or another antipolymerase; acyclovir, famciclovir, rimantadine hydrochloride, ganciclovir sodium, Norvir, Crixivan, α-methyl-1-adamantanemethylamine, hydroxy-ethoxymethylguanine, adamantanamine, 5-iodo-2′-deoxyuridine, trifluorothymidine, adenine arabinoside, or another antiviral agent; 5-aminolevulinic acid, meta-tetrahydroxyphenylchlorin, hexadecafluorozinc phthalocyanine, tetramethyl hematoporphyrin, rhodamine 123 or other photodynamic therapy agents; PROSCAR®, HYTRIN® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these.

In an embodiment, the recognition element can be or can include an antibiotic. Examples of antibiotics include penicillin, tetracycline, chloramphenicol, minocycline, doxycycline, vancomycin, bacitracin, kanamycin, neomycin, gentamycin, erythromycin and cephalosporins. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

In an embodiment, the recognition element can be or can include an enzyme inhibitor. Suitable enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatecho-1, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, allopurinol, and the like.

In an embodiment, the recognition element is or includes a signal element that produces a detectable signal when a test ligand is bound to the receptor. In an embodiment, the signal element can produce an optical signal or a electrochemical signal. Suitable optical signals include chemiluminescence or fluorescence. The signal element can be a fluorescent moiety. The fluorescent molecule can be one that is quenched by binding to the artificial receptor. For example, the signal element can be a molecule that fluoresces only when binding occurs. Suitable electrochemical signal elements include those that give rise to current or a potential. Suitable electrochemical signal elements include phenols and anilines, such as those with substitutents oriented ortho or para to one another, polynuclear aromatic hydrocarbons, sulfide-disulfide, sulfide-sulfoxide-sulfone, polyenes, polyeneynes, and the like. Suitable electrochemical signal elements include quinones and ferrocenes.

Linkers

The linker is selected to provide a suitable coupling of the building block to a support. The framework can interact with the ligand as part of the artificial receptor. The linker can also provide bulk, distance from the support, hydrophobicity, hydrophilicity, and like structural characteristics to the building block. Coupling building blocks to the support can employ covalent bonding or noncovalent interactions. Suitable noncovalent interactions include interactions between ions, hydrogen bonding, van der Waals interactions, and the like. In an embodiment, the linker includes moieties that can engage in covalent bonding or noncovalent interactions. In an embodiment, the linker includes moieties that can engage in covalent bonding. Suitable groups for forming covalent and reversible covalent bonds are described hereinabove.

Linkers for Reversibly Immobilizable Building Blocks

The linker can be selected to provide suitable reversible immobilization of the building block on a support or lawn. In an embodiment, the linker forms a covalent bond with a functional group on the framework. In an embodiment, the linker also includes a functional group that can reversibly interact with the support or lawn, e.g., through reversible covalent bonding or noncovalent interactions.

In an embodiment, the linker includes one or more moieties that can engage in reversible covalent bonding. Suitable groups for reversible covalent bonding include those described hereinabove. An artificial receptor can include building blocks reversibly immobilized on the lawn or support through, for example, imine, acetal, ketal, disulfide, ester, or like linkages. Such functional groups can engage in reversible covalent bonding. Such a functional group can be referred to as a covalent bonding moiety, e.g., a second covalent bonding moiety.

In an embodiment, the linker can be functionalized with moieties that can engage in noncovalent interactions. For example, the linker can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like.

In an embodiment, the present methods and compositions can employ a linker including a charged moiety (e.g., a second charged moiety). Suitable charged moieties include positively charged moieties and negatively charged moieties. Suitable positively charged moieties include amines, quaternary ammonium moieties, sulfonium, phosphonium, ferrocene, and the like. Suitable negatively charged moieties (e.g., at neutral pH in aqueous compositions) include carboxylates, phenols substituted with strongly electron withdrawing groups (e.g., tetrachlorophenols), phosphates, phosphonates, phosphinates, sulphates, sulphonates, thiocarboxylates, and hydroxamic acids.

In an embodiment, the present methods and compositions can employ a linker including a group that can hydrogen bond, either as donor or acceptor (e.g., a second hydrogen bonding group). For example, the linker can include one or more carboxyl groups, amine groups, hydroxyl groups, carbonyl groups, or the like. Ionic groups can also participate in hydrogen bonding.

In an embodiment, the present methods and compositions can employ a linker including a lipophilic moiety (e.g., a second lipophilic moiety). Suitable lipophilic moieties include one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. In an embodiment the linker includes or is a lipid, such as a phospholipid. In an embodiment, the lipophilic moiety includes or is a 12-carbon aliphatic moiety.

In an embodiment, the linker includes a lipophilic moiety (e.g., a second lipophilic moiety) and a covalent bonding moiety (e.g., a second covalent bonding moiety). In an embodiment, the linker includes a lipophilic moiety (e.g., a second lipophilic moiety) and a charged moiety (e.g., a second charged moiety).

In an embodiment, the linker forms or can be visualized as forming a covalent bond with an alcohol, phenol, thiol, amine, carbonyl, or like group on the framework. Between the bond to the framework and the group participating in or formed by the reversible interaction with the support or lawn, the linker can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

For example, suitable linkers can include: the functional group participating in or formed by the bond to the framework, the functional group or groups participating in or formed by the reversible interaction with the support or lawn, and a linker backbone moiety. The linker backbone moiety can include about 4 to about 48 carbon or heteroatoms, about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms, or the like. The linker backbone can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, mixtures thereof, or like moiety.

In an embodiment, the linker includes a lipophilic moiety, the functional group participating in or formed by the bond to the framework, and, optionally, one or more moieties for forming a reversible covalent bond, a hydrogen bond, or an ionic interaction. In such an embodiment, the lipophilic moiety can have about 4 to about 48 carbons, about 8 to about 14 carbons, about 12 to about 24 carbons, about 16 to about 18 carbons, or the like. In such an embodiment, the linker can include about 1 to about 8 reversible bond/interaction moieties or about 2 to about 4 reversible bond/interaction moieties. Suitable linkers have structures such as (CH₂)ₙCOOH, with n=12-24, n=17-24, or n=16-18.

### Additional Embodiments of Linkers

The linker can be selected to provide a suitable covalent coupling of the building block to a support. The framework can interact with the ligand as part of the artificial receptor. The linker can also provide bulk, distance from the support, hydrophobicity, hydrophilicity, and like structural characteristics to the building block. In an embodiment, the linker forms a covalent bond with a functional group on the framework. In an embodiment, before attachment to the support the linker also includes a functional group that can be activated to react with or that will react with a functional group on the support. In an embodiment, once attached to the support, the linker forms a covalent bond with the support and with the framework.

In an embodiment, the linker forms or can be visualized as forming a covalent bond with an alcohol, phenol, thiol, amine, carbonyl, or like group on the framework. The linker can include a carboxyl, alcohol, phenol, thiol, amine, carbonyl, maleimide, or like group that can react with or be activated to react with the support. Between the bond to the framework and the group formed by the attachment to the support, the linker can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

The linker can include a good leaving group bonded to, for example, an alkyl or aryl group. The leaving group being “good” enough to be displaced by the alcohol, phenol, thiol, amine, carbonyl, or like group on the framework. Such a linker can include a moiety represented by the formula: R—X, in which X is a leaving group such as halogen (e.g., —Cl, —Br or —I), tosylate, mesylate, triflate, and R is alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

Suitable linker groups include those of formula: (CH₂)ₙCOOH, with n=1-16, n=2-8, n=2-6, or n=3. Reagents that form suitable linkers are commercially available and include any of a variety of reagents with orthogonal functionality.

### Additional Embodiments of Building Blocks

In an embodiment, building blocks can be represented by Formula 2: 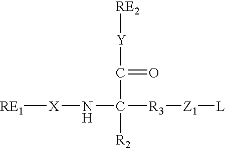
in which: RE₁ is recognition element 1, RE₂ is recognition element 2, and L is a linker. X is absent, C═O, CH₂, NR, NR₂, NH, NHCONH, SCONH, CH═N, or OCH₂NH. In certain embodiments, X is absent or C═O. Y is absent, NH, O, CH₂, or NRCO. In certain embodiments, Y is NH or O. In an embodiment, Y is NH. Z₁ and Z₂ can independently be CH2, O, NH, S, CO, NR, NR₂, NHCONH, SCONH, CH═N, or OCH₂NH. In an embodiment, Z₁ and/or Z₂ can independently be O. Z₂ is optional. R₂ is H, CH₃, or another group that confers chirality on the building block and has size similar to or smaller than a methyl group. R₃ is CH₂; CH₂-phenyl; CHCH₃; (CH₂)ₙ with n=2-3; or cyclic alkyl with 3-8 carbons, e.g., 5-6 carbons, phenyl, naphthyl. In certain embodiments, R₃ is CH₂ or CH₂-phenyl.

RE₁ is B1, B2, B3, B3a, B4, B5, B6, B7, B8, B9, A1, A2, A3, A3a, A4, A5, A6, A7, A8, or A9. In certain embodiments, RE₁ is B1, B2, B3, B3a, B4, B5, B6, B7, B8, or B9. RE₂ is A1, A2, A3, A3a, A4, A5, A6, A7, A8, A9, B1, B2, B3, B3a, B4, B5, B6, B7, B8, or B9. In certain embodiments, RE₂ is A1, A2, A3, A3a, A4, A5, A6, A7, A8, or A9. In an embodiment, RE₁ can be B2, B3a, B4, B5, B6, B7, or B8. In an embodiment, RE₂ can be A2, A3a, A4, A5, A6, A7, or A8.

In an embodiment, L is the functional group participating in or formed by the bond to the framework (such groups are described herein), the functional group or groups participating in or formed by the reversible interaction with the support or lawn (such groups are described herein), and a linker backbone moiety. In an embodiment, the linker backbone moiety is about 4 to about 48 carbon or heteroatom alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or mixtures thereof; or about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms.

In an embodiment, the L is the functional group participating in or formed by the bond to the framework (such groups are described herein) and a lipophilic moiety (such groups are described herein) of about 4 to about 48 carbons, about 8 to about 14 carbons, about 12 to about 24 carbons, about 16 to about 18 carbons. In an embodiment, this L also includes about 1 to about 8 reversible bond/interaction moieties (such groups are described herein) or about 2 to about 4 reversible bond/interaction moieties. In an embodiment, L is (CH₂)ₙCOOH, with n=12-24, n=17-24, or n=16-18.

In an embodiment, L is (CH₂)ₙCOOH, with n=1-16, n=2-8, n=4-6, or n=3.

Building blocks including an A and/or a B recognition element, a linker, and an amino acid framework can be made by methods illustrated in general Scheme 1.

Building Blocks Including a Tether

A building block can be visualized as including several components, such as one or more frameworks, one or more linkers, one or more recognition elements, and/or one or more tethers. The framework can be covalently coupled to each of the other building block components. The linker can be covalently coupled to the framework. The linker can be coupled to a support through one or more of covalent, electrostatic, hydrogen bonding, van der Waals, or like interactions. The recognition element can be covalently coupled to the framework. The tether can be covalently coupled to the linker and to the framework.

In an embodiment, a building block includes a framework, a linker, a recognition element, and a tether. In an embodiment, a building block includes a framework, a linker, a tether, and two recognition elements. The framework can be selected for functional groups that provide for coupling to the recognition moiety and for coupling to or being the tether and/or linking moieties.

In an embodiment, the present invention relates to a building block including a tether moiety. The tether can include the framework. The tether moiety can provide spacing or distance between the recognition element and the support or scaffold to which the building block is immobilized. A tether moiety can have any of a variety of characteristics or properties including flexibility, rigidity or stiffness, ability to bond to another tether moiety, and the like. The tether moiety can include the linker. The framework moiety be envisioned as forming all or part of the tether moiety.

Suitable tether moieties can include a polyethylene glycol, a polyamide, a linear polymer, a peptide, a polypeptide, an oligosaccharide, a polysaccharide, a semifunctionalized oligo- or polyglycine. In an embodiment, the tether is or includes a polymer of up to 2000 carbon atoms (e.g., up to 48 carbon atoms). Such a polymer can be naturally occurring or synthetic. Suitable polymers include a polyether or like polymer, such as a PEG, a polyethyleneimine, polyacrylate (e.g., substituted polyacrylates), salt thereof, a mixture or combination thereof, or the like. Suitable PEGs include PEG 1500 up to PEG 20,000, for example, PEG 1450, PEG 3350, PEG 4500, PEG 8000, PEG 20,000, and the like.

Suitable tether moieties can include one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. In an embodiment, the lipophilic moiety includes or is a 12-carbon aliphatic moiety.

Rigid tether moieties can include conformationally restricted groups such as imines, aromatics, and polyaromatics. Rigid tether moieties can include one or more branched or straight chain C₆₋₃₆ alkenyl, C₈₋₂₄ alkenyl, C₁₂₋₂₄ alkenyl, C₁₂₋₁₈ alkenyl, or the like, with, for example, 2 to 8 double bonds; C₆₋₃₆ alkynyl, C₈₋₂₄ alkynyl, C₁₂₋₂₄ alkynyl, C₁₂₋₁₈ alkynyl, or the like, with, for example, 1 to 8 triple bonds; chains with 3-8 double or triple bonds; chains including aryl or substituted aryl moieties (e.g., phenyl or naphthyl moieties at the end or middle of a chain); polyaromatic hydrocarbon moieties; and the like. The alkenyl or alkynyl group can include branching; within chain functionality like an ether group; terminal functionality like alcohol, amide, carboxylate or the like; or the like. Rigid tether moieties can include a steroid moiety, such as cholesterol, a corrin or another porphyrin, a polynuclear aromatic moiety, a polar polymer fixed with metal ions, or the like.

In an embodiment, a rigid tether moiety can include more than one tether moiety. For example, a rigid tether moiety can include a plurality of hydrophobic chains, such as those described in the paragraph above and in the paragraph below. The hydrophobic chains if held in sufficient proximity on the support or scaffold will, in a hydrophobic solvent, form a grouping sufficiently rigid to hold one or more sets of recognition elements in place. In another embodiment, a rigid tether moiety can include a plurality of otherwise flexible tether moieties crosslinked to one another. The crosslinking can include, for example, covalent bonding, electrostatic interactions, hydrogen bonding, or hydrophobic interactions. Groups for forming such interactions are disclosed herein.

Flexible tether moieties can include one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 2 double bonds; C₆₋₃₆ alkynyl, C₈₋₂₄ alkynyl, C₁₂₋₂₄ alkynyl, C₁₂-18 alkynyl, or the like, with, for example, 1 to 2 triple bonds; chains with 1-2 double or triple bonds; chains including 1 to 2 aryl or substituted aryl moieties (e.g., phenyl or naphthyl moieties at the end or middle of a chain); cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. In an embodiment, the lipophilic moiety includes or is a 12-carbon aliphatic moiety.

In an embodiment, the tether forms or can be visualized as forming a covalent bond with an alcohol, phenol, thiol, amine, carbonyl, or like group on the framework. Between the bond to the framework and the group participating in or formed by the interaction with the support or lawn, the linker can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

Suitable tethers can include, for example: the functional group participating in or formed by the bond to the framework, the functional group or groups participating in or formed by the interaction with the support or lawn, and a tether backbone moiety. The tether backbone moiety can include about 8 to about 200 carbon or heteroatoms, about 12 to about 150 carbon or heteroatoms, about 16 to about 100 carbon or heteroatoms, about 16 to about 50 carbon or heteroatoms, or the like. The tether backbone can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, mixtures thereof, or like moiety. Suitable tethers have structures such as (CH₂)ₙCOOH, with n=12-24, n=17-24, or n=16-18.

The tether can interact with the ligand as part of the artificial receptor. The tether can also provide bulk, distance from the support, hydrophobicity, hydrophilicity, and like structural characteristics to the building block. In an embodiment, the tether forms a covalent bond with a functional group on the framework. In an embodiment, the tether also includes a functional group that can couple to the tether or to the support or lawn, e.g., through covalent bonding or noncovalent interactions.

In an embodiment, the tether includes one or more moieties for forming a reversible covalent bond, a hydrogen bond, or an ionic interaction, e.g., with another tether moiety. For example, the linker can include about 1 to about 20 reversible bond/interaction moieties or about 2 to about 10 reversible bond/interaction moieties.

In an embodiment, the tether includes one or more moieties that can engage in reversible covalent bonding. Suitable groups for reversible covalent bonding include those described hereinabove. Such groups for reversible covalent bonds can be part of links between tether moieties. The tether-tether links can include, for example, imine, acetal, ketal, disulfide, ester, or like linkages. Such functional groups can engage in reversible covalent bonding. Such a functional group can be referred to as a covalent bonding moiety.

In an embodiment, the tether can be functionalized with moieties that can engage in noncovalent interactions. For example, the tether can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like.

In an embodiment, the present methods and compositions can employ a tether including a charged moiety. Suitable charged moieties include positively charged moieties and negatively charged moieties. Suitable positively charged moieties include protonated amines, quaternary ammonium moieties, sulfonium, sulfoxonium, phosphonium, ferrocene, and the like. Suitable negatively charged moieties (e.g., at neutral pH in aqueous compositions) include carboxylates, phenols substituted with strongly electron withdrawing groups (e.g., tetrachlorophenols), phosphates, phosphonates, phosphinates, sulphates, sulphonates, thiocarboxylates, and hydroxamic acids.

In an embodiment, the present methods and compositions can employ a tether including a group that can hydrogen bond, either as donor or acceptor (e.g., a second hydrogen bonding group). For example, the tether can include one or more carboxyl groups, amine groups, hydroxyl groups, carbonyl groups, or the like. Ionic groups can also participate in hydrogen bonding.

In an embodiment, building blocks can be represented by Formula 3: 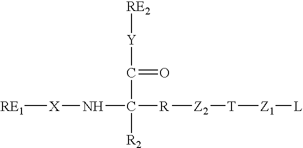
in which: RE₁ is recognition element 1, RE₂ is recognition element 2, T is an optional tether, and L is a linker. X is absent, C═O, CH₂, NR, NR₂, NH, NHCONH, SCONH, CH═N, or OCH₂NH. In certain embodiments, X is absent or C═O. Y is absent, NH, O, CH₂, or NRCO. In certain embodiments, Y is NH or O. In an embodiment, Y is NH. Z₁ and Z₂ can independently be CH₂, O, NH, S, CO, NR, NR₂, NHCONH, SCONH, CH═N, or OCH₂NH. In an embodiment, Z₁ and/or Z₂ can independently be O. Z₂ is optional. R₂ is H, CH₃, or another group that confers chirality on the building block and has size similar to or smaller than a methyl group. R₃ is CH₂; CH₂-phenyl; CHCH₃; (CH₂)ₙ with n=2-3; or cyclic alkyl with 3-8 carbons, e.g., 5-6 carbons, phenyl, naphthyl. In certain embodiments, R₃ is CH₂ or CH₂-phenyl.

RE₁ is B1, B2, B3, B3a, B4, B5, B6, B7, B8, B9, A1, A2, A3, A3a, A4, A5, A6, A7, A8, or A9. In certain embodiments, RE₁ is B1, B2, B3, B3a, B4, B5, B6, B7, B8, or B9. RE₂ is A1, A2, A3, A3a, A4, A5, A6, A7, A8, A9, B1, B2, B3, B3a, B4, B5, B6, B7, B8, or B9. In certain embodiments, RE₂ is A1, A2, A3, A3a, A4, A5, A6, A7, A8, or A9. In an embodiment, RE₁ can be B2, B3a, B4, B5, B6, B7, or B8. In an embodiment, RE₂ can be A2, A3a, A4, A5, A6, A7, or A8.

T can be any of the tether moieties described hereinabove.

In an embodiment, L is the functional group participating in or formed by the bond to the framework (such groups are described herein), the functional group or groups participating in or formed by the reversible interaction with the support or lawn (such groups are described herein), and a linker backbone moiety. In an embodiment, the linker backbone moiety is about 4 to about 48 carbon or heteroatom alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or mixtures thereof; or about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms.

In an embodiment, the L is the functional group participating in or formed by the bond to the framework (such groups are described herein) and a lipophilic moiety (such groups are described herein) of about 4 to about 48 carbons, about 8 to about 14 carbons, about 12 to about 24 carbons, about 16 to about 18 carbons. In an embodiment, this L also includes about 1 to about 8 reversible bond/interaction moieties (such groups are described herein) or about 2 to about 4 reversible bond/interaction moieties. In an embodiment, L is (CH₂)ₙCOOH, with n=12-24, n=17-24, or n=16-18.

In an embodiment, L is (CH₂)ₙCOOH, with n=1-16, n=2-8, n=4-6, or n=3.

Building blocks including an A and/or a B recognition element, a linker, and an amino acid framework can be made by methods illustrated in general Scheme 1.

The present invention may be better understood with reference to the following examples. These examples are intended to be representative of specific embodiments of the invention, and are not intended as limiting the scope of the invention.

### EXAMPLES

### Example 1

### Synthesis of Building Blocks

Selected building blocks representative of the alkyl-aromatic-polar span of the an embodiment of the building blocks were synthesized and demonstrated effectiveness of these building blocks for making candidate artificial receptors. These building blocks were made on a framework that can be represented by tyrosine and included numerous recognition element pairs. These recognition element pairs include enough of the range from alkyl, to aromatic, to polar to represent a significant degree of the interactions and functional groups of the full set of 81 such building blocks.

Synthesis

Building block synthesis employed a general procedure outlined in Scheme 1, which specifically illustrates synthesis of a building block on a tyrosine framework with recognition element pair A4B4. This general procedure was employed for synthesis of building blocks including TyrA1B1 [1-1], TyrA2B2, TyrA2B4, TyrA2B6, TyrA2B8, TyrA4B2, TyrA4B4, TyrA4B6, TyrA4B8, TyrA6B2, TyrA6B4, TyrA6B6, TyrA6B8, TyrA8B2, TyrA8B4, TyrA8B6, TyrA8B8, and TyrA9B9, respectively. 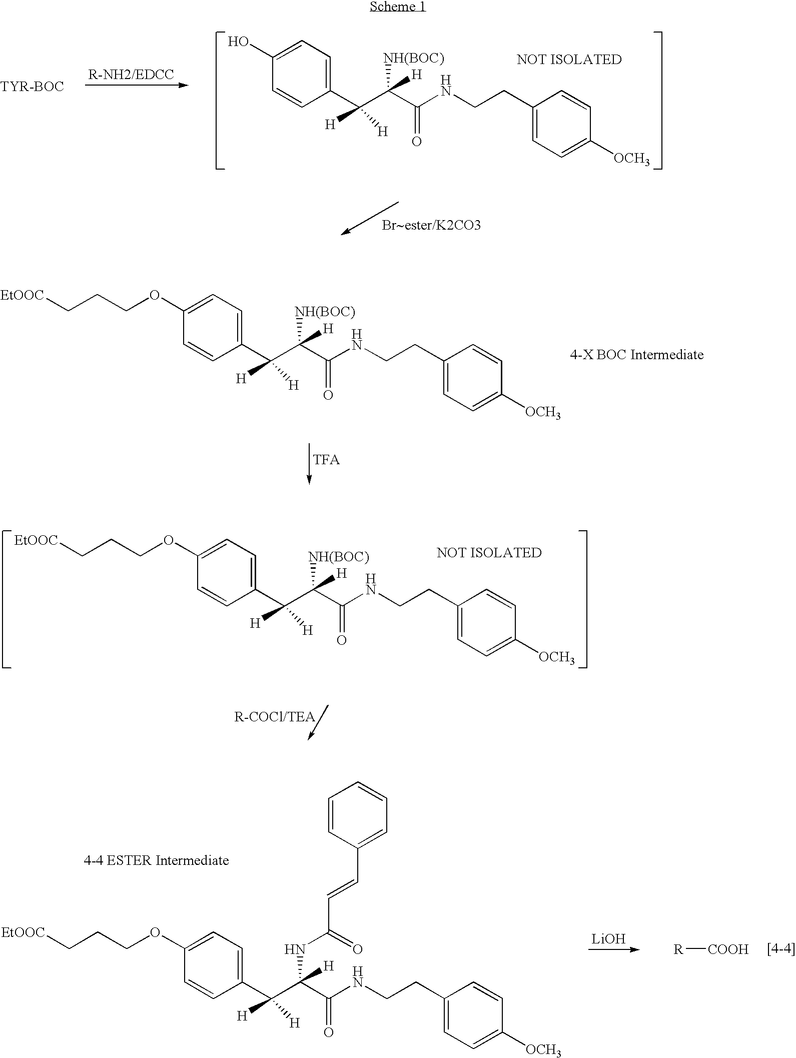
Results

Synthesis of the desired building blocks proved to be generally straightforward. These syntheses illustrate the relative simplicity of preparing the building blocks with 2 recognition elements having different structural characteristics or structures (e.g. A4B2, A6B3, etc.) once the building blocks with corresponding recognition elements (e.g. A2B2, A4B4, etc) have been prepared via their X BOC intermediate.

The conversion of one of these building blocks to a building block with a lipophilic linker can be accomplished by reacting the activated building block with, for example, dodecyl amine.

### Example 2

### Preparation and Evaluation of Microarrays of Candidate Artificial Receptors

Microarrays of candidate artificial receptors were made and evaluated for binding several protein ligands. The results obtained demonstrate the 1) the simplicity with which microarrays of candidate artificial receptors can be prepared, 2) binding affinity and binding pattern reproducibility, 3) significantly improved binding for building block heterogeneous receptor environments when compared to the respective homogeneous controls, and 4) ligand distinctive binding patterns (e.g., working receptor complexes).

Materials and Methods

Building blocks were synthesized and activated as described in Example 1. The building blocks employed in this example were TyrA1B1 [1-1], TyrA2B2, TyrA2B4, TyrA2B6, TyrA4B2, TyrA4B4, TyrA4B6, TyrA6B2, TyrA6B4, and TyrA6B6. The abbreviation for the building block including a linker, a tyrosine framework, and recognition elements AxBy is TyrAxBy.

Microarrays for the evaluation of the 130 n=2 and n=3, and for evaluation of the 273 n=2, n=3, and n=4, candidate receptor environments were prepared as follows by modifications of known methods. As used herein, “n” is the number of different building blocks employed in a receptor environment. Briefly: Amine modified (amine “lawn”; SuperAmine Microarray plates) microarray plates were purchased from Telechem Inc., Sunnyvale, Calif. (www.arrayit.com). These plates were manufactured specifically for microarray preparation and had a nominal amine load of 2-4 amines per square nm according to the manufacturer. The CAM microarrays were prepared using a pin microarray spotter instrument from Telechem Inc. (SpotBot™ Arrayer) typically with 200 um diameter spotting pins from Telechem Inc. (Stealth Micro Spotting Pins, SMP6) and 400-420 um spot spacing.

The 9 building blocks were activated in aqueous dimethylformamide (DMF) solution as described above. For preparing the 384-well feed plate, the activated building block solutions were diluted 10-fold with a solution of DMF/H₂O/PEG400 (90/10/10, v/v/v; PEG400 is polyethylene glycol nominal 400 FW, Aldrich Chemical Co., Milwaukee, Wis.). These stock solutions were aliquotted (10 μl per aliquot) into the wells of a 384-well microwell plate (Telechem Inc.). A separate series of controls were prepared by aliquotting 10 μl of building block with either 10 μl or 20 μl of the activated [1-1] solution. The plate was covered with aluminum foil and placed on the bed of a rotary shaker for 15 minutes at 1,000 RPM. This master plate was stored covered with aluminum foil at −20° C. when not in use.

For preparing the 384-well SpotBot™ plate, a well-to-well transfer (e.g. A-1 to A-1, A-2 to A-2, etc.) from the feed plate to a second 384-well plate was performed using a 4 μl transfer pipette. This plate was stored tightly covered with aluminum foil at −20° C. when not in use. The SpotBot™ was used to prepare up to 13 microarray plates per run using the 4 μl microwell plate. The SpotBot™ was programmed to spot from each microwell in quadruplicate. The wash station on the SpotBot™ used a wash solution of EtOH/H₂O (20/80, v/v). This wash solution was also used to rinse the microarrays on completion of the SpotBot™ printing run. The plates were given a final rinse with deionized (DI) water, dried using a stream of compressed air, and stored at room temperature.

Certain of the microarrays were further modified by reacting the remaining amines with succinic anhydride to form a carboxylate lawn in place of the amine lawn.

The following test ligands and labels were used in these experiments:

1) r-Phycoerythrin, a commercially available and intrinsically fluorescent protein with a FW of 2,000,000.

2) Ovalbumin labeled with the Alexa™ fluorophore (Molecular Probes Inc., Eugene, Oreg.).

3) BSA, bovine serum albumin, labeled with activated Rhodamine (Pierce Chemical, Rockford, Ill.) using the known activated carboxylprotocol. BSA has a FW of 68,000; the material used for this study had ca. 1.0 rhodamine per BSA.

4) Horseradish peroxidase (HRP) modified with extra amines and labeled as the acetamide derivative or with a 2,3,7,8-tetrachlorodibenzodixoin derivative were available through known methods. Fluorescence detection of these HRP conjugates was based on the Alexa 647-tyramide kit available from Molecular Probes, Eugene, Oreg.

5) Cholera toxin labeled with the Alexa™ fluorophore (Molecular Probes Inc., Eugene, Oreg.).

Microarray incubation and analysis was conducted as follows: For test ligand incubation with the microarrays, solutions (e.g. 50 μl) of the target proteins in PBS-T (PBS with 20 μl/L of Tween-20) at typical concentrations of 10, 1.0 and 0.1 μg/ml were placed onto the surface of a microarray and allowed to react for, e.g., 30 minutes. The microarray was rinsed with PBS-T and DI water and dried using a stream of compressed air.

The incubated microarray was scanned using an Axon Model 4200A Fluorescence Microarray Scanner (Axon Instruments, Union City, Calif.). The Axon scanner and its associated software produce a false color 16-bit image of the fluorescence intensity of the plate. This 16-bit data is integrated using the Axon software to give a Fluorescence Units value (range 0-65,536) for each spot on the microarray. This data is then exported into an Excel file (Microsoft) for further analysis including mean, standard deviation and coefficient of variation calculations.

Results

The CARA™: Combinatorial Artificial Receptor Array™ concept has been demonstrated using a microarray format. A CARA microarray based on N=9 building blocks was prepared and evaluated for binding to several protein and substituted protein ligands. This microarray included 144 candidate receptors (18 n=1 controls plus 6 blanks; 36 n=2 candidate receptors; 84 n=3 candidate receptors). This microarray demonstrated: 1) the simplicity of CARA microarray preparation, 2) binding affinity and binding pattern reproducibility, 3) significantly improved binding for building block heterogeneous receptor environments when compared to the respective homogeneous controls, and 4) ligand distinctive binding patterns.

Reading the Arrays

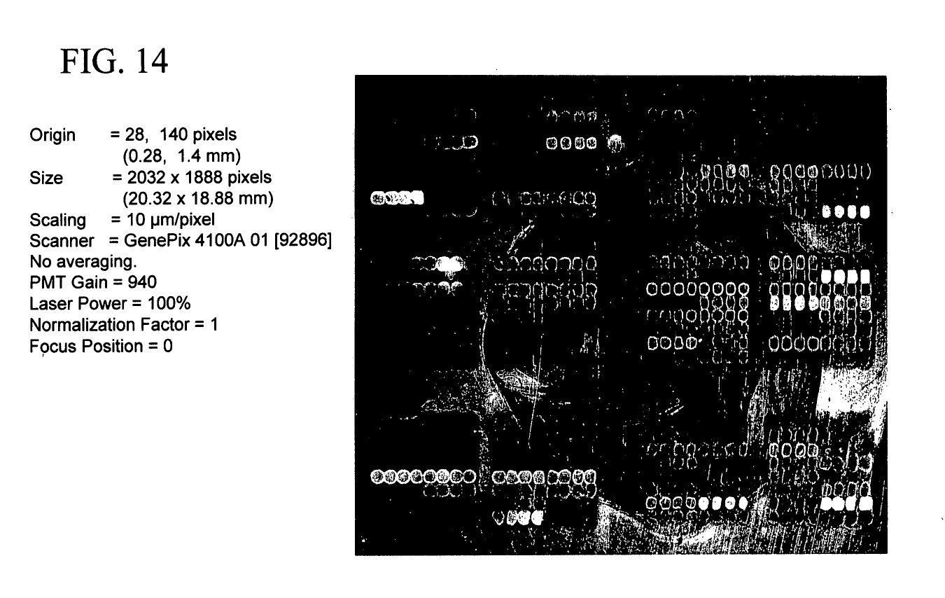

A typical false color/gray scale image of a microarray that was incubated with 2.0 μg/ml r-phycoerythrin is shown in FIG. 14. This image illustrates that the processes of both preparing the microarray and probing it with a protein test ligand produced the expected range of binding as seen in the visual range of relative fluorescence from dark to bright spots.

The starting point in analysis of the data was to take the integrated fluorescence units data for the array of spots and normalize to the observed value for the [1-1] building block control. Subsequent analysis included mean, standard deviation and coefficient of variation calculations. Additionally, control values for homogeneous building blocks were obtained from the building block plus [1-1] data.

First Set of Experiments

The following protein ligands were evaluated for binding to the candidate artificial receptors in the microarray. The resulting Fluorescence Units versus candidate receptor environment data is presented in both a 2D format where the candidate receptors are placed along the X-axis and the Fluorescence Units are shown on the Y-axis and a 3D format where the Candidate Receptors are placed in an X-Y format and the Fluorescence Units are shown on the Z-axis. A key for the composition of each spot was developed (not shown). A key for the building blocks in each of the 2D and 3D representations of the results was also developed (not shown). The data presented are for 1-2 μg/ml protein concentrations.

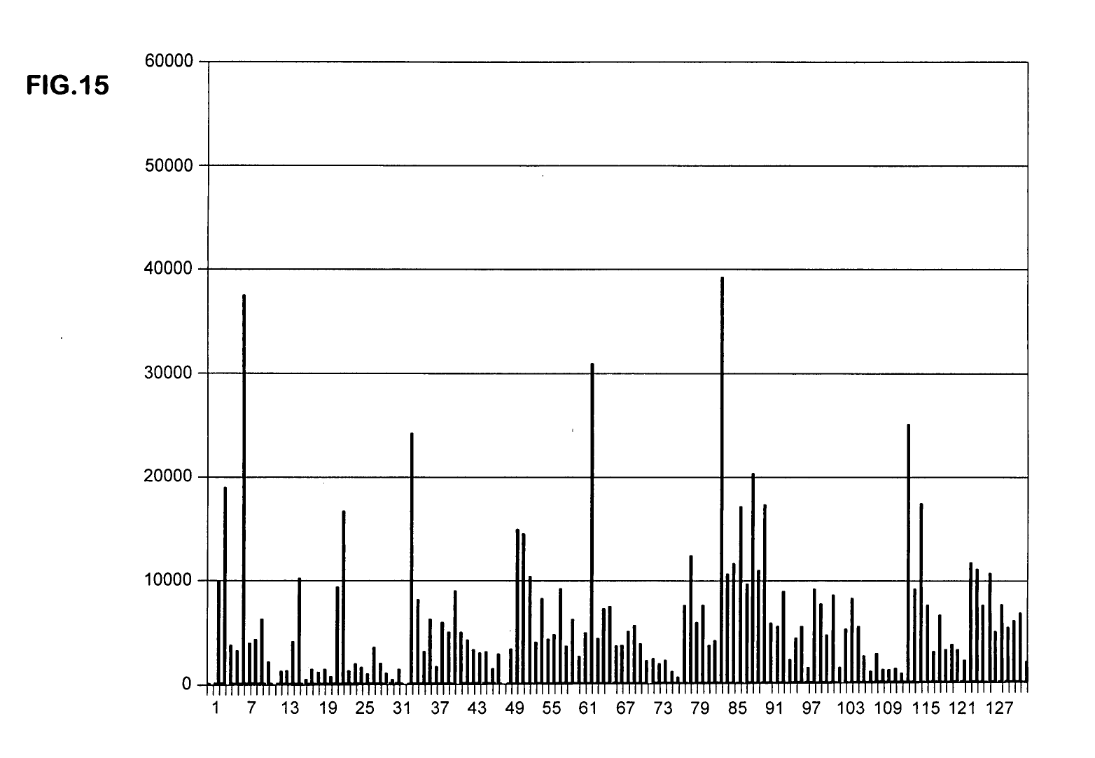

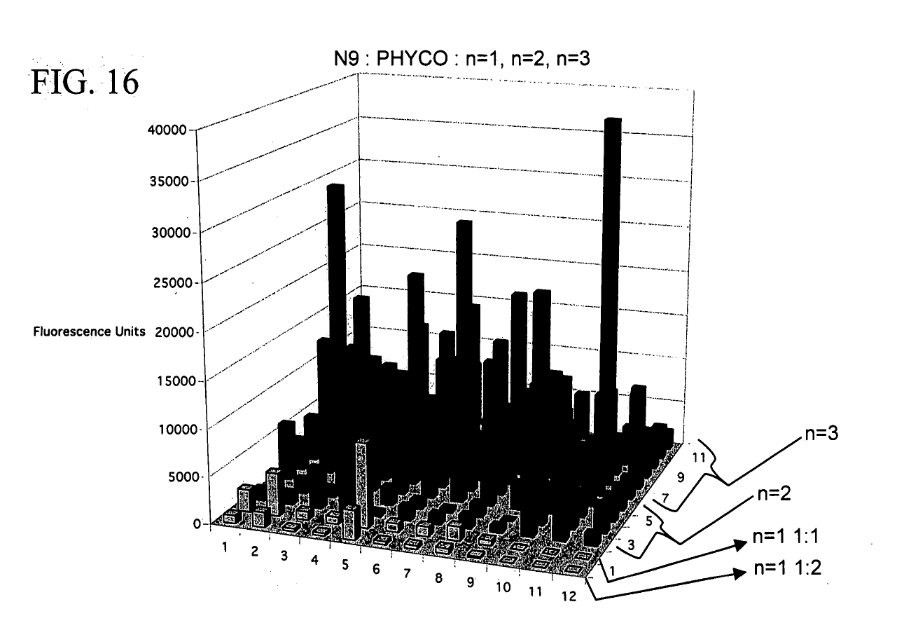

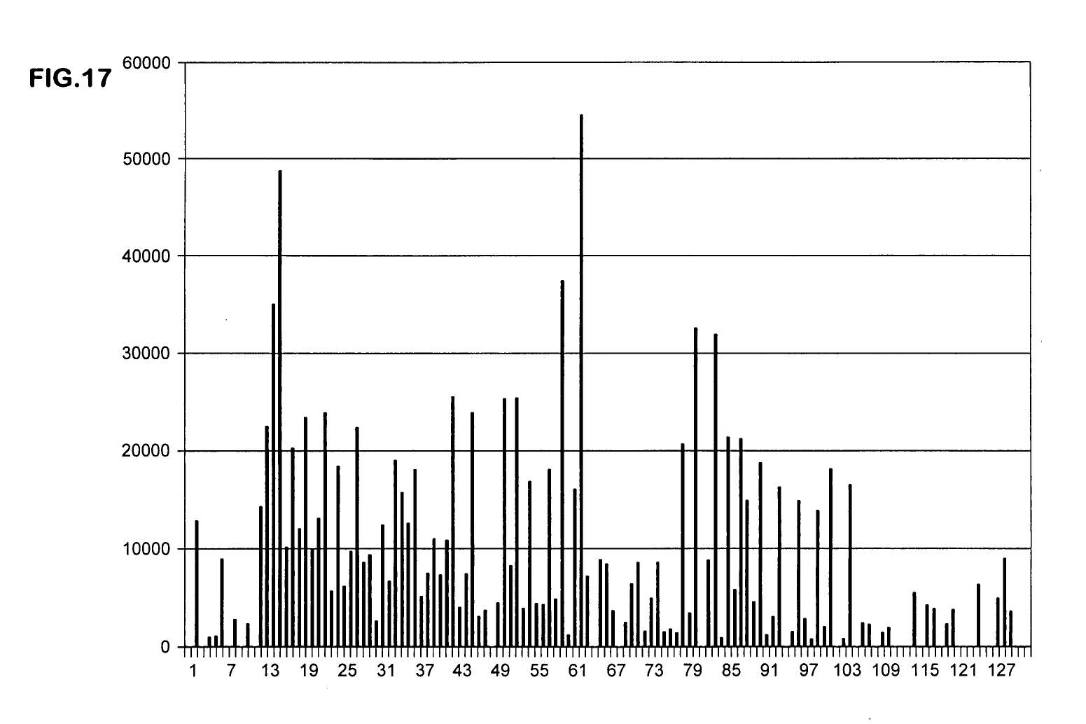

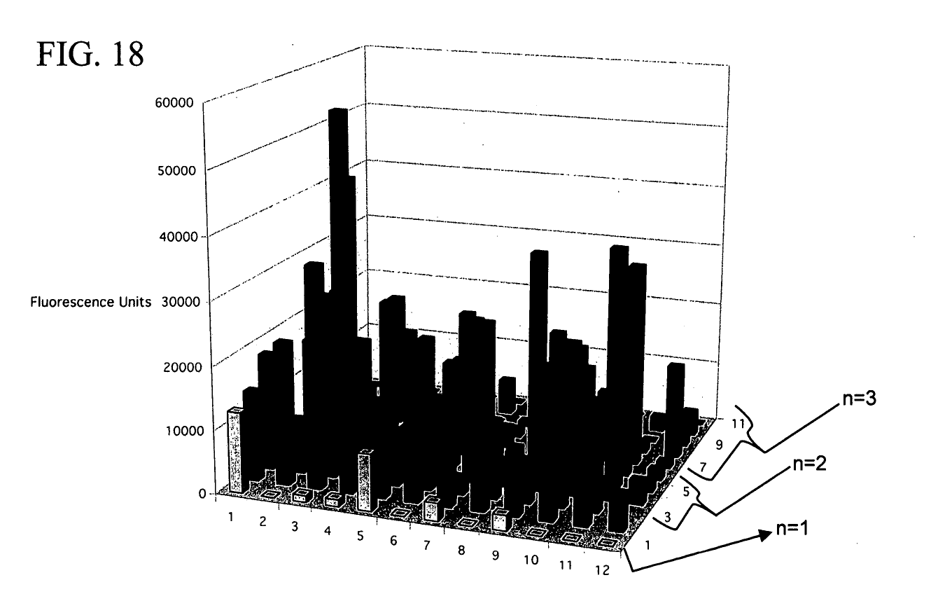

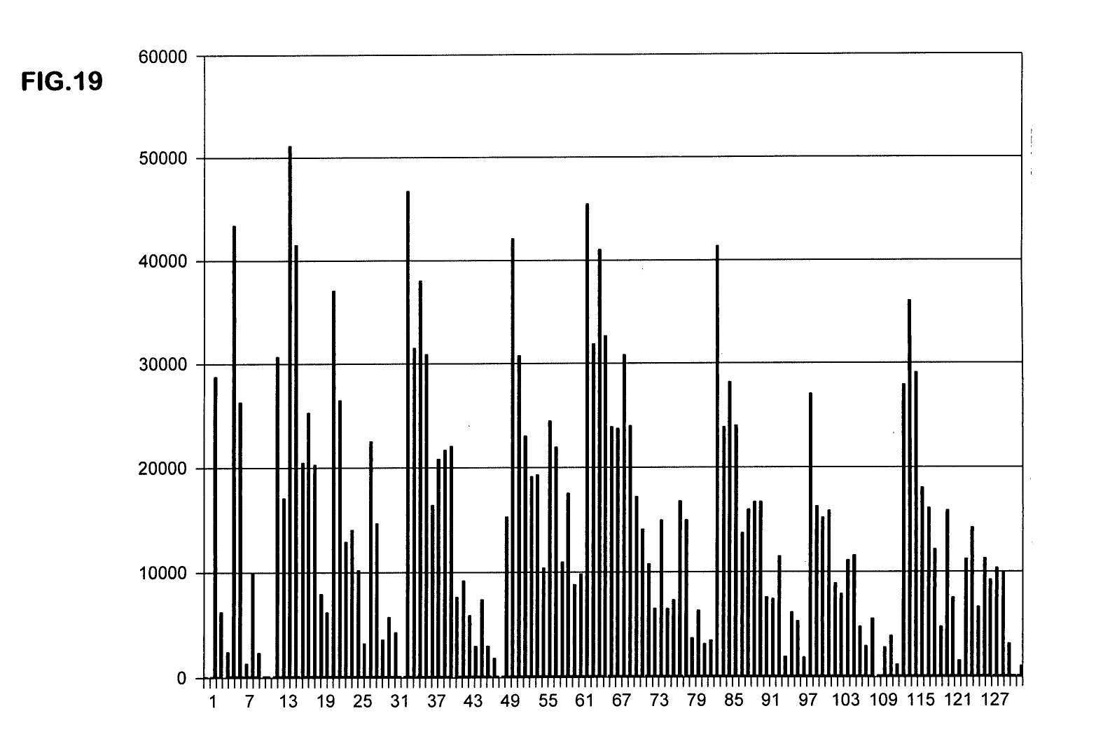

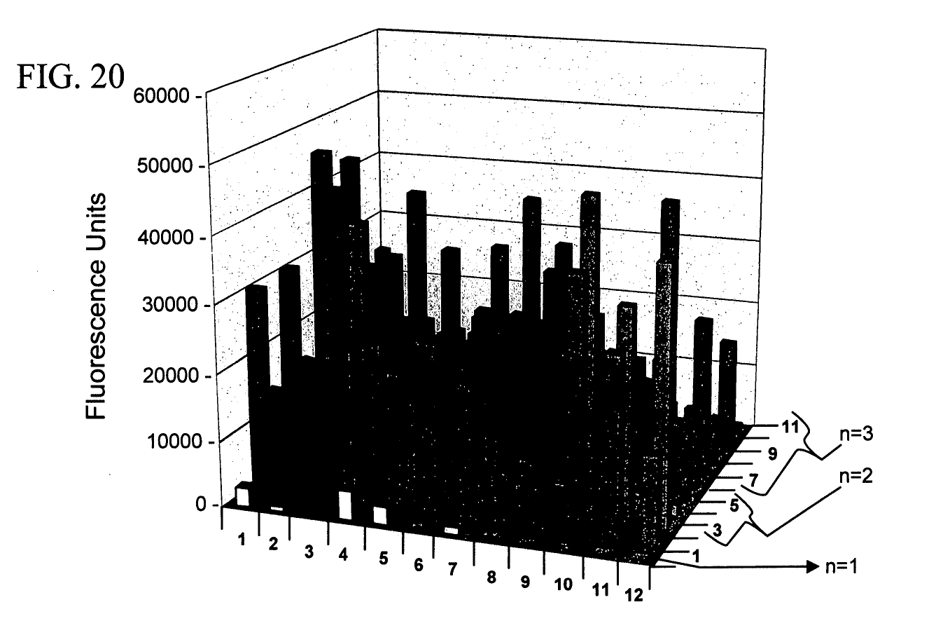

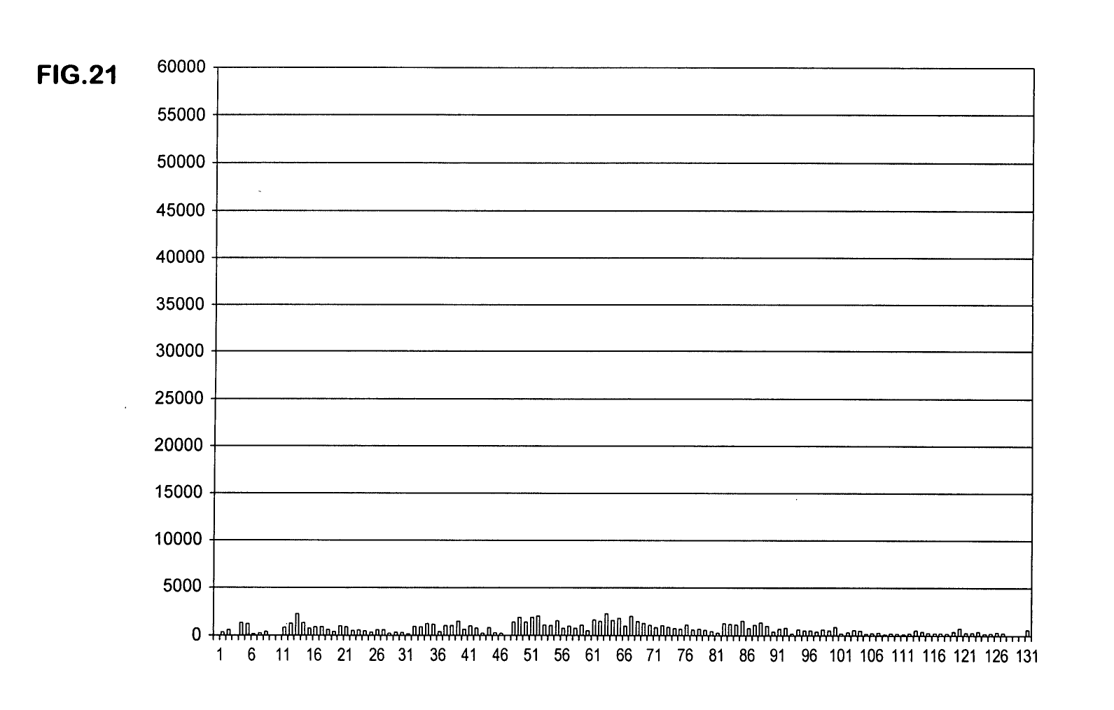

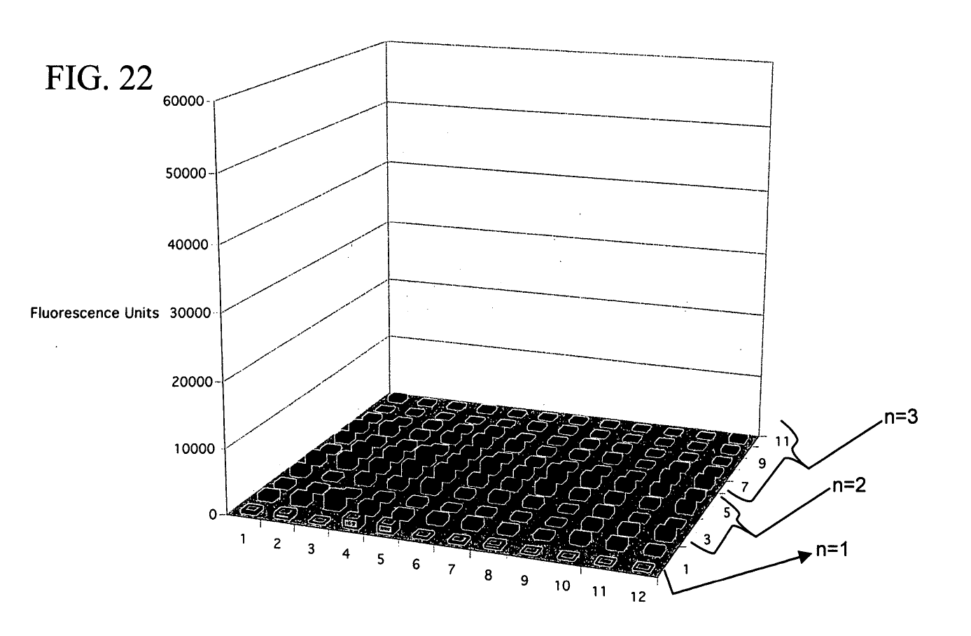

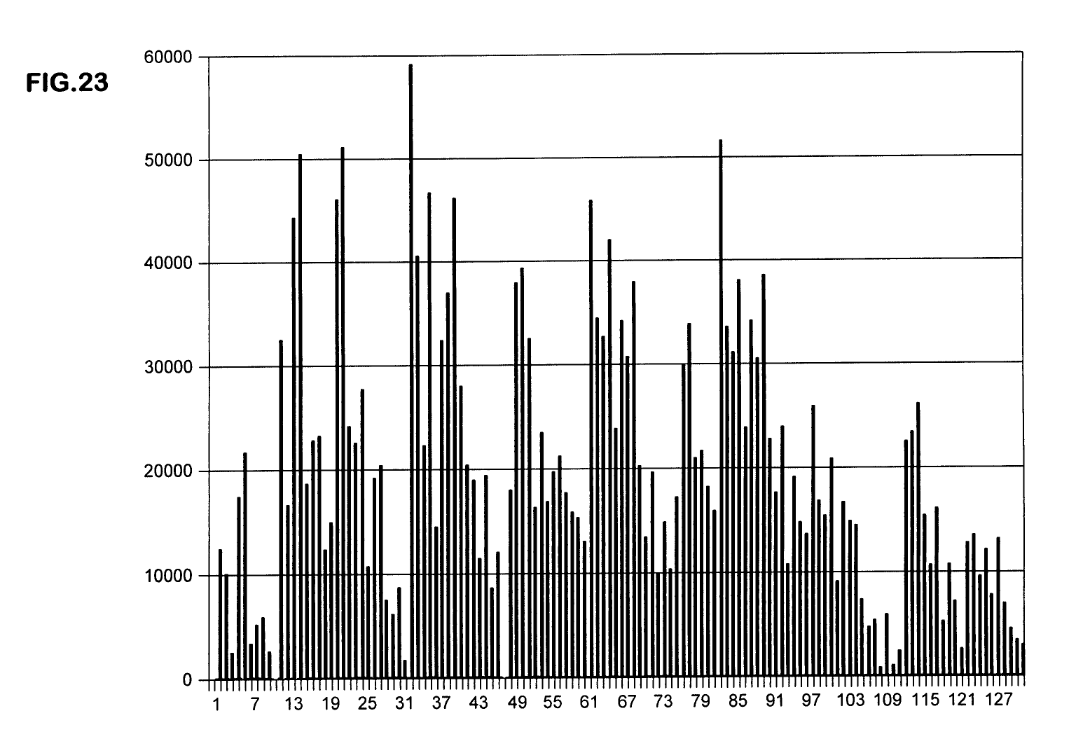

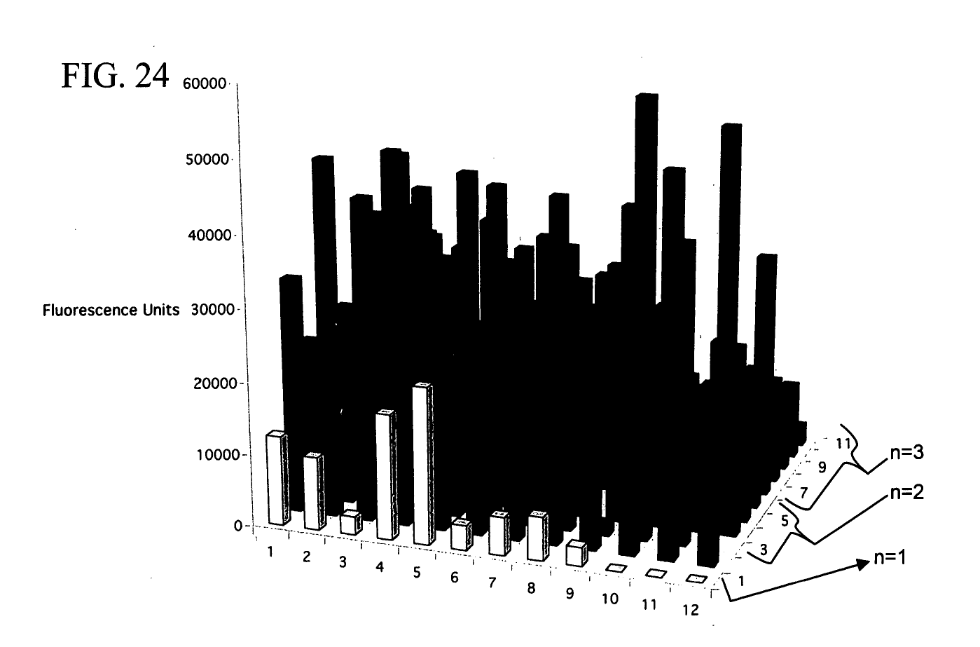

FIGS. 15 and 16 illustrate binding data for r-phycoerythrin (intrinsic fluorescence). FIGS. 17 and 18 illustrate binding data for ovalbumin (commercially available with fluorescence label). FIGS. 19 and 20 illustrate binding data for bovine serum albumin (labeled with rhodamine). FIGS. 21 and 22 illustrate binding data for HRP-NH-Ac (fluorescent tyramide read-out). FIGS. 23 and 24 illustrate binding data for HRP-NH-TCDD (fluorescent tyramide read-out).

These results demonstrate not only the application of the CARA microarray to candidate artificial receptor evaluation but also a few of the many read-out methods (e.g. intrinsic fluorescence, fluorescently labeled, in situ fluorescence labeling) which can be utilized for high throughput candidate receptor evaluation.

The evaluation of candidate receptors benefits from reproducibility. The following results demonstrate that the present microarrays provided reproducible ligand binding.

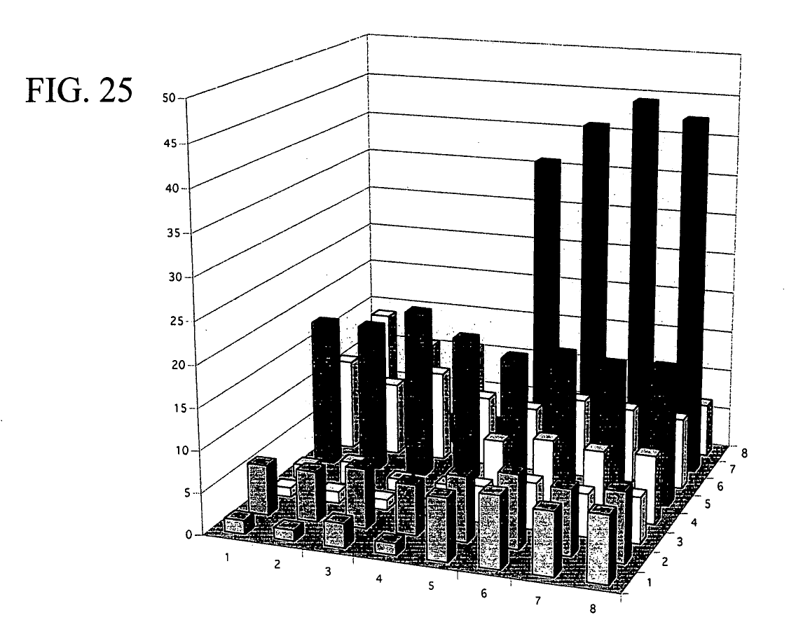

The microarrays were printed with each combination of building blocks spotted in quadruplicate. Visual inspection of a direct plot (FIG. 25) of the raw fluorescence data (from the run illustrated in FIG. 14) for one block of binding data obtained for r-phycoerythrin demonstrates that the candidate receptor environment “spots” showed reproducible binding to the test ligand. Further analysis of the r-phycoerythrin data (FIG. 14) led to only 9 out of 768 spots (1.2%) being deleted as outliers. Analysis of the r-phycoerythrin quadruplicate data for the entire array gives a mean standard deviation for each experimental quadruplicate set of 938 fluorescence units, with a mean coefficient of variation of 19.8%.

Although these values are acceptable, a more realistic comparison employed the standard deviation and coefficient of variation of the more strongly bound, more fluorescent receptors. The overall mean standard deviation unrealistically inflates the coefficient of variation for the weakly bound, less fluorescent receptors. The coefficient of variation for the 19 receptors with greater than 10,000 Fluorescent Units of bound target is 11.1%, which is well within the range required to produce meaningful binding data.

One goal of the CARA approach is the facile preparation of a significant number of candidate receptors through combinations of structurally simple building blocks. The following results establish that both the individual building blocks and combinations of building blocks have a significant, positive effect on test ligand binding.

The binding data illustrated in FIGS. 23-24 demonstrate that heterogeneous combinations of building blocks (n=2, n=3) are dramatically superior candidate receptors made from a single building block (n=1). For example, FIG. 16 illustrates both the diversity of binding observed for n=2, n=3 candidate receptors with fluorescent units ranging from 0 to ca. 40,000. These data also illustrate and the ca. 10-fold improvement in binding affinity obtained upon going from the homogeneous (n=1) to heterogeneous (n=2, n=3) receptor environments.

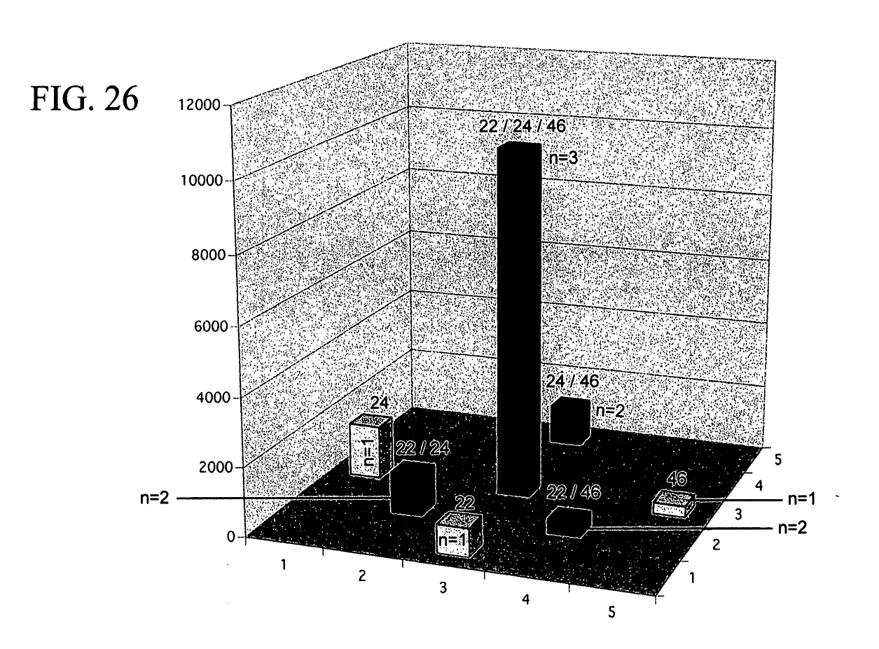

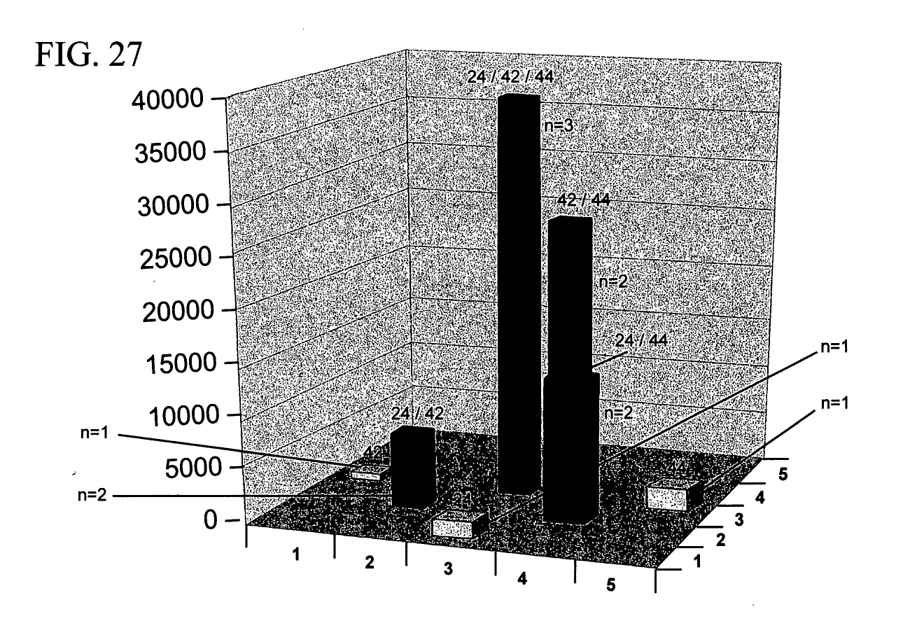

The effect of heterogeneous building blocks is most easily observed by comparing selected n=3 receptor environments candidate receptors including 1 or 2 of those building blocks (their n=2 and n=1 subsets). FIGS. 26 and 27 illustrate this comparison for two different n=3 receptor environments using the r-phycoerythrin data. In these examples, it is clear that progression from the homogeneous system (n=1) to the heterogeneous systems (n=2, n=3) produces significantly enhanced binding.

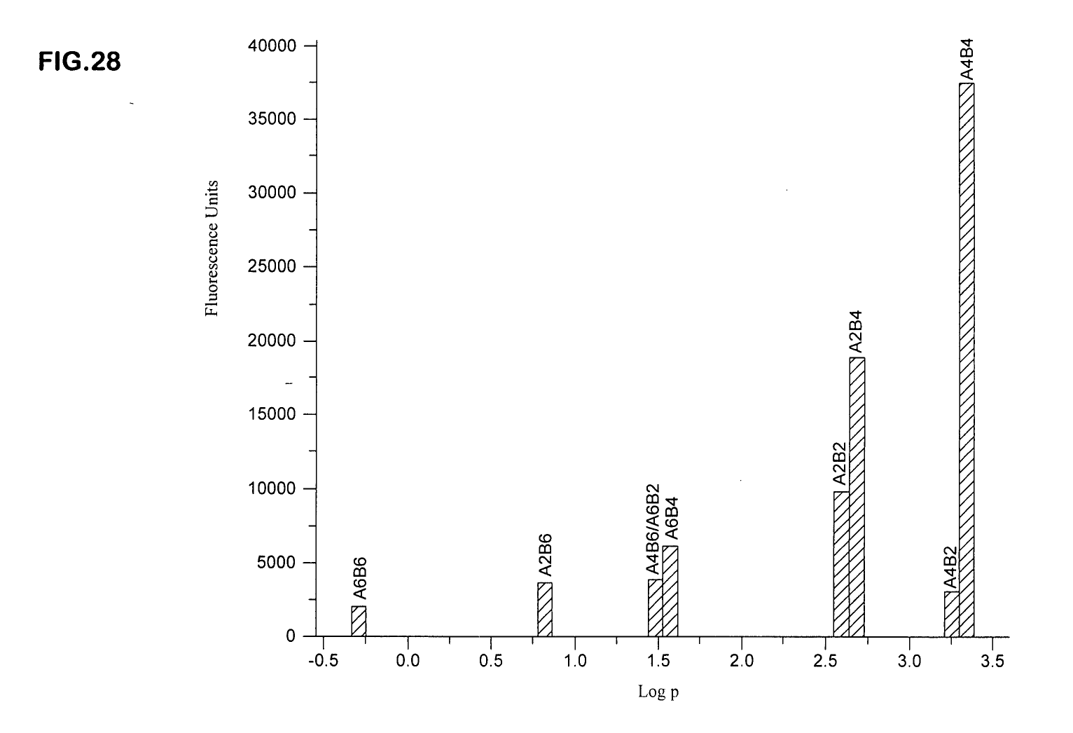

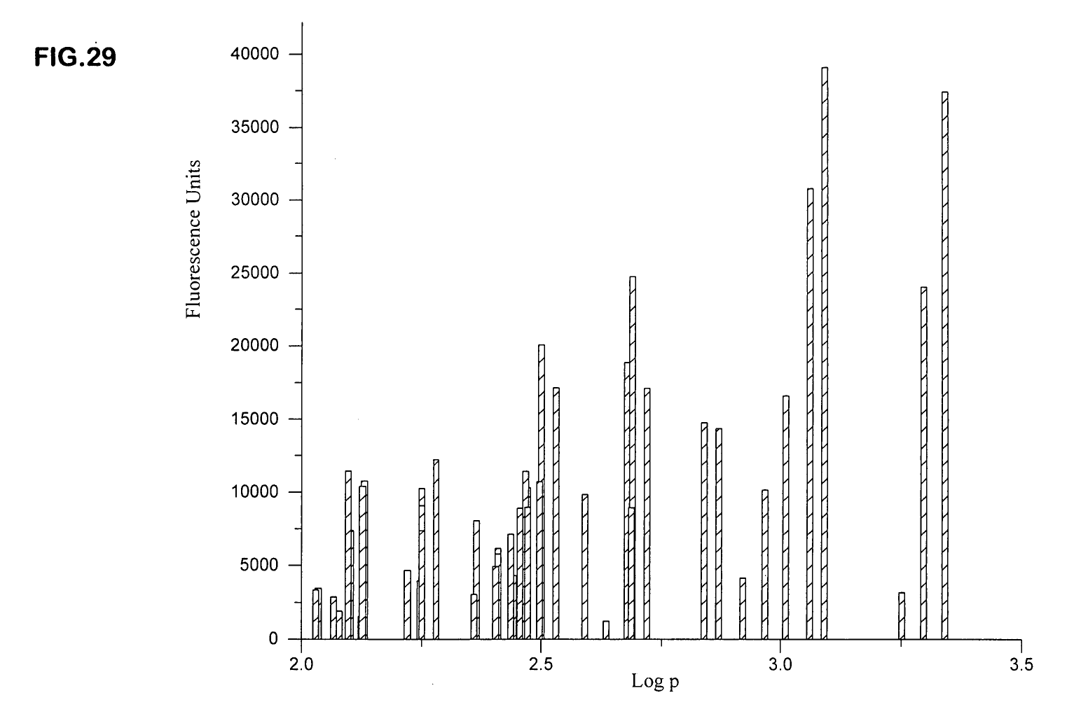

Although van der Waals interactions are an important part of molecular recognition, it is important to establish that the observed binding is not a simple case of hydrophobic/hydrophilic partitioning. That is, that the observed binding was the result of specific interactions between the individual building blocks and the target The simplest way to evaluate the effects of hydrophobicity and hydrophilicity is to compare building block logP value with observed binding. LogP is a known and accepted measure of lipophilicity, which can be measured or calculated by known methods for each of the building blocks. FIGS. 28 and 29 establish that the observed target binding, as measured by fluorescence units, is not directly proportional to building block logP. The plots in FIGS. 28 and 29 illustrate a non-linear relationship between binding (fluorescence units) and building block logP.

One advantage of the present methods and arrays is that the ability to screen large numbers of candidate receptor environments will lead to a combination of useful target affinities and to significant target binding diversity. High target affinity is useful for specific target binding, isolation, etc. while binding diversity can provide multiplexed target detection systems. This example employed a relatively small number of building blocks to produce ca. 120 binding environments. The following analysis of the present data clearly demonstrates that even a relatively small number of binding environments can produce diverse and useful artificial receptors.

The target binding experiments performed for this study used protein concentrations including 0.1 to 10 μg/ml. Considering the BSA data as representative, it is clear that some of the receptor environments readily bound 1.0 ug/ml BSA concentrations near the saturation values for fluorescence units (see, e.g., FIG. 20). Based on these data and the formula weight of 68,000 for BSA, several of the receptor environments readily bind BSA at ca. 15 picomole/ml or 15 nanomolar concentrations. Additional experiments using lower concentrations of protein (data not shown) indicate that, even with a small selection of candidate receptor environments, femptomole/ml or picomolar detection limits have been attained.

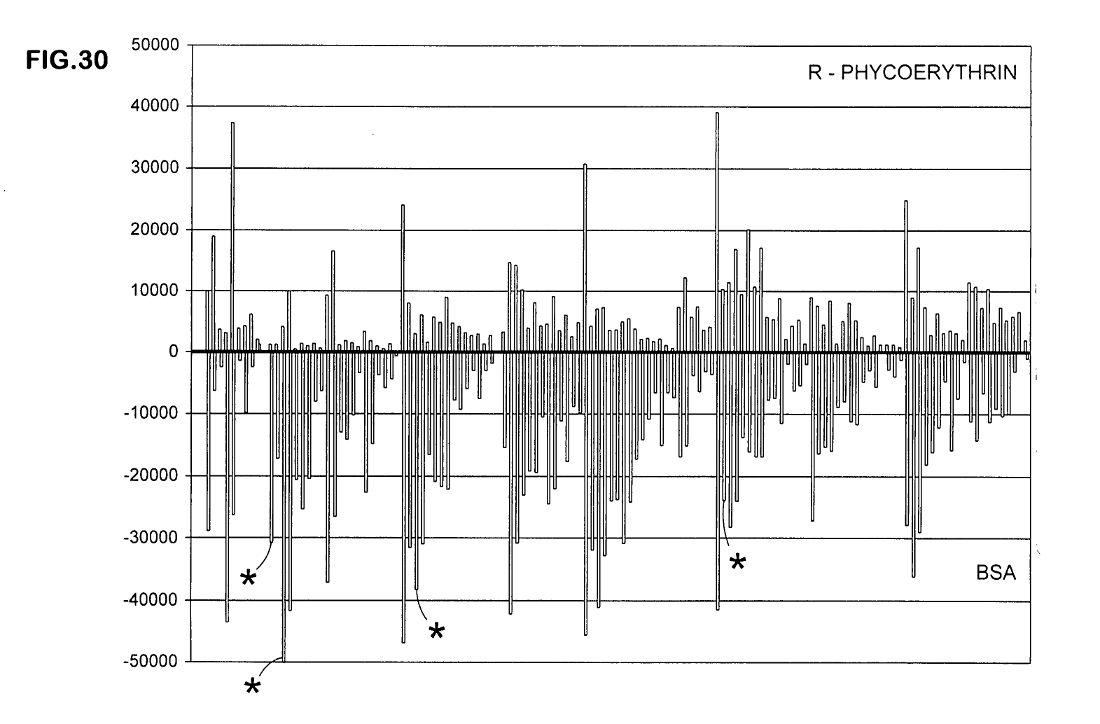

One goal of artificial receptor development is the specific recognition of a particular target. FIG. 30 compares the observed binding for r-phycoerythrin and BSA. Comparison of the overall binding pattern indicates some general similarities. However, comparison of specific features of binding for each receptor environment demonstrates that the two targets have distinctive recognition features as indicated by the (*) in FIG. 30.

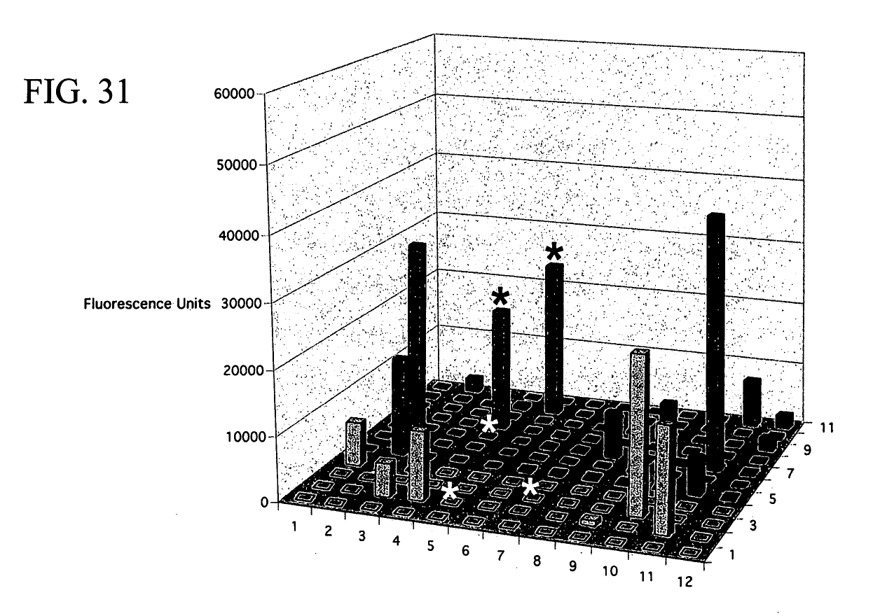

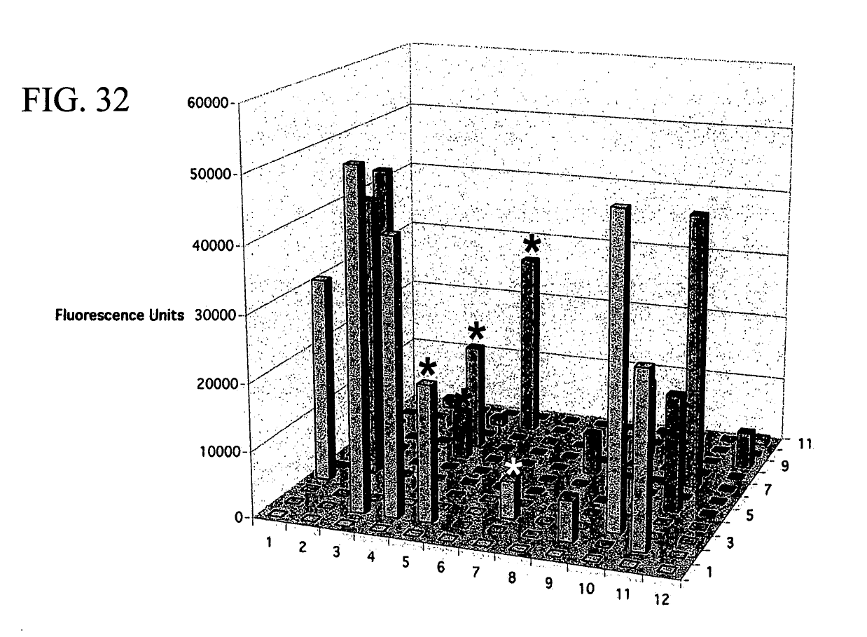

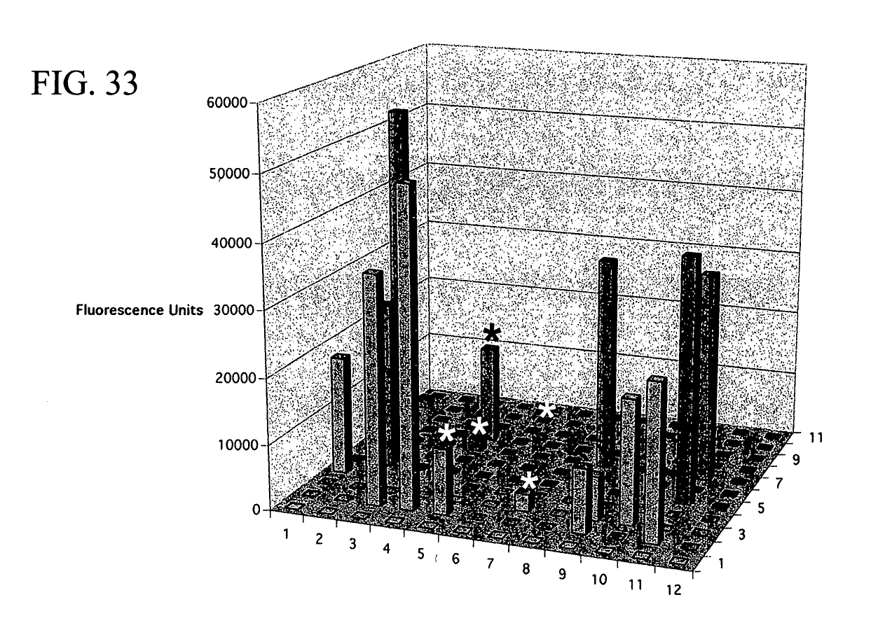

One goal of artificial receptor development is to develop receptors which can be used for the multiplexed detection of specific targets. Comparison of the r-phycoerythrin, BSA and ovalbumin data from this study (FIGS. 16, 18, and 20) were used to select representative artificial receptors for each target. FIGS. 31, 32 and 33 employ data obtained in the present example to illustrate identification of each of these three targets by their distinctive binding patterns.

Conclusions

The optimum receptor for a particular target requires molecular recognition which is greater than the expected sum of the individual hydrophilic, hydrophobic, ionic, etc. interactions. Thus, the identification of an optimum (specific, sensitive) artificial receptor from the limited pool of candidate receptors explored in this prototype study, was not expected and not likely. Rather, the goal was to demonstrate that all of the key components of the CARA: Combinatorial Artificial Receptor Array concept could be assembled to form a functional receptor microarray. This goal has been successfully demonstrated.

This study has conclusively established that CARA microarrays can be readily prepared and that target binding to the candidate receptor environments can be used to identify artificial receptors and test ligands. In addition, these results demonstrate that there is significant binding enhancement for the building block heterogeneous (n=2, n=3, or n=4) candidate receptors when compared to their homogeneous (n=1) counterparts. When combined with the binding pattern recognition results and the demonstrated importance of both the heterogeneous receptor elements and heterogeneous building blocks, these results clearly demonstrate the significance of the CARA Candidate Artificial Receptor->Lead Artificial Receptor->Working Artificial Receptor strategy.

### Example 3

### Preparation and Evaluation of Microarrays of Candidate Artificial Receptors Including Reversibly Immobilized Building Blocks

Microarrays of candidate artificial receptors including building blocks immobilized through van der Waals interactions were made and evaluated for binding of a protein ligand. The evaluation was conducted at several temperatures, above and below a phase transition temperature for the lawn (vide infra).

Materials and Methods

Building blocks 2-2, 2-4, 2-6, 4-2, 4-4, 4-6, 6-2, 6-4, 6-6 where prepared as described in Example 1. The C12 amide was prepared using the previously described carbodiimide activation of the carboxyl followed by addition of dodecylamine. This produced a building block with a 12 carbon alkyl chain linker for reversible immobilization in the C18 lawn.

Amino lawn microarray plates (Telechem) were modified to produce the C18 lawn by reaction of stearoyl chloride (Aldrich Chemical Co.) in A) dimethylformamide/PEG 400 solution (90:10, v/v, PEG 400 is polyethylene glycol average MW 400 (Aldrich Chemical Co.) or B) methylene chloride/TEA solution (100 ml methylene chloride, 200 μl triethylamine) using the lawn modification procedures generally described in Example 2.

The C18 lawn plates where printed using the SpotBot standard procedure as described in Example 2. The building blocks were in printing solutions prepared by solution of ca. 10 mg of each building block in 300 μl of methylene chloride and 100 μl methanol. To this stock was added 900 μl of dimethylformamide and 100 μl of PEG 400. The 36 combinations of the 9 building blocks taken two at a time (N9:n2, 36 combinations) where prepared in a 384-well microwell plate which was then used in the SpotBot to print the microarray in quadruplicate. A random selection of the print positions contained only print solution.

The selected microarray was incubated with a 1.0 μg/ml solution of the test ligand, cholera toxin subunit B labeled with the Alexa™ fluorophore (Molecular Probes Inc., Eugene, Oreg.), using the following variables: 1) the microarray was washed with methylene chloride, ethanol and water to create a control plate; and 2) the microarray was incubated at 4° C., 23° C., or 44° C. After incubation, the plate(s) were rinsed with water, dried and scanned (AXON 4100A). Data analysis was as described in Example 2.

Results

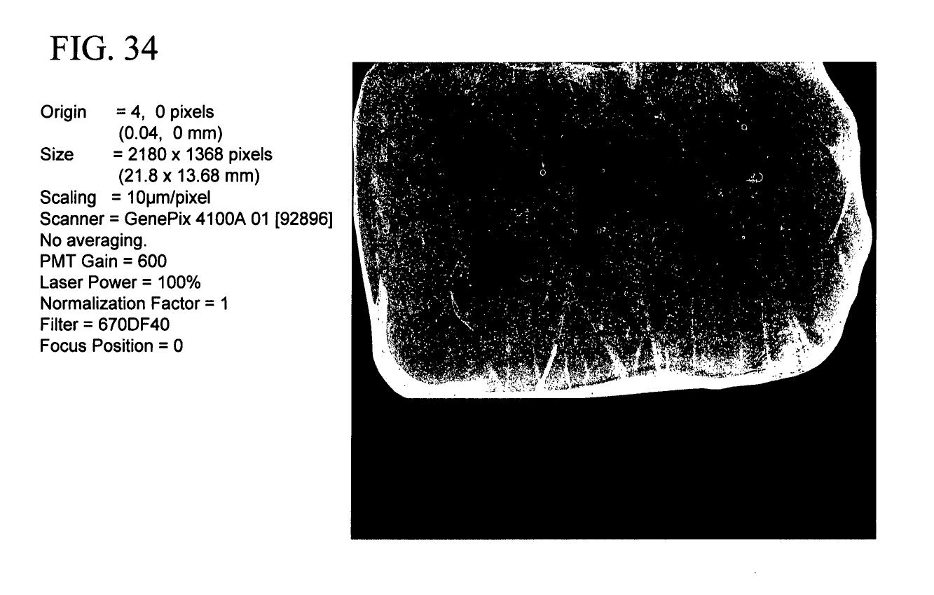

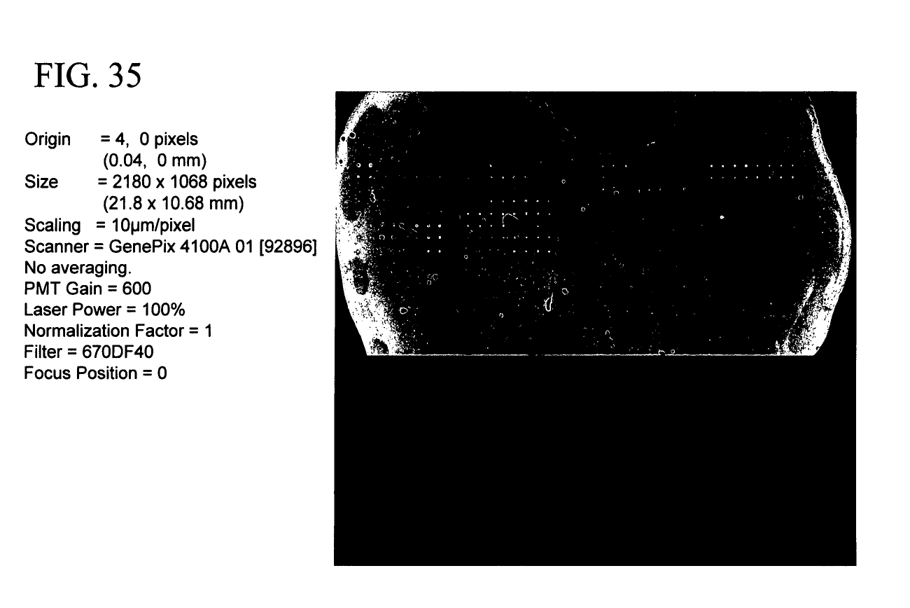

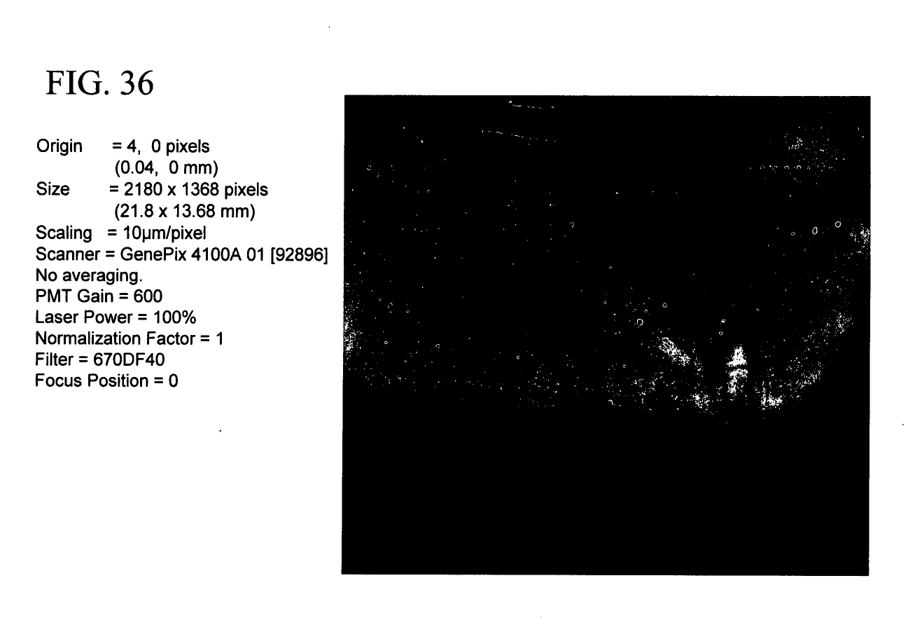

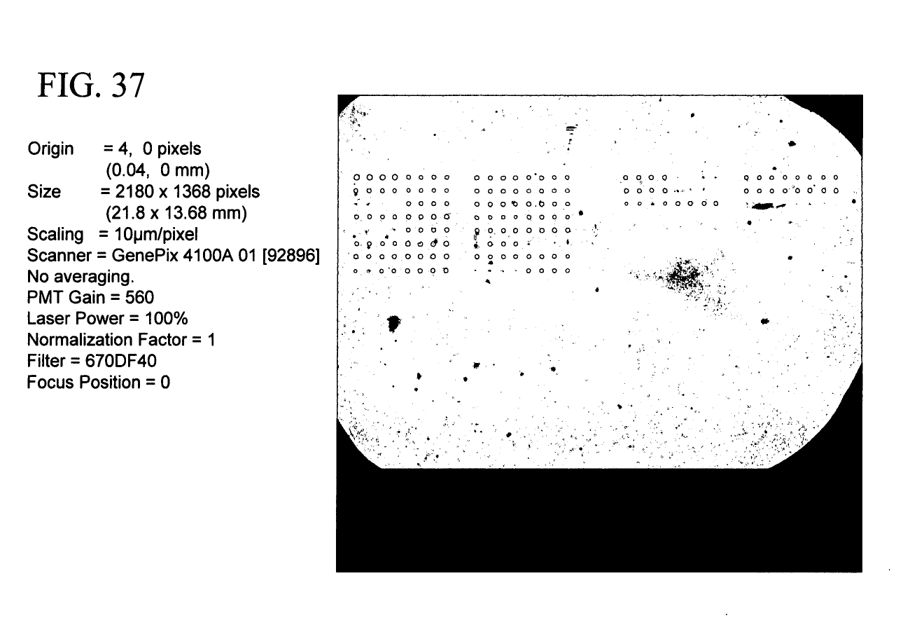

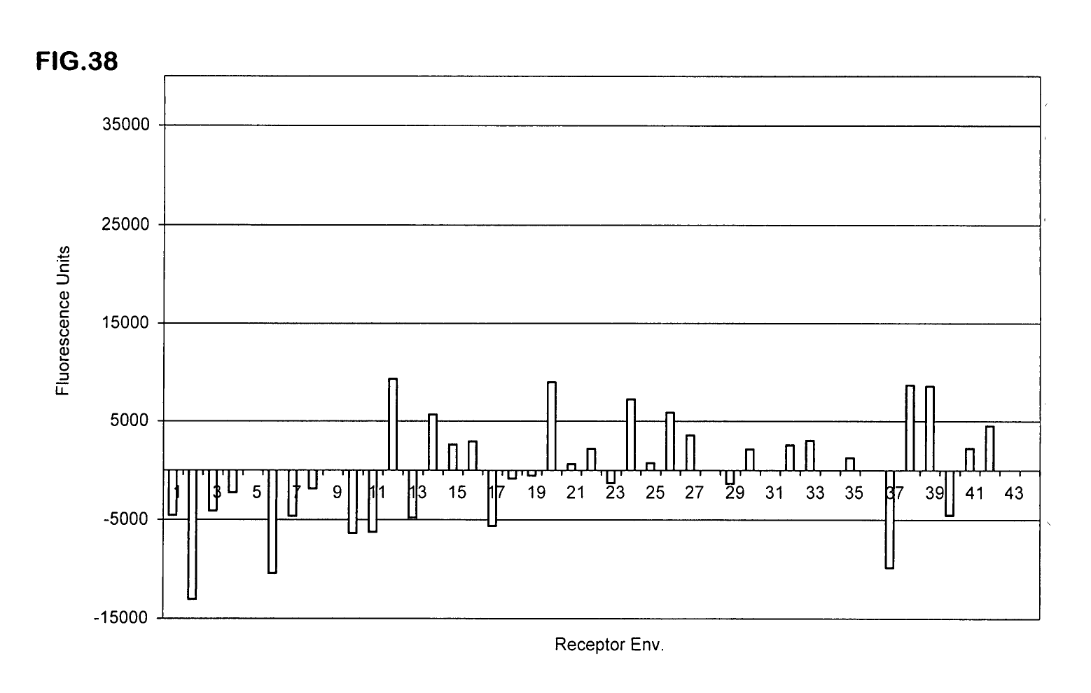

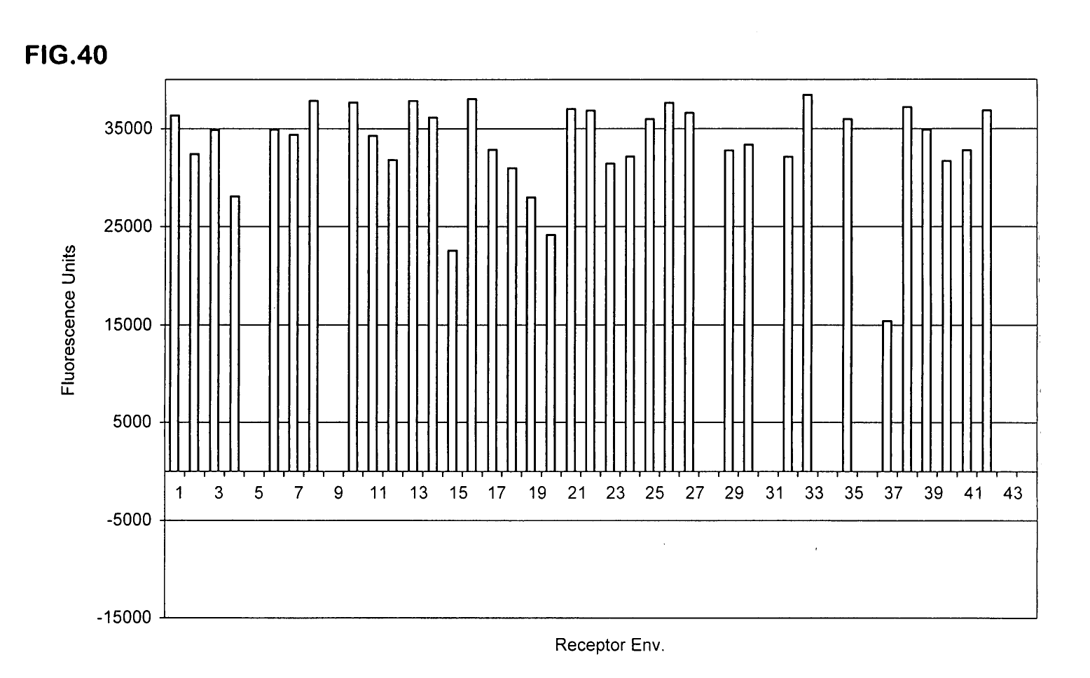

A control array from which the building blocks had been removed by washing with organic solvent did not bind cholera toxin (FIG. 34). FIGS. 35-37 illustrate fluorescence signals from arrays printed identically, but incubated with cholera toxin at 4° C., 23° C., or 44° C., respectively. Spots of fluorescence can be seen in each array, with very pronounced spots produced by incubation at 44° C. The fluorescence values for the spots in each of these three arrays are shown in FIGS. 38-40. Fluorescence signal generally increases with temperature, with many nearly equally large signals observed after incubation at 44° C. Linear increases with temperature can reflect expected improvements in binding with temperature. Nonlinear increases reflect rearrangement of the building blocks on the surface to achieve improved binding, which occurred above the phase transition for the lipid surface (vide infra).

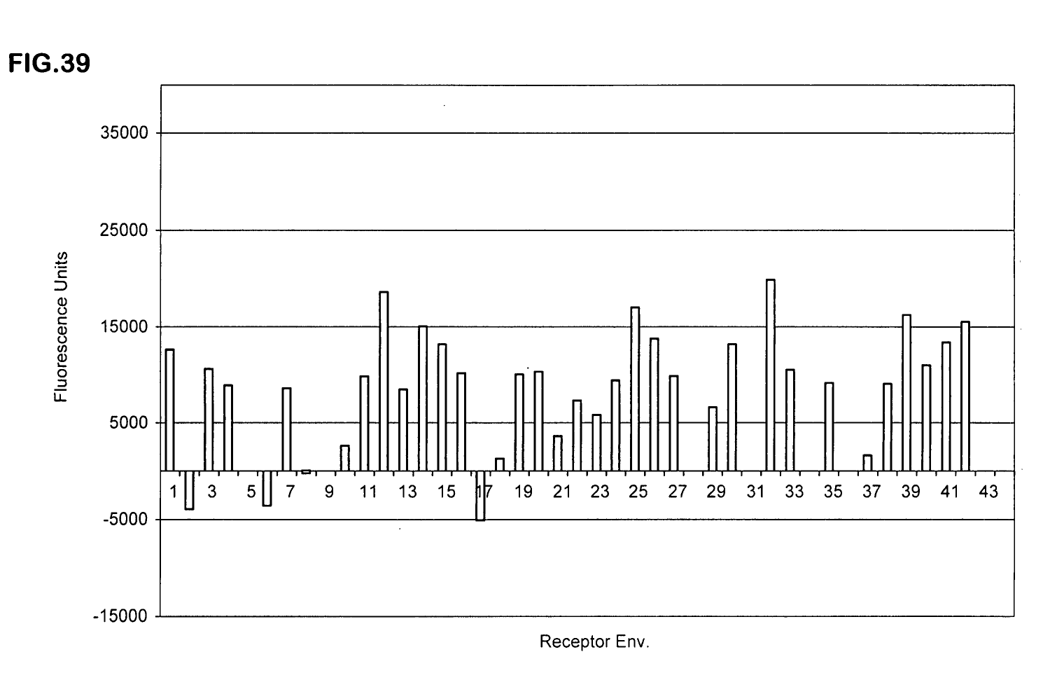

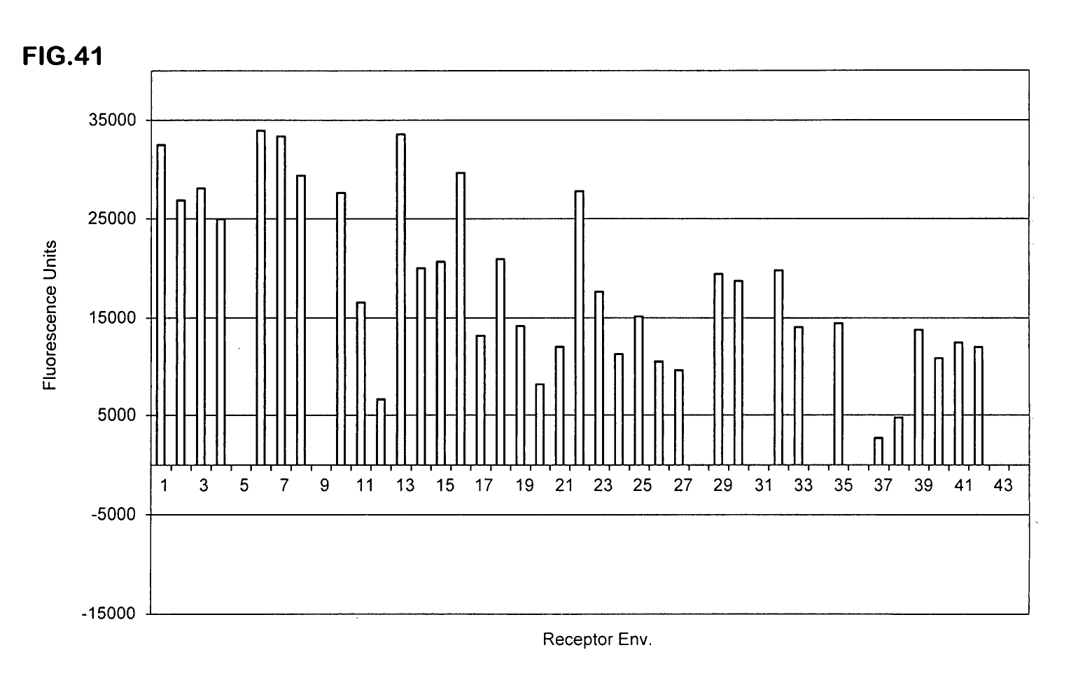

FIG. 41 can be compared to FIG. 39. The fluorescence signals plotted in FIG. 39 resulted from binding to reversibly immobilized building blocks on a support at 23° C. The fluorescence signals plotted in FIG. 41 resulted from binding to covalently immobilized building blocks on a support at 23° C. These figures compare the same combinations of building blocks in the same relative positions, but immobilized in two different ways.

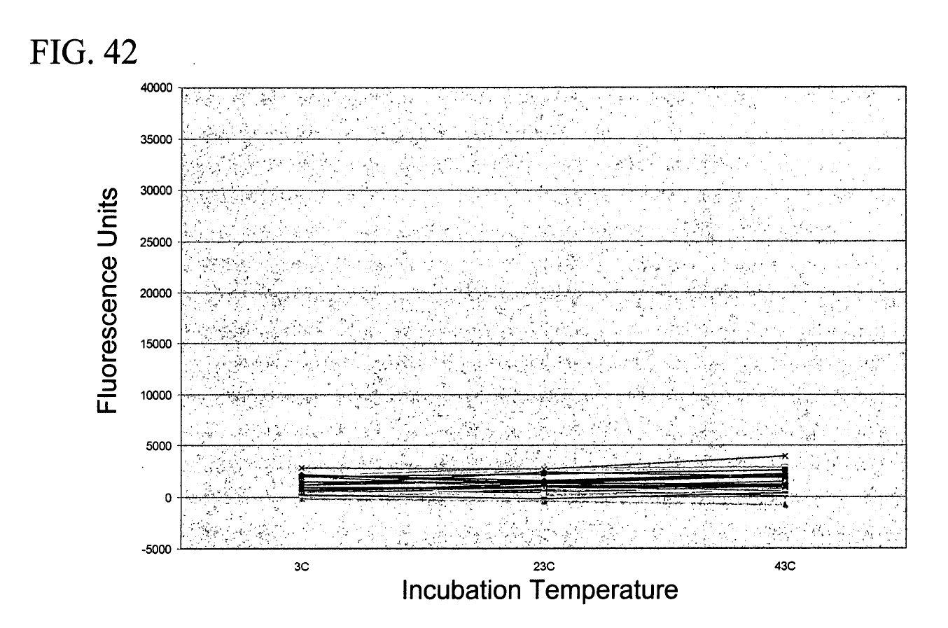

The binding to covalently immobilized building blocks was also evaluated at 4° C., 23° C., or 44° C. FIG. 42 illustrates the changes in fluorescence signal from individual combinations of covalently immobilized building blocks at 4° C., 23° C., or 44° C. Binding increased modestly with temperature. The mean increase in binding was 1.3-fold. A plot of the fluorescence signal for each of the covalently immobilized artificial receptors at 23° C. against its signal at 44° C. (not shown) yields a linear correlation with a correlation coefficient of 0.75. This linear correlation indicates that the mean 1.3-fold increase in binding is a thermodynamic effect and not optimization of binding.

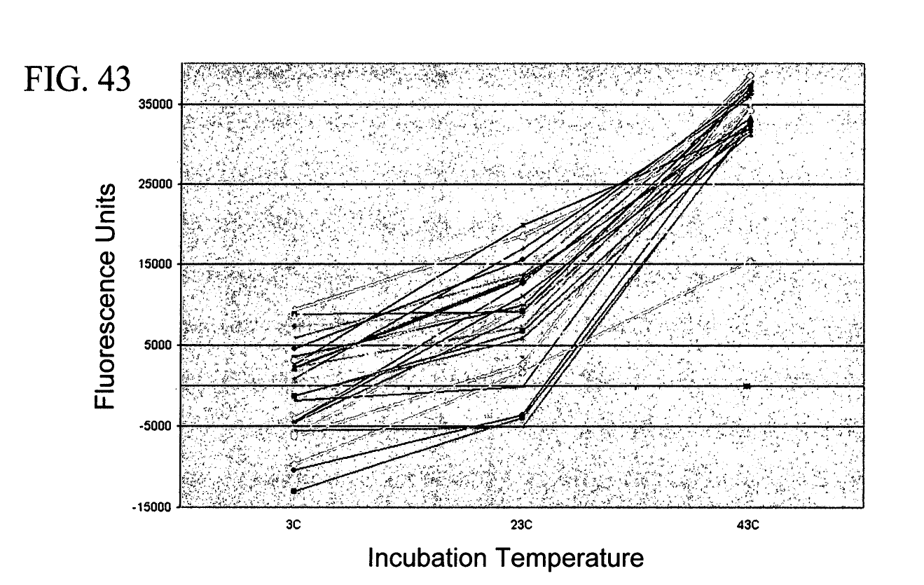

FIG. 43 illustrates the changes in fluorescence signal from individual combinations of reversibly immobilized building blocks at 4° C., 23° C., or 44° C. This graph illustrates that at least one combination of building blocks (candidate artificial receptor) exhibited a signal that remained constant as temperature increased. At least one candidate artificial receptor exhibited an approximately linear increase in signal as temperature increased. Such a linear increase indicates normal temperature effects on binding. The candidate artificial receptor with the lowest binding signal at 4° C. became one of the best binders at 44° C. This indicates that rearrangement of the building blocks of this receptor above the phase transition for the lawn, which increases the building blocks' mobility, produced increased binding. Other receptors characterized by greater changes in binding between 23° C. and 44° C. (compared to between 4° C. and 23° C.) also underwent dynamic affinity optimization.

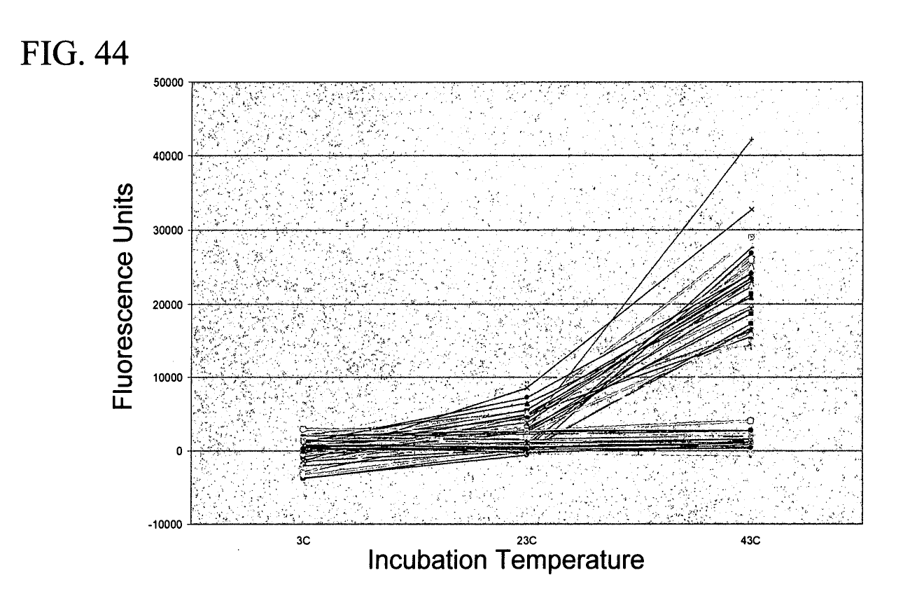

FIG. 44 illustrates the data presented in FIG. 42 (lines marked A) and the data presented in FIG. 43 (lines marked B). The increases in binding observed with the reversibly immobilized building blocks are significantly greater than the increases observed with covalently bound building blocks. Binding to reversibly immobilized building blocks increased from 23° C. and 44° C. by a median value of 6.1-fold and a mean value of 24-fold. This confirms that movement of the reversibly immobilized building blocks within the receptors increased binding (i.e., the receptor underwent dynamic affinity optimization).

A plot of the fluorescence signal for each of the reversibly immobilized artificial receptors at 23° C. against its signal at 44° C. (not shown) yields no correlation (correlation coefficient of 0.004). A plot of the fluorescence signal for each of the reversibly immobilized artificial receptors at 44° C. against the signal for the corresponding covalently immobilized receptor (not shown) also yields no correlation (correlation coefficient 0.004). This lack of correlation provides further evidence that movement of the reversibly immobilized building blocks within the receptors increased binding.

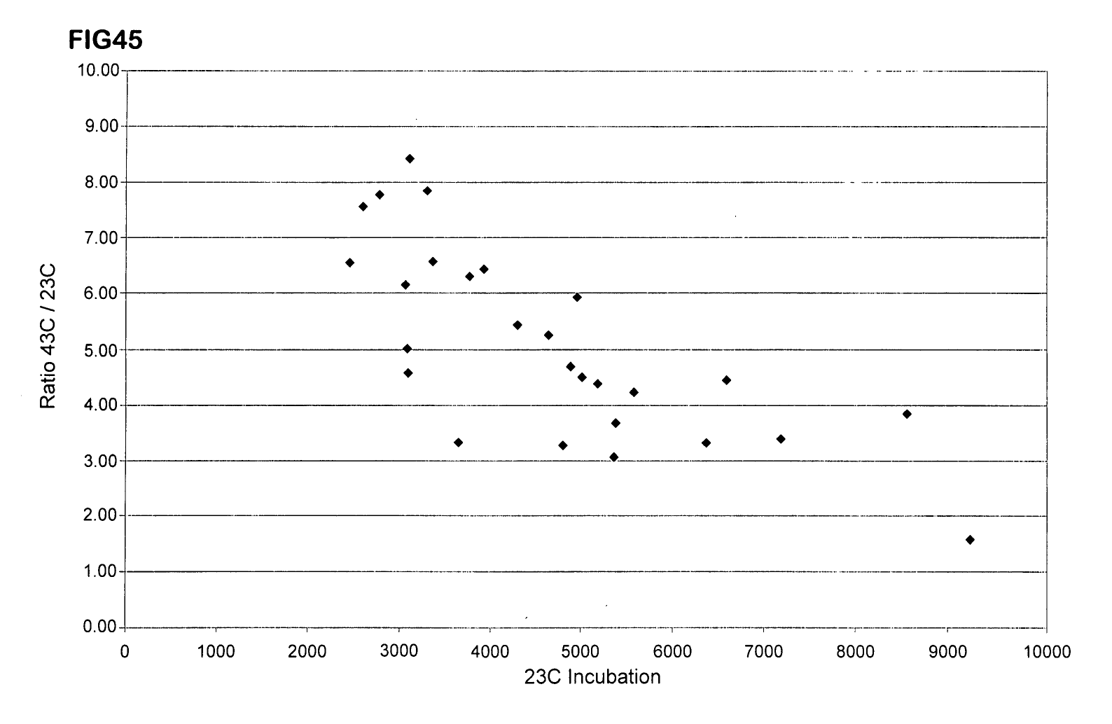

FIG. 45 illustrates a graph of the fluorescence signal at 44° C. divided by the signal at 23° C. against the fluorescence signal obtained from binding at 23° C. for the artificial receptors with reversibly immobilized receptors. This comparison indicates that the binding enhancement is independent of the initial affinity of the receptor for the test ligand.

Table 1 identifies the reversibly immobilized building blocks making up each of the artificial receptors, lists the fluorescence signal (binding strength) at 44° C. and 23° C., and the ratios of the observed binding at these two temperatures. These data illustrate that each artificial receptor reflects a unique attribute for each combination of building blocks relative to the role of each individual building block.

TABLE 1

| Building Blocks | | | Ratio of |
|---|---|---|---|
| Making Up | | | Signals, |
| Receptor | Signal at 44° C. | Signal at 23° C. | 44° C./23° C. |
| 22 24 | 24136 | 4611 | 5.23 |
| 22 26 | 16660 | 43 | 387.44 |
| 22 42 | 17287 | −167 | −103.51 |
| 22 44 | 16726 | 275 | 60.82 |
| 22 46 | 25016 | 3903 | 6.41 |
| 22 62 | 13990 | 3068 | 4.56 |
| 22 64 | 15294 | 3062 | 4.99 |
| 22 66 | 11980 | 3627 | 3.30 |
| 24 26 | 22688 | 1291 | 17.57 |
| 24 42 | 26808 | −662 | −40.50 |
| 24 44 | 23154 | 904 | 25.61 |
| 24 46 | 42197 | 2814 | 15.00 |
| 24 62 | 19374 | 2567 | 7.55 |
| 24 64 | 27599 | 262 | 105.34 |
| 24 66 | 16238 | 5334 | 3.04 |
| 26 42 | 22282 | 4974 | 4.48 |
| 26 44 | 26240 | 530 | 49.51 |
| 26 46 | 23144 | 4273 | 5.42 |
| 26 62 | 29022 | 4920 | 5.90 |
| 26 64 | 23416 | 5551 | 4.22 |
| 26 66 | 19553 | 5353 | 3.65 |
| 42 44 | 29093 | 6555 | 4.44 |
| 42 46 | 18637 | 3039 | 6.13 |
| 42 62 | 22643 | 4853 | 4.67 |
| 42 64 | 20836 | 6343 | 3.28 |
| 42 66 | 14391 | 9220 | 1.56 |
| 44 46 | 25600 | 3266 | 7.84 |
| 44 62 | 15544 | 4771 | 3.26 |
| 44 64 | 25842 | 3073 | 8.41 |
| 44 66 | 22471 | 5142 | 4.37 |
| 46 62 | 32764 | 8522 | 3.84 |
| 46 64 | 21901 | 3343 | 6.55 |
| 46 66 | 23516 | 3742 | 6.28 |
| 62 64 | 24069 | 7149 | 3.37 |
| 62 66 | 15831 | 2424 | 6.53 |
| 64 66 | 21310 | 2746 | 7.76 |

Conclusions

This experiment demonstrated that an array including reversibly immobilized building blocks binds a protein substrate, like an array with covalently immobilized building blocks. The binding increased nonlinearly as temperature increased, indicating that movement of the building blocks increased binding. Many of the candidate artificial receptors demonstrated improved binding upon mobilization of the building blocks.

### Example 4

### The Oligosaccharide Portion of GM1 Competes with Artificial Receptors for Binding to Cholera Toxin

Microarrays of candidate artificial receptors were made and evaluated for binding of cholera toxin. The arrays were also evaluated for disrupting that binding. Disrupting of binding employed a compound that binds to cholera toxin, the oligosaccharide moiety from GM1 (GM1 OS). The results obtained demonstrate that a ligand of a protein specifically disrupted binding of the protein to the microarray.

Materials and Methods

Building blocks were synthesized and activated as described in Example 1. The building blocks employed in this example were TyrA1B1 [1-1], TyrA2B2, TyrA2B4, TyrA2B6, TyrA2B8, TyrA3B3, TyrA3B5, TyrA3B7, TyrA4B2, TyrA4B4, TyrA4B6, TyrA4B8, TyrA5B3, TyrA5B5, TyrA5B7, TyrA6B2, TyrA6B4, TyrA6B6, TyrA6B8, TyrA7B3, TyrA7B5, TyrA7B7, TyrA8B2, TyrA8B4, TyrA8B6, and TyrA8B8. The abbreviation for the building block including a linker, a tyrosine framework, and recognition elements AxBy is TyrAxBy.

Microarrays for the evaluation of the 171 n=2 candidate receptor environments were prepared as follows by modifications of known methods. An “n=2” receptor environment includes two different building blocks. Briefly: Amine modified (amine “lawn”; SuperAmine Microarray plates) microarray plates were purchased from Telechem Inc., Sunnyvale, Calif. These plates were manufactured specifically for microarray preparation and had a nominal amine load of 2-4 amines per square nm according to the manufacturer. The microarrays were prepared using a pin microarray spotter instrument from Telechem Inc. (SpotBot™ Arrayer) typically with 200 μm diameter spotting pins from Telechem Inc. (Stealth Micro Spotting Pins, SMP6) and 400-420 μm spot spacing.

The 19 building blocks were activated in aqueous dimethylformamide (DMF) solution as described above. For preparing the 384-well feed plate, the activated building block solutions were diluted 10-fold with a solution of DMF/H₂O/PEG400 (90/10/10, v/v/v; PEG400 is polyethylene glycol nominal 400 FW, Aldrich Chemical Co., Milwaukee, Wis.). These stock solutions were aliquotted (10 μl per aliquot) into the wells of a 384-well microwell plate (Telechem Inc.). Control spots included the building block [1-1]. The plate was covered with aluminum foil and placed on the bed of a rotary shaker for 15 minutes at 1,000 RPM. This master plate was stored covered with aluminum foil at −20° C. when not in use.

For preparing the 384-well SpotBot™ plate, a well-to-well transfer (e.g. A-1 to A-1, A-2 to A-2, etc.) from the feed plate to a second 384-well plate was performed using a 4 μl transfer pipette. This plate was stored tightly covered with aluminum foil at −20° C. when not in use. The SpotBot™ was used to prepare up to 13 microarray plates per run using the 4 μl microwell plate. The SpotBot™ was programmed to spot from each microwell in quadruplicate. The wash station on the SpotBot™ used a wash solution of EtOH/H₂O (20/80, v/v). This wash solution was adjusted to pH 4 with 1 M HCl and used to rinse the microarrays on completion of the SpotBot™ printing run. The plates were given a final rinse with deionized (DI) water, dried using a stream of compressed air, and stored at room temperature. The microarrays were further modified by reacting the remaining amines with acetic anhydride to form an acetamide lawn in place of the amine lawn.

The test ligand employed in these experiments was cholera toxin labeled with the Alexa™ fluorophore (Molecular Probes Inc., Eugene, Oreg.). The candidate disruptor employed in these experiments was GM1 OS (GM1 oligosaccharide), a known ligand for cholera toxin.

Microarray incubation and analysis was conducted as follows: For control incubations with the microarrays, solutions (e.g. 500 μl) of the cholera toxin in PBS-T (PBS with 20 μl/L of Tween-20) at a concentrations of 1.7 pmol/ml (0.1 μg/ml) was placed onto the surface of a microarray and allowed to react for 30 minutes. For disruptor incubations with the microarrays, solutions (e.g. 500 μl) of the cholera toxin (1.7 pmol/ml, 0.1 μg/ml) and the desired concentration of GM1 OS in PBS-T (PBS with 20 μl/L of Tween-20) was placed onto the surface of a microarray and allowed to react for 30 minutes. GM1 OS was added at 0.34 and at 5.1 μM in separate experiments. After either of these incubations, the microarray was rinsed with PBS-T and DI water and dried using a stream of compressed air.

The incubated microarray was scanned using an Axon Model 4200A Fluorescence Microarray Scanner (Axon Instruments, Union City, Calif.). The Axon scanner and its associated software produce a false color 16-bit image of the fluorescence intensity of the plate. This 16-bit data is integrated using the Axon software to give a Fluorescence Units value (range 0-65,536) for each spot on the microarray. This data is then exported into an Excel file (Microsoft) for further analysis including mean, standard deviation and coefficient of variation calculations.

Table 2 identifies the building blocks in each of the 171 receptor environments.

TABLE 2

| | Building |
|---|---|
| | Blocks |
| 1 | 22 24 |
| 2 | 22 26 |
| 3 | 22 28 |
| 4 | 22 33 |
| 5 | 22 42 |
| 6 | 22 44 |
| 7 | 22 46 |
| 8 | 22 48 |
| 9 | 22 55 |
| 10 | 22 62 |
| 11 | 22 64 |
| 12 | 22 66 |
| 13 | 22 68 |
| 14 | 22 77 |
| 15 | 22 82 |
| 16 | 22 84 |
| 17 | 22 86 |
| 18 | 22 88 |
| 19 | 24 26 |
| 20 | 24 28 |
| 21 | 24 33 |
| 22 | 24 42 |
| 23 | 24 44 |
| 24 | 24 46 |
| 25 | 24 48 |
| 26 | 24 55 |
| 27 | 24 62 |
| 28 | 24 64 |
| 29 | 24 66 |
| 30 | 24 68 |
| 31 | 24 77 |
| 32 | 24 82 |
| 33 | 24 84 |
| 34 | 24 86 |
| 35 | 24 88 |
| 36 | 26 28 |
| 37 | 26 33 |
| 38 | 26 42 |
| 39 | 26 44 |
| 40 | 26 46 |
| 41 | 26 48 |
| 42 | 26 55 |
| 43 | 26 62 |
| 44 | 26 64 |
| 45 | 26 66 |
| 46 | 26 68 |
| 47 | 26 77 |
| 48 | 26 82 |
| 49 | 26 84 |
| 50 | 26 86 |
| 51 | 26 88 |
| 52 | 28 33 |
| 53 | 28 42 |
| 54 | 28 44 |
| 55 | 28 46 |
| 56 | 28 48 |
| 57 | 28 55 |
| 58 | 28 62 |
| 59 | 28 64 |
| 60 | 28 66 |
| 61 | 28 68 |
| 62 | 28 77 |
| 63 | 28 82 |
| 64 | 28 84 |
| 65 | 28 86 |
| 66 | 28 88 |
| 67 | 33 42 |
| 68 | 33 44 |
| 69 | 33 46 |
| 70 | 33 48 |
| 71 | 33 55 |
| 72 | 33 62 |
| 73 | 33 64 |
| 74 | 33 66 |
| 75 | 33 68 |
| 76 | 33 77 |
| 77 | 33 82 |
| 78 | 33 84 |
| 79 | 33 86 |
| 80 | 33 88 |
| 81 | 42 44 |
| 82 | 42 46 |
| 83 | 42 48 |
| 84 | 42 55 |
| 85 | 42 62 |
| 86 | 42 64 |
| 87 | 42 66 |
| 88 | 42 68 |
| 89 | 42 77 |
| 90 | 42 82 |
| 91 | 42 84 |
| 92 | 42 86 |
| 93 | 42 88 |
| 94 | 44 46 |
| 95 | 44 48 |
| 96 | 44 55 |
| 97 | 44 62 |
| 98 | 44 64 |
| 99 | 44 66 |
| 100 | 44 68 |
| 101 | 44 77 |
| 102 | 44 82 |
| 103 | 44 84 |
| 104 | 44 86 |
| 105 | 44 88 |
| 106 | 46 48 |
| 107 | 46 55 |
| 108 | 46 62 |
| 109 | 46 64 |
| 110 | 46 66 |
| 111 | 46 68 |
| 112 | 46 77 |
| 113 | 46 82 |
| 114 | 46 84 |
| 115 | 46 86 |
| 116 | 46 88 |
| 117 | 48 55 |
| 118 | 48 62 |
| 119 | 48 64 |
| 120 | 48 66 |
| 121 | 48 68 |
| 122 | 48 77 |
| 123 | 48 82 |
| 124 | 48 84 |
| 125 | 48 86 |
| 126 | 48 88 |
| 127 | 55 62 |
| 128 | 55 64 |
| 129 | 55 66 |
| 130 | 55 68 |
| 131 | 55 77 |
| 132 | 55 82 |
| 133 | 55 84 |
| 134 | 55 86 |
| 135 | 55 88 |
| 136 | 62 64 |
| 137 | 62 66 |
| 138 | 62 68 |
| 139 | 62 77 |
| 140 | 62 82 |
| 141 | 62 84 |
| 142 | 62 86 |
| 143 | 62 88 |
| 144 | 64 66 |
| 145 | 64 68 |
| 146 | 64 77 |
| 147 | 64 82 |
| 148 | 64 84 |
| 149 | 64 86 |
| 150 | 64 88 |
| 151 | 66 68 |
| 152 | 66 77 |
| 153 | 66 82 |
| 154 | 66 84 |
| 155 | 66 86 |
| 156 | 66 88 |
| 157 | 68 77 |
| 158 | 68 82 |
| 159 | 68 84 |
| 160 | 68 86 |
| 161 | 68 88 |
| 162 | 77 82 |
| 163 | 77 84 |
| 164 | 77 86 |
| 165 | 77 88 |
| 166 | 82 84 |
| 167 | 82 86 |
| 168 | 82 88 |
| 169 | 84 86 |
| 170 | 84 88 |
| 171 | 86 88 |

Results
Low Concentration of GM1 OS

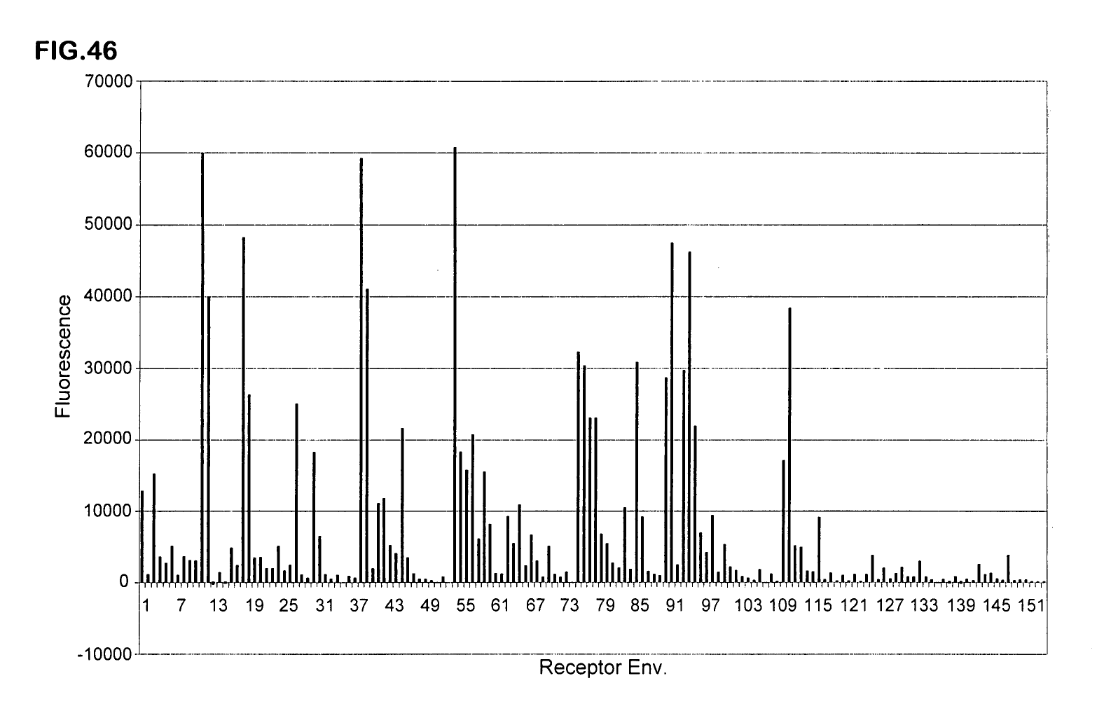

FIG. 46 illustrates binding of cholera toxin to the microarray of candidate artificial receptors followed by washing with buffer produced fluorescence signals. These fluorescence signals demonstrate that the cholera toxin bound strongly to certain receptor environments, weakly to others, and undetectably to some. Comparison to experiments including those reported in Example 2 indicates that cholera toxin binding was reproducible from array to array and from month to month.

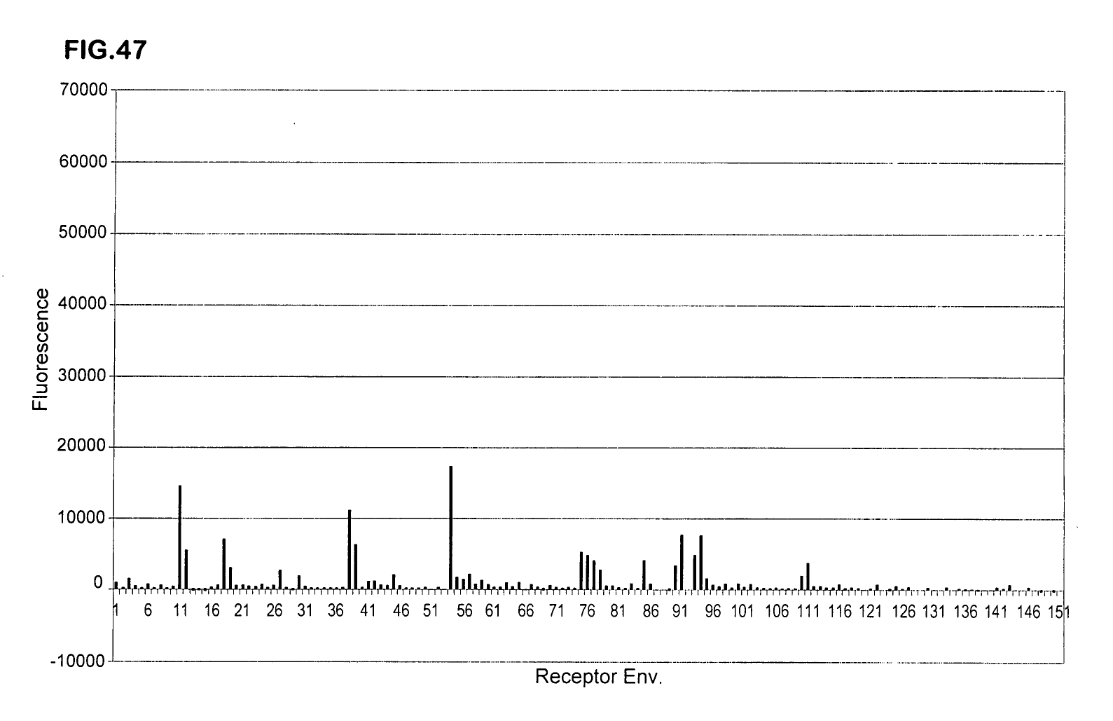

Binding of cholera toxin was also conducted with competition from GM1 OS (0.34 μM). FIG. 47 illustrates the fluorescence signals due to cholera toxin binding that were detected after this competition. Notably, many of the signals illustrated in FIG. 47 are significantly smaller than the corresponding signals recorded in FIG. 46. The small signals observed in FIG. 47 represent less cholera toxin bound to the array. GM1 OS significantly disrupted binding of cholera toxin to many of the receptor environments.

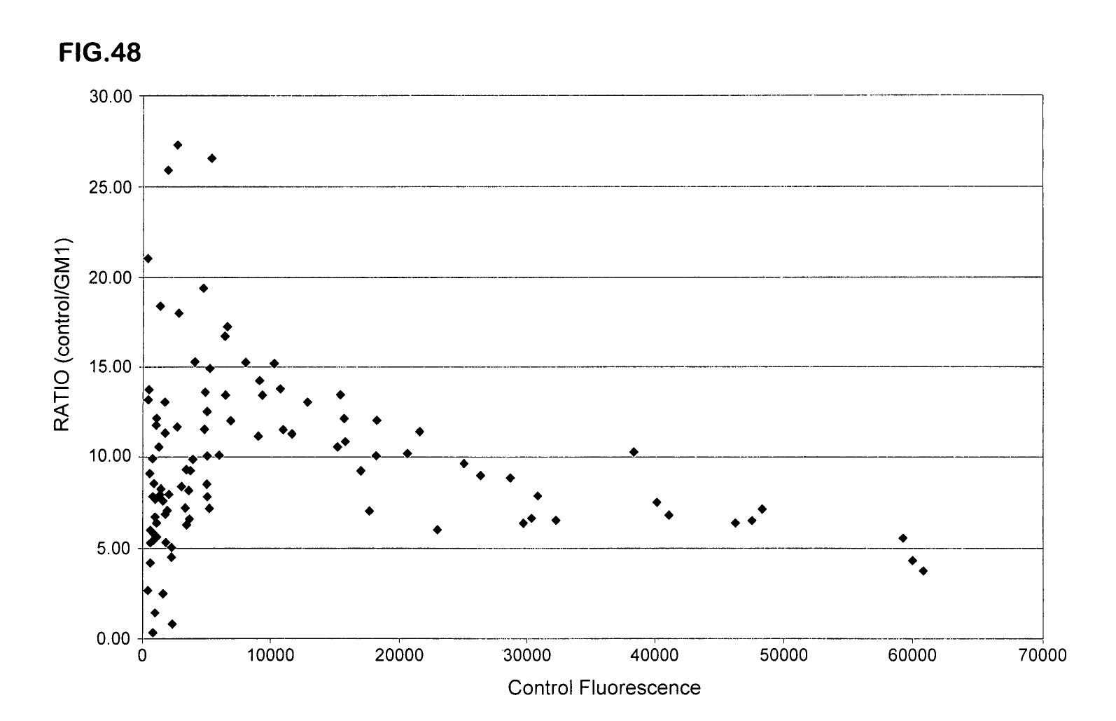

The disruption in cholera toxin binding caused by GM1 OS can be visualized as the ratio of the amount bound in the absence of GM1 OS to the amount bound in competition with GM1 OS. This ratio is illustrated in FIG. 48. The larger the ratio, the less cholera toxin remained bound to the artificial receptor after competition with GM1 OS. The ratio can be as large as about 30. The ratios are independent of the quantity bound in the control.

High Concentration of GM1 OS

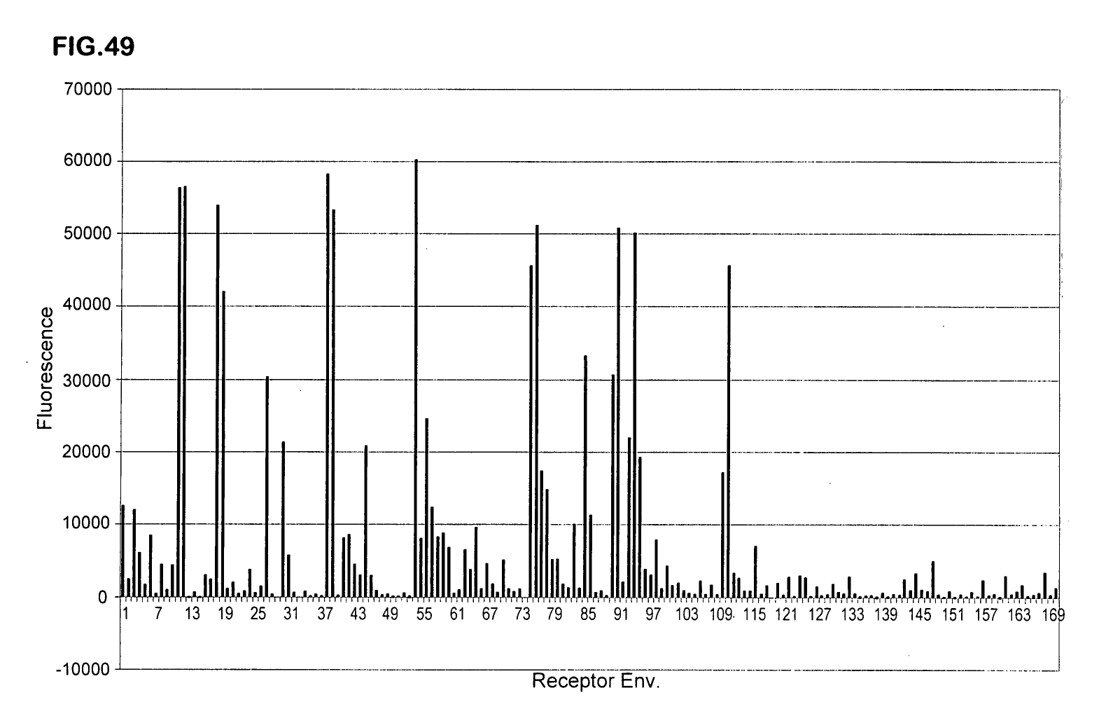

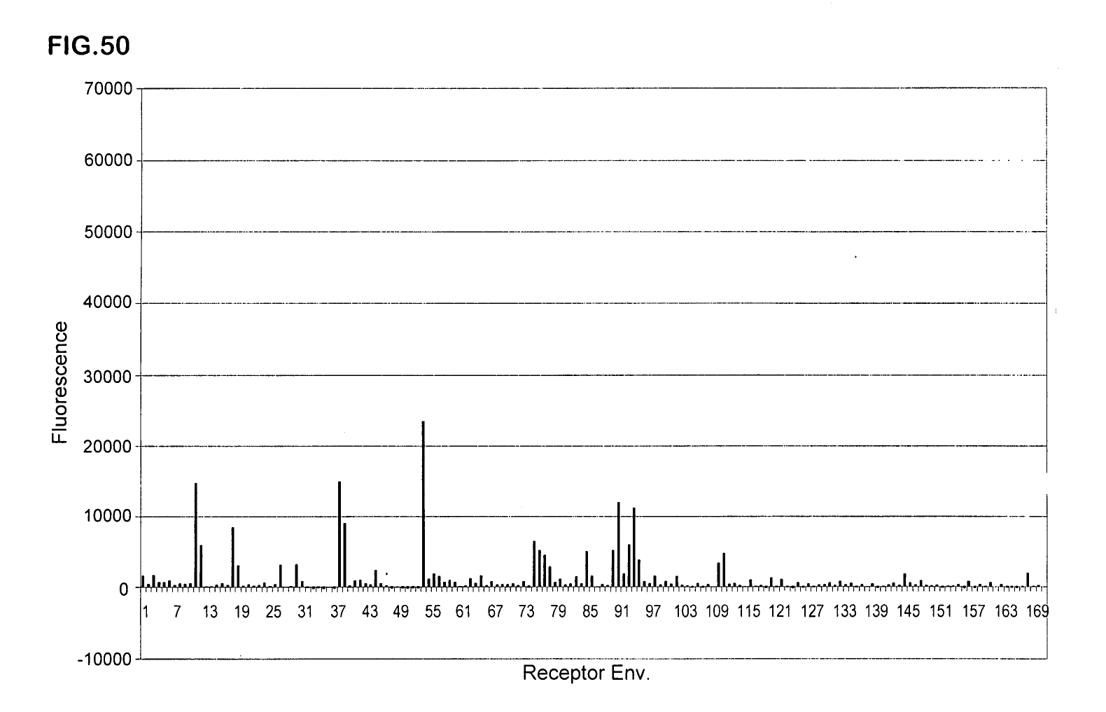

Binding of cholera toxin to the microarray of candidate artificial receptors followed by washing with buffer produced fluorescence signals illustrated in FIG. 49. As before, cholera toxin was reproducible and it bound strongly to certain receptor environments, weakly to others, and undetectably to some. FIG. 50 illustrates the fluorescence signals detected due to cholera toxin binding that were detected upon competition with GM1 OS at 5.1 μM. Again, GM1 OS significantly disrupted binding of cholera toxin to many of the receptor environments.

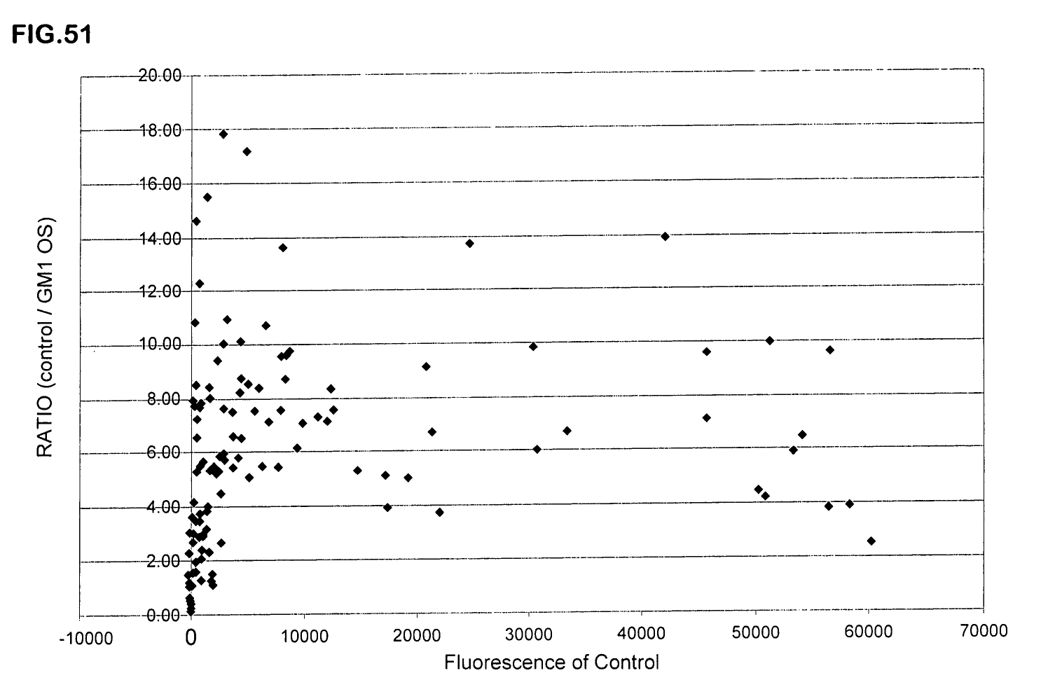

This disruption is presented as the ratio of the amount bound in the absence of GM1 OS to the amount bound after contacting with GM1 OS in FIG. 51. The ratios range up to about 18 and are independent of the quantity bound in the control.

Conclusions

This experiment demonstrated that binding of a test ligand to an artificial receptor of the present invention can be diminished (e.g., competed) by a candidate disruptor molecule. In this case the test ligand was the protein cholera toxin and the candidate disrupter was a compound known to bind to cholera toxin, GM1 OS. The degree to which binding of the test ligand was disrupted was independent of the degree to which the test ligand bound to the artificial receptor.

### Example 5

### GM1 Competes with Artificial Receptors for Binding to Cholera Toxin

Microarrays of candidate artificial receptors were made and evaluated for binding of cholera toxin. The arrays were also evaluated for disrupting that binding. Disrupting of binding employed a compound that binds to cholera toxin, the liposaccharide GM1. The results obtained demonstrate that a ligand of a protein specifically disrupts binding of the protein to the microarray.

Materials and Methods

Building blocks were synthesized and activated as described in Example 1. The building blocks employed in this example were TyrA1B1 [1-1], TyrA2B2, TyrA2B4, TyrA2B6, TyrA4B2, TyrA4B4, TyrA4B6, TyrA6B2, TyrA6B4, and TyrA6B6 in groups of 4 building blocks per artificial receptor. The abbreviation for the building block including a linker, a tyrosine framework, and recognition elements AxBy is TyrAxBy.

Microarrays for the evaluation of the 126 n=4 candidate receptor environments were prepared as described above for Example 4. The test ligand employed in these experiments was cholera toxin labeled with the Alexa™ fluorophore (Molecular Probes Inc., Eugene, Oreg.). Cholera toxin was employed at 5.3 nM in both the control and the competition experiments. The candidate disrupter employed in these experiments was GM1, a known ligand for cholera toxin, which competed at concentrations of 0.042, 0.42, and 8.4 μM. Microarray incubation and analysis was conducted as described for Example 4.

Table 3 identifies the building blocks in each receptor environment.

TABLE 3

| | Building |
|---|---|
| | Blocks |
| 1 | 22 24 26 42 |
| 2 | 22 24 26 44 |
| 3 | 22 24 26 46 |
| 4 | 22 24 26 61 |
| 5 | 22 24 26 64 |
| 6 | 22 24 26 66 |
| 7 | 22 24 42 44 |
| 8 | 22 24 42 46 |
| 9 | 22 24 42 62 |
| 10 | 22 24 42 46 |
| 11 | 22 24 42 66 |
| 12 | 22 24 44 46 |
| 13 | 22 24 44 62 |
| 14 | 22 24 44 64 |
| 15 | 22 24 44 66 |
| 16 | 22 24 46 62 |
| 17 | 22 24 46 64 |
| 18 | 22 24 46 66 |
| 19 | 22 24 62 64 |
| 20 | 22 24 62 66 |
| 21 | 22 24 64 66 |
| 22 | 22 26 42 44 |
| 23 | 22 26 42 46 |
| 24 | 22 26 42 62 |
| 25 | 22 26 42 64 |
| 26 | 22 26 42 66 |
| 27 | 22 26 44 46 |
| 28 | 22 26 44 62 |
| 29 | 22 26 44 64 |
| 30 | 22 26 44 66 |
| 31 | 22 26 46 62 |
| 32 | 22 26 46 64 |
| 33 | 22 26 46 66 |
| 34 | 22 26 62 64 |
| 35 | 22 26 62 66 |
| 36 | 22 26 64 66 |
| 37 | 22 42 44 46 |
| 38 | 22 42 44 62 |
| 39 | 22 42 44 64 |
| 40 | 22 42 44 66 |
| 41 | 22 42 46 62 |
| 42 | 22 42 46 64 |
| 43 | 22 42 46 66 |
| 44 | 22 42 62 64 |
| 45 | 22 42 62 66 |
| 46 | 22 42 64 66 |
| 47 | 22 44 46 62 |
| 48 | 22 44 46 64 |
| 49 | 22 44 46 66 |
| 50 | 22 44 62 64 |
| 51 | 22 44 62 66 |
| 52 | 22 44 64 66 |
| 53 | 22 46 62 64 |
| 54 | 22 46 62 66 |
| 55 | 22 46 64 66 |
| 56 | 22 62 64 66 |
| 57 | 24 26 42 44 |
| 58 | 24 26 42 46 |
| 59 | 24 26 42 62 |
| 60 | 24 26 42 64 |
| 61 | 24 26 42 66 |
| 62 | 24 26 44 46 |
| 63 | 24 26 44 62 |
| 64 | 24 26 44 64 |
| 65 | 24 26 44 66 |
| 66 | 24 26 46 62 |
| 67 | 24 26 46 64 |
| 68 | 24 26 46 66 |
| 69 | 24 26 62 64 |
| 70 | 24 26 62 66 |
| 71 | 24 26 64 66 |
| 72 | 24 42 44 46 |
| 73 | 24 42 44 62 |
| 74 | 24 42 44 64 |
| 75 | 24 42 44 66 |
| 76 | 24 42 46 62 |
| 77 | 24 42 46 64 |
| 78 | 24 42 46 66 |
| 79 | 24 42 62 64 |
| 80 | 24 42 62 66 |
| 81 | 24 42 64 66 |
| 82 | 24 44 46 62 |
| 83 | 24 44 46 64 |
| 84 | 24 44 46 66 |
| 85 | 24 44 62 64 |
| 86 | 24 44 62 66 |
| 87 | 24 44 64 66 |
| 88 | 24 46 62 64 |
| 89 | 24 46 62 66 |
| 90 | 24 46 64 66 |
| 91 | 24 62 64 66 |
| 92 | 26 42 44 46 |
| 93 | 26 42 44 62 |
| 94 | 26 42 44 64 |
| 95 | 26 42 44 66 |
| 96 | 26 42 46 62 |
| 97 | 26 42 46 64 |
| 98 | 26 42 46 66 |
| 99 | 26 42 62 64 |
| 100 | 26 42 62 66 |
| 101 | 26 42 64 66 |
| 102 | 26 44 46 62 |
| 103 | 26 44 46 64 |
| 104 | 26 44 46 66 |
| 105 | 26 44 62 64 |
| 106 | 26 44 62 66 |
| 107 | 26 44 64 66 |
| 108 | 26 46 62 64 |
| 109 | 26 46 62 66 |
| 110 | 26 46 64 66 |
| 111 | 26 62 64 66 |
| 112 | 42 44 46 62 |
| 113 | 42 44 46 64 |
| 114 | 42 44 46 66 |
| 115 | 42 44 62 64 |
| 116 | 42 44 62 66 |
| 117 | 42 44 64 66 |
| 118 | 42 46 62 64 |
| 119 | 42 46 62 66 |
| 120 | 42 46 64 66 |
| 121 | 42 62 64 66 |
| 122 | 44 46 62 64 |
| 123 | 44 46 62 66 |
| 124 | 44 46 64 66 |
| 125 | 44 62 64 66 |
| 126 | 46 62 64 66 |

Results

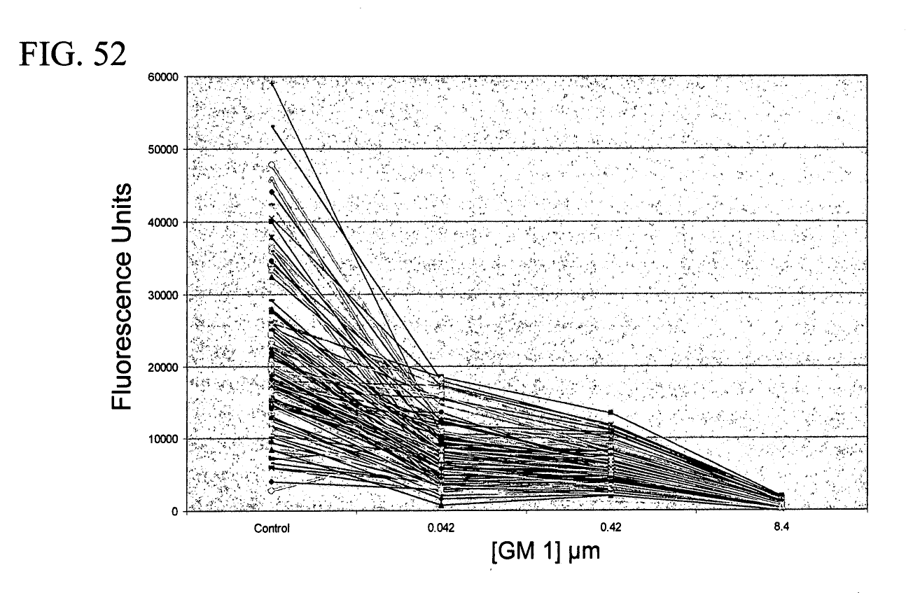

FIG. 52 illustrates the fluorescence signals produced by binding of cholera toxin to the microarray of candidate artificial receptors alone and in competition with each of the three concentrations of GM1. The magnitude of the fluorescence signal decreases steadily with increasing concentration of GM1. The amount of decrease is not quantitatively identical for all of the receptors, but each receptor experienced decreased binding of cholera toxin. These decreases indicate that GM1 competed with the artificial receptor for binding to the cholera toxin.

The decreases show a pattern of relative competition for the binding site on cholera toxin. This can be demonstrated through graphs of fluorescence signal obtained at a particular concentration of GM1 against fluorescence signal in the absence of GM1 (not shown). Certain of the receptors appear at similar relative positions on these plots as concentration of GM1 increases.

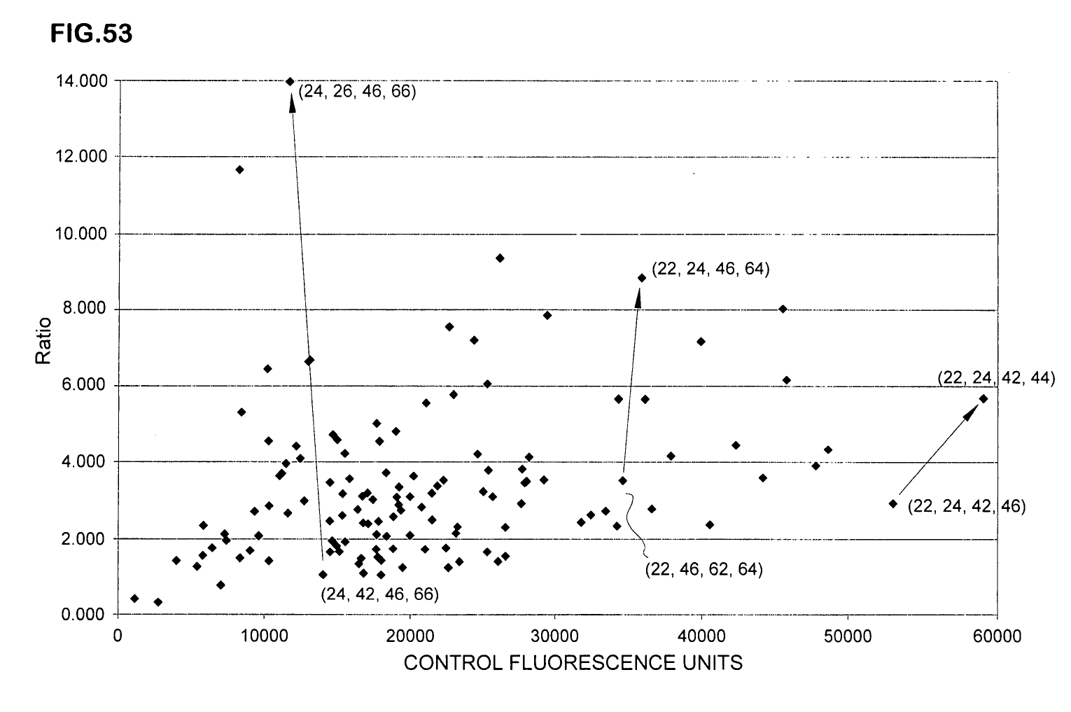

The disruption in cholera toxin binding caused by GM1 can be visualized as the ratio of the amount bound in the absence of GM1 OS to the amount bound upon competition with GM1. This ratio is illustrated in FIG. 53. The larger the ratio, the more cholera toxin remained bound to the artificial receptor upon competition with GM1. The ratio can be as large as about 14. The ratios are independent of the quantity bound in the control.

Interestingly, in several instances minor changes in structure to the artificial receptor caused significant changes in the ratio. For example, the artificial receptor including building blocks 24, 26, 46, and 66 differs from that including 24, 42, 46, and 66 by only substitution of a single building block. (xy indicates building block TyrAxBy.) The substitution of building block 42 for 26 increased binding in the presence of GM1 by about 14-fold.

By way of further example, the artificial receptor including building blocks 22, 24, 46, and 64 differs from that including 22, 46, 62, and 64 by only substitution of a single building block. The substitution of building block 24 for 62 increased binding in the presence of GM1 by about 3-fold.

Even substitution of a single recognition element affected binding. The artificial receptor including building blocks 22, 24, 42, and 44 differs from that including 22, 24, 42, and 46 by only substitution of a single recognition element. The substitution of building block 44 for 46 (a change of recognition element B6 to B4) increased binding in the presence of GM1 by about 3-fold.

Conclusions

This experiment demonstrated that binding of a test ligand to an artificial receptor of the present invention can be diminished (e.g., competed) by a candidate disruptor molecule. In this case the test ligand was the protein cholera toxin and the candidate disrupter was a compound known to bind to cholera toxin, GM1. Minor changes in structure of the building blocks making up the artificial receptor caused significant changes in the competition.

### Example 6

### GM1 Employed as a Building Block Alters Binding of Cholera Toxin to the Present Artificial Receptors

Microarrays of candidate artificial receptors were made, GM1 was bound to the arrays, and they were evaluated for binding of cholera toxin. The results obtained demonstrate that adding GM1 as a building block in an array of artificial receptors can increase binding to certain of the receptors.

Materials and Methods

Building blocks were synthesized and activated as described in Example 1. The building blocks employed in this example were those described in Example 4. Microarrays for the evaluation of the 171 n=2 candidate receptor environments were prepared as described above for Example 4. The test ligand employed in these experiments was cholera toxin labeled with the Alexa™ fluorophore (Molecular Probes Inc., Eugene, Oreg.). Cholera toxin was employed at 0.01 ug/ml (0.17 pM) or 0.1 ug/ml (1.7 pM) in both the control and the competition experiments. GM1 was employed as a test ligand for the artificial receptors and became a building block for receptors used to bind cholera toxin. The arrays were contacted with GM1 at either 100 μg/ml, 10 μg/ml, or 1 μg/ml as described above for cholera toxin and then rinsed with deionized water. The arrays were then contacted with cholera toxin under the conditions described above. Microarray analysis was conducted as described for Example 4. Table 2 identifies the building blocks in each receptor environment.

Results

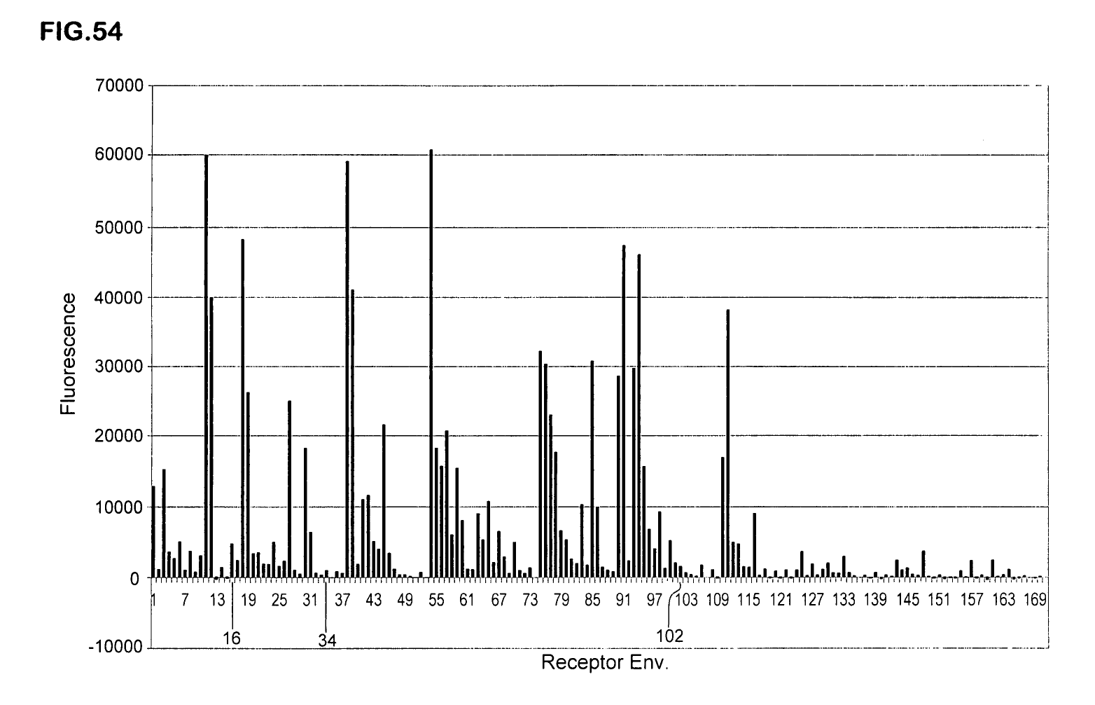

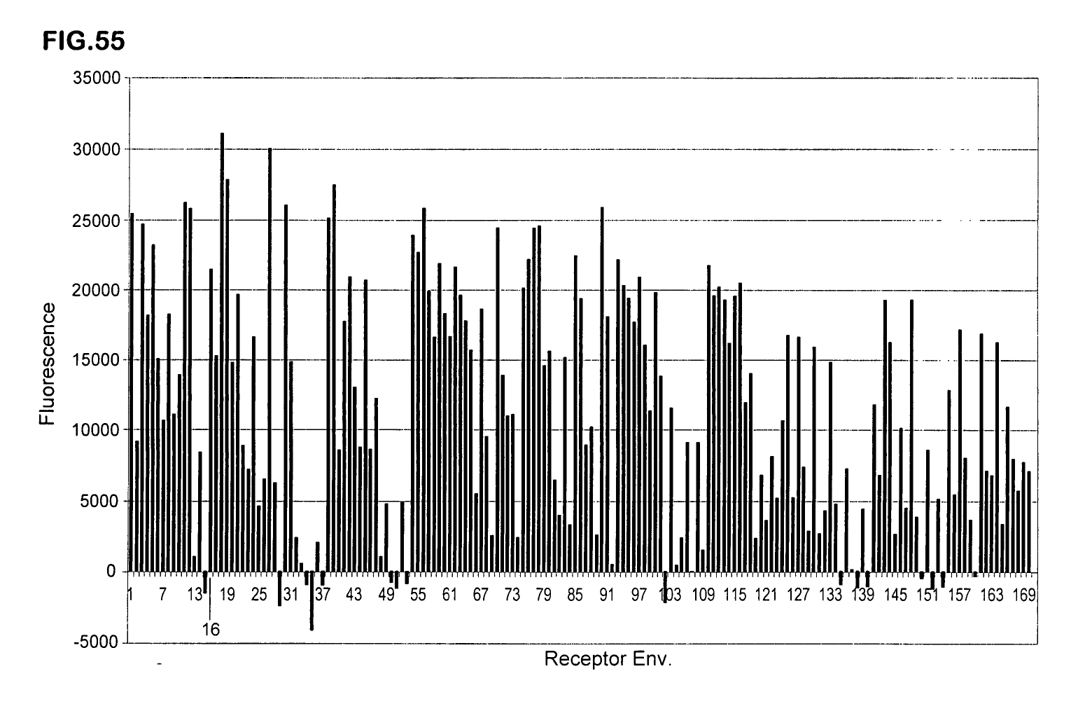

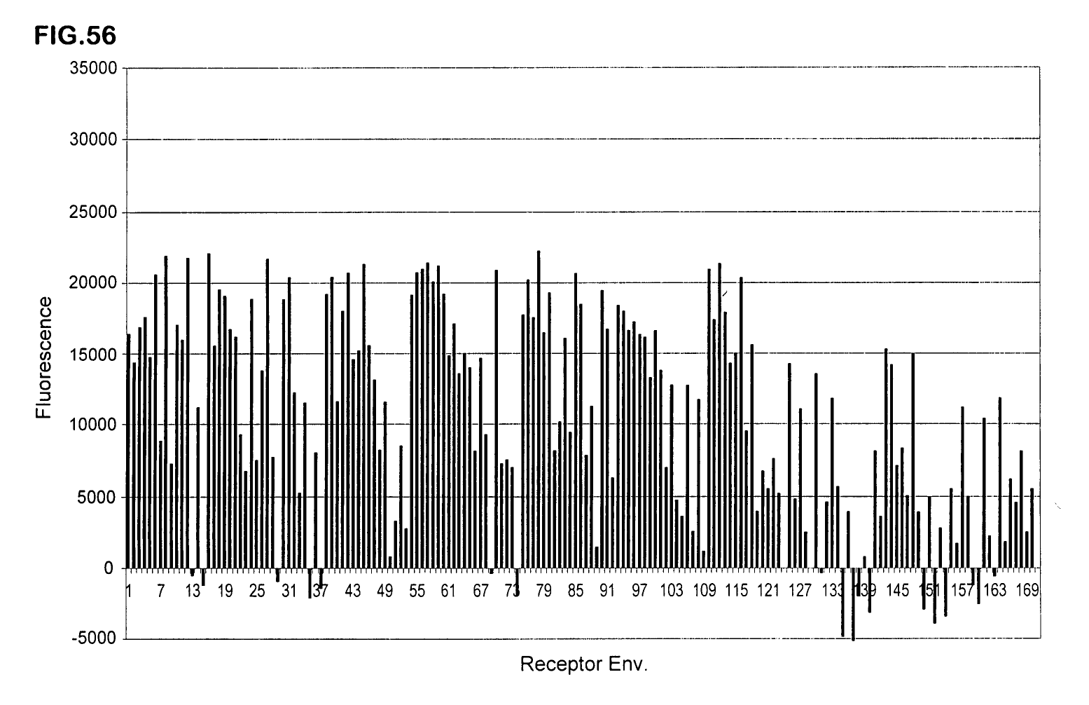

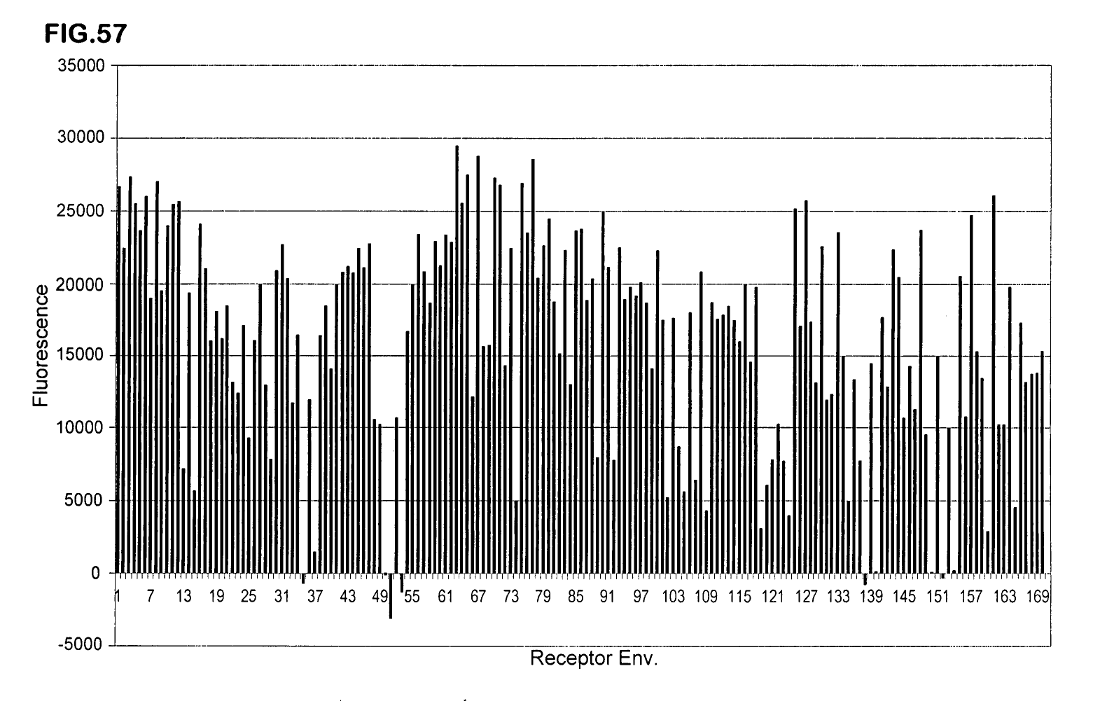

FIG. 54 illustrates the fluorescence signals produced by binding of cholera toxin to the microarray of candidate artificial receptors without pretreatment with GM1. Binding of GM1 to the microarray of candidate artificial receptors followed by binding of cholera toxin produced fluorescence signals illustrated in FIGS. 55, 56, and 57 (100 μg/ml, 10 μg/ml, and 1 μg/ml GM1, respectively).

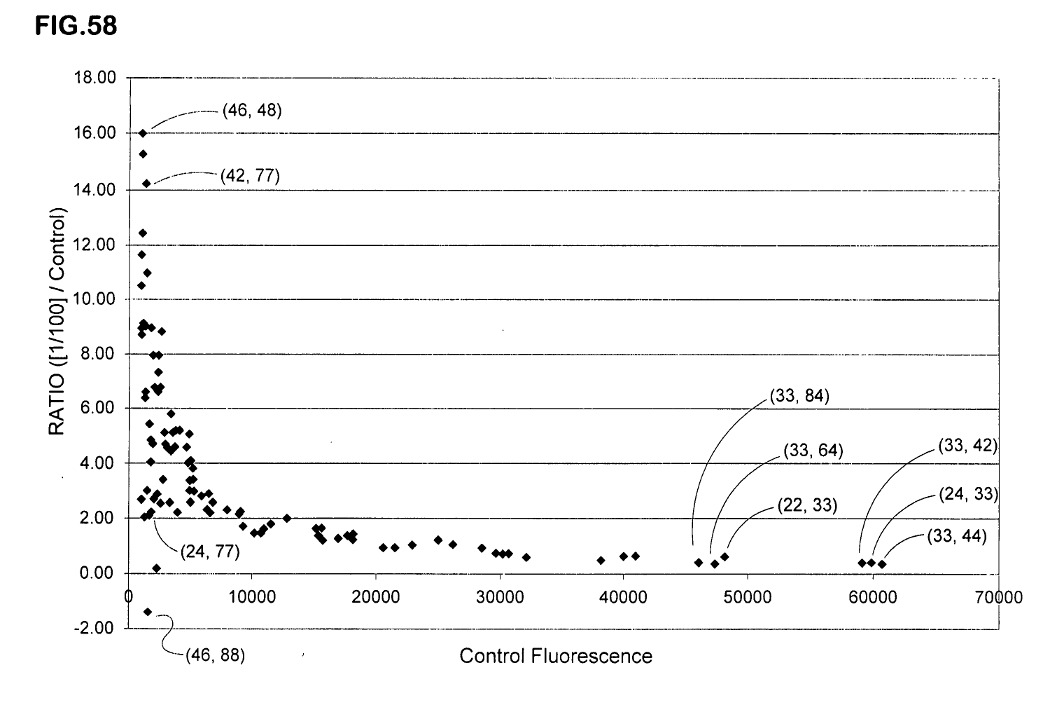

The enhancement of cholera toxin binding caused by pretreatment with GM1 can be visualized as the ratio of the amount bound in the presence of GM1 to the amount bound in the absence of GM1. This ratio is illustrated in FIG. 58 for 1 μg/ml GM1. The larger the ratio, the more cholera toxin bound to the artificial receptor after pretreatment with GM1. The ratio can be as large as about 16.

In several instances minor changes in structure to the artificial receptor caused significant changes in the ratio. For example, the artificial receptor including building blocks 46 and 48 differs from that including 46 and 88 by only substitution of a single recognition element on a single building block. (xy indicates building block TyrAxBy.) The substitution of building block 48 for 88 (a change of recognition element A8 to A4) increased the ratio representing increased binding the presence of GM1 building block from about 0.5 to about 16. Similarly, the artificial receptor including building blocks 42 and 77 differs from that including 24 and 77 by only substitution of a single building block. The substitution of building block 42 for 24 increased the ratio representing increased binding the presence of GM1 building block from about 2 to about 14.

Interestingly, several building blocks that exhibited high levels of binding of cholera toxin (signals of 45,000 to 65,000 fluorescence units) and that include the building block 33 were not strongly affected by the presence of GM1 as a building block.

Conclusions

This experiment demonstrated that binding of GM1 to an artificial receptor of the present invention can significantly increase binding by cholera toxin. Minor changes in structure of the building blocks making up the artificial receptor caused significant changes in the degree to which GM1 enhanced binding of cholera toxin.

Discussion of Examples 4-6

We have previously demonstrated that an array of working artificial receptors bind to a protein target in a manner which is complementary to the specific environment presented by each region of the proteins surface topology. Thus the pattern of binding of a protein target to an array of working artificial receptors describes the proteins surface topology; including surface structures which participate in e.g., protein˜small molecule, protein˜peptide, protein-protein, protein˜carbohydrate, protein˜DNA, etc. interactions. It is thus possible to use the binding of a selected protein to a working artificial receptor array to characterize these protein˜small molecule, protein˜peptide, protein-protein, protein˜carbohydrate, protein˜DNA, etc. interactions. Moreover, it is possible to utilize the protein to array interactions to define “leads” for the disruption of these interactions.

Cholera Toxin B sub-unit binds to GM1 on the cell surface. Studies to identify competitors to this binding event have shown that competitors to the cholera toxin: GM1 binding interaction (binding site) can utilize both a sugar and an alkyl/aromatic functionality (Pickens, et al., Chemistry and Biology, vol. 9, pp 215-224 (2002)). We have previously demonstrated that fluorescently labeled Cholera Toxin B sub-unit binds to arrays of the present artificial receptors to give a defined binding pattern which reflects cholera toxin B's surface topology. For this study, we sought to demonstrate that the binding of the cholera toxin to at least some members of the array could be disrupted using cholera toxin's natural ligand, GM1.

The results presented in the figures clearly demonstrate that these goals have been achieved. Specifically, competition between the GM1 OS pentasaccharide or GM1 and an artificial receptor array for cholera binding clearly gave a binding pattern which was distinct from the cholera binding pattern control. Moreover, these results demonstrated the complementarity between several of the working artificial receptors which contained a naphthyl moiety when compared to working artificial receptors which only contained phenyl functionality. These results are in keeping with the active site competition studies in Pickens, et al. and indicate that the naphthyl and phenyl derivatives represent good mimics/probes for the cholera to GM1 interaction. The specificity of these interactions was demonstrated by the observation that the change of a single building block out of 4 in a combination of 4 building blocks system changed a non-competitive to a significantly competitive environment. These results also indicated that selected working artificial receptors can be used to develop a high-throughput screen for the further evaluation of the cholera:GM1 interaction.

Additionally, we sought to demonstrate that an affinity support/membrane mimic could be prepared by pre-incubating an array of artificial receptors with GM1 which would then bind/capture cholera toxin in a binding pattern which could be used to select a working artificial receptor(s) for, for example, the high-throughput screen of lead compounds which will disrupt the “cholera:membrane˜GM1 mimic”. The GM1 pre-incubation studies clearly demonstrated that several of the working artificial receptors which were poor cholera binders significantly increased their cholera binding, presumably through an affinity interaction between the cholera toxin and both the immobilized GM1 pentasaccharide moiety and the working artificial receptor building block environment.

### Example 7

### Gradients of Building Blocks Bind and Distinguish Cholera Toxin and Phycoerythrin

Gradients of the artificial receptors were made and protein was flowed along the gradient. The gradients were evaluated for binding of cholera toxin and/or phycoerythrin. The results obtained demonstrate that gradients of the artificial receptors bind and distinguish proteins, such as cholera toxin and phycoerythrin.

Materials and Methods

Gradients of the present artificial receptors were constructed using known methods for making gradients of molecules along a surface. Two types of gradients were prepared, step gradients and continuous gradients.

The step gradients were prepared by methods employed for coupling building blocks to surfaces in regions or arrays. Briefly, a stock solution of each building block or mixture of building blocks was prepared at 15 mg/ml in DMF/H₂O/PEG400 (90/10/10, v/v/v), some also included dimethylaminopyridine. This stock solution was diluted 1/10, 1/20, 1/40, 1/80, 1/160, and 1/320 for applying to amine functionalized slides (ArrayIt SuperAmine). The diluted solutions of building blocks were applied to the slides in stripes (steps) across the shorter dimension of the slide. After applying the building blocks to the slide, the slide was allowed to sit for 60 minutes at room temperature. The slides were then washed with EtOH/H₂O (20/80 v/v, adjusted to pH about 2 with 1N HCl) followed by deionized water, dried using a stream of compressed air, and stored at room temperature. The slides were further modified by reacting the remaining amines with acetic anhydride to form an acetamide lawn in place of the amine lawn. In the experiments reported in this Example, the stripes (steps) of building blocks on the slide were separated by stripes of acetamide lawn. In other experiments, the steps of building blocks were contiguous.

The continuous gradients were prepared by known methods for preparing surface-bound molecular gradients (Kramer et al., J. Amer. Chem. Soc. 126: 5388-5395 (2004) and references therein). Briefly, an amine functionalized slide was placed in a 150 ml beaker so that it was standing on one end against the wall of the beaker. A solution of building block as described above at the 1/80 or 1/120 dilution was introduced into the beaker at a rate of 2 ml/min over a period of 60 minutes. In this manner, the bottom of the slide was in contact with the building block solution for a longer time than the top of the slide. Thus, the bottom of the slide included a higher density of coupled building blocks than the top.

Proteins (e.g., cholera toxin and/or phycoerythrin) were contacted with the gradients by flowing protein solutions down the length of the slide in a small trough or flow-cartridge. The cholera toxin was labeled with the Alexa™ fluorophore (Molecular Probes Inc., Eugene, Oreg.). The flow-cartridge included an inlet for protein solution at one end and an outlet at the other. A total of 2 ml of protein solution was run through the flow-cartridge having a volume of about 1.8 ml over 5 or 120 minutes at room temperature followed by rinsing with 2 ml of PBS-T and with 10 ml of deionized water. Phycoerythrin was at 0.2 or 2 μg/ml. Cholera toxin was at 0.01 or 0.1 μg/ml.

Results

FIGS. 59, 61, 63, 65, 67, 69, 71, 73, 75, 77, and 79 show fluorescence images of gradient slides after protein has been flowed over them. In each of these Figures, the concentration of the building block increased in steps from the top of each slide to the bottom. The protein flowed in a direction shown as from top to bottom in the Figure, although the slide was horizontal during flow.

FIGS. 60, 62, 64, 66, 68, 70, 72, 74, 76, 78, and 80 show fluorescence intensity plots corresponding to the images. In each of these Figures, the concentration of the building block increased in steps from right to left. The protein flowed from left to right (unless described otherwise).

FIG. 59 presents an image obtained from a run of phycoerythrin over a step gradient of increasing concentrations of the building block TyrA₃B₃. The image as reproduced shows fluorescence obtained from binding of phycoerythrin to at least receptor surfaces including the three highest concentration (the 4th, 5^{th}, and 6^{th}) steps of this building block. FIG. 60 shows increasing peaks of fluorescence for the 3^{rd}, 4^{th}, 5^{th}, and 6^{th} steps of the building block gradient. Phycoerythrin bound to receptors made up of building block TyrA₃B₃ and bound to a greater extent to receptors including higher concentrations of this building block.

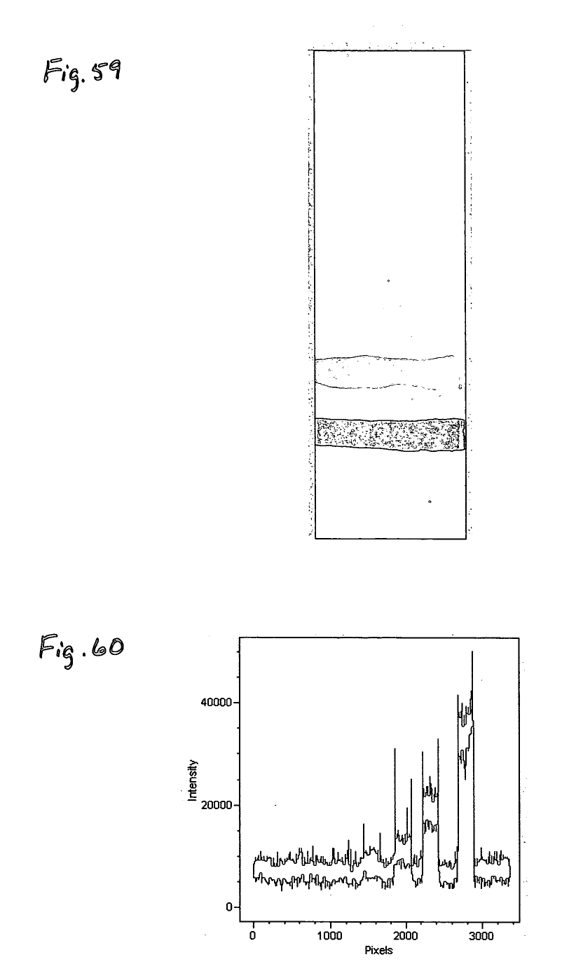

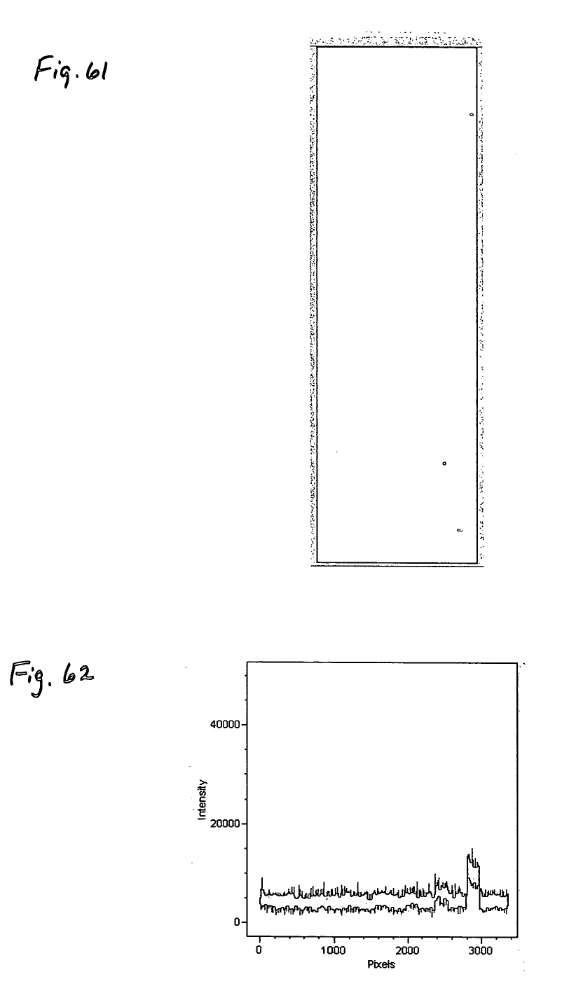

FIG. 61 presents an image obtained from a run of phycoerythrin over a step gradient of increasing concentrations of the building block TyrA₄B₄. The image as reproduced shows fluorescence obtained from binding of phycoerythrin to at least receptor surfaces including the highest concentration (the 6^{th}) step of this building block. FIG. 62 shows increasing peaks of fluorescence for the 5^{th} and 6^{th} steps of the building block gradient. Phycoerythrin bound to receptors made up of building block TyrA₄B₄ and bound to a greater extent to receptors including higher concentrations of this building block.

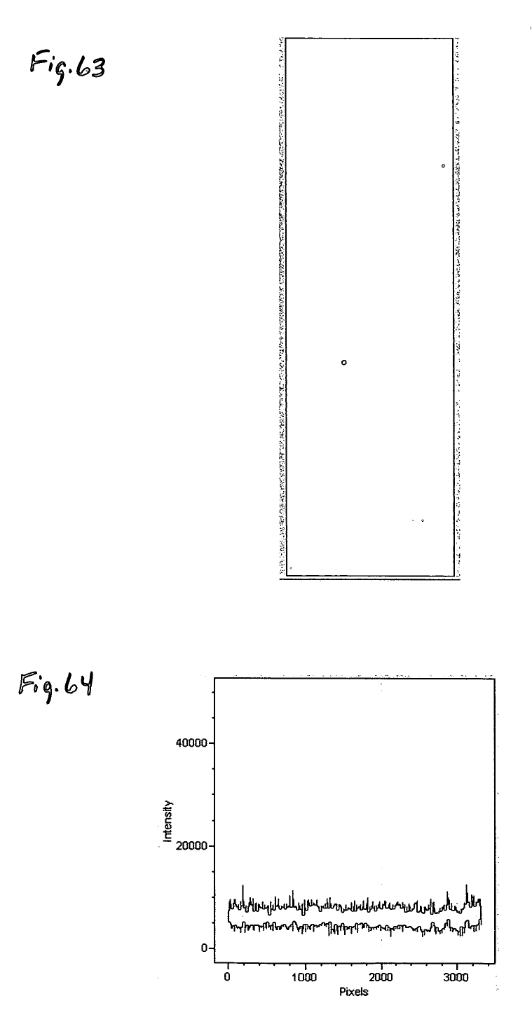

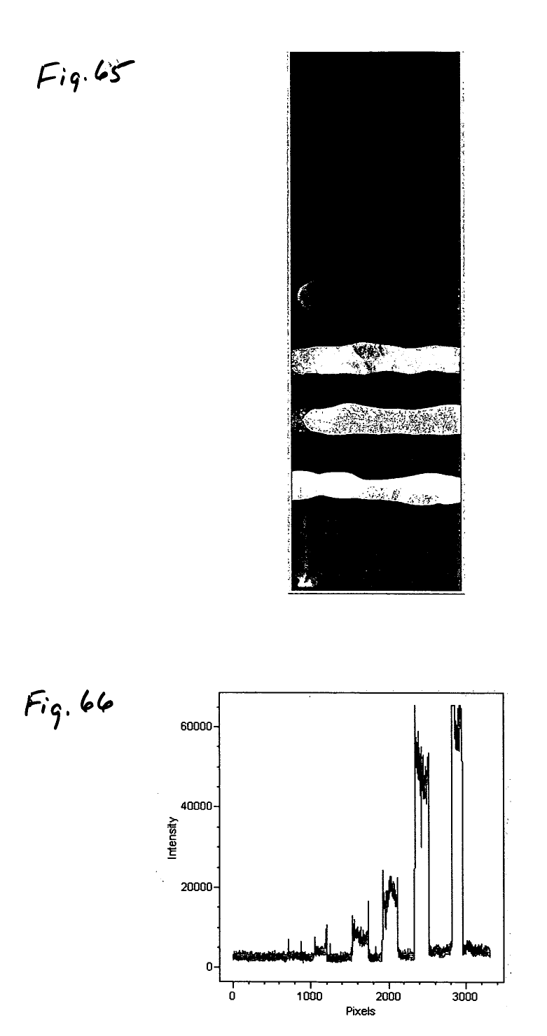

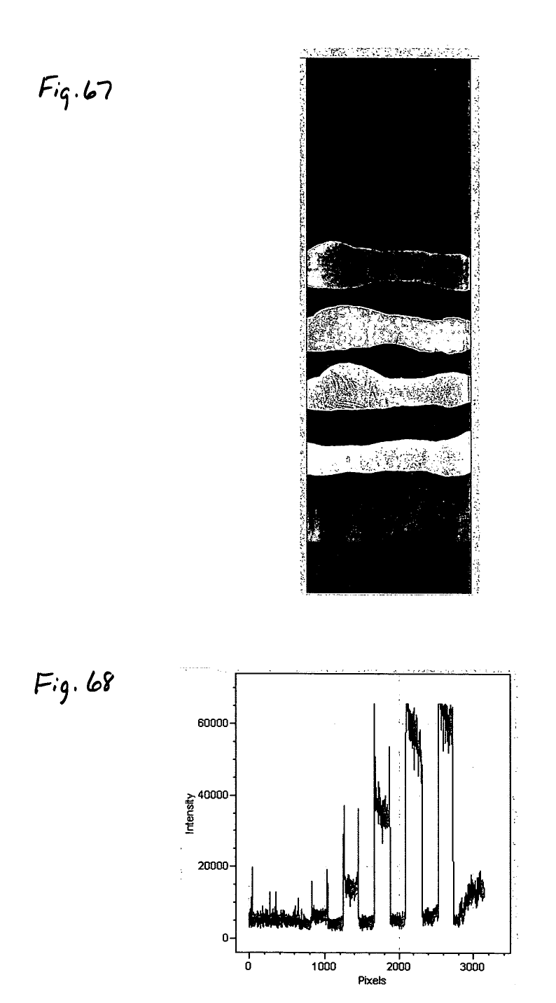

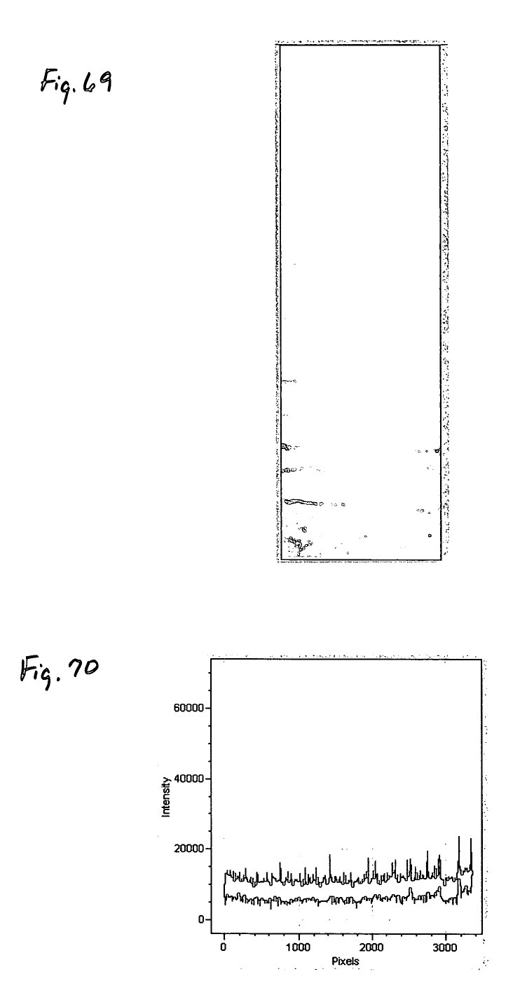

FIG. 63 presents an image obtained from a run of phycoerythrin over a step gradient of increasing concentrations of the building block TyrA₅B₅. The image as reproduced shows that phycoerythrin did not bind to any step of the gradient of this building block. The plot in FIG. 64 also illustrates that phycoerythrin did not bind to any step of the gradient of this building block.

FIG. 65 presents an image obtained from a run of cholera toxin over a step gradient of increasing concentrations of the building block TyrA₃B₃. The image as reproduced shows fluorescence obtained from binding of cholera toxin to at least receptor surfaces including the four highest concentration (the 3^{rd}, 4^{th}, 5^{th}, and 6^{th}) steps of this building block. FIG. 66 shows increasing peaks of fluorescence for the 2^{nd}, 3^{rd}, 4^{th}, 5^{th}, and 6^{th} steps of the building block gradient. Cholera toxin bound to receptors made up of building block TyrA₃B₃ and bound to a greater extent to receptors including higher concentrations of this building block.

FIG. 67 presents an image obtained from a run of cholera toxin over a step gradient of increasing concentrations of the building block TyrA₄B₄. The image as reproduced shows fluorescence obtained from binding of cholera toxin to at least receptor surfaces including the three highest concentration (the 4^{th}, 5^{th}, and 6^{th}) steps of this building block. FIG. 68 shows increasing peaks of fluorescence for at least the 2^{nd}, 3^{rd}, 4^{th}, 5^{th}, and 6^{th} steps of the building block gradient and possibly also for the first step. Cholera toxin bound to receptors made up of building block TyrA₄B₄ and bound to a greater extent to receptors including higher concentrations of this building block.

FIG. 69 presents an image obtained from a run of cholera toxin over a step gradient of increasing concentrations of the building block TyrA₅B₅. The image as reproduced shows that cholera toxin did not bind to any step of the gradient of this building block, but may have bound to the edges of several of the steps. This binding to the edges may be analogous to the “doughnut” effect sometimes observed in spots on microarrays. The plot in FIG. 70 also illustrates that cholera toxin did not bind to any step of the gradient of this building block.

FIG. 71 presents an image obtained from a run of cholera toxin over a step gradient of increasing concentrations of the building blocks TyrA₃B₃ and TyrA₄B₄ (in a 1:1 molar ratio). The cholera toxin was flowed across the slide from the lower concentration steps to the higher concentration steps. The image as reproduced shows fluorescence obtained from binding of cholera toxin to at least receptor surfaces including the three highest concentrations (the 4^{th}, 5^{th}, and 6^{th}) steps of these building blocks. FIG. 72 shows increasing peaks of fluorescence for the 3^{rd}, 4^{th}, 5^{th}, and 6^{th} steps of the building block gradient. Cholera toxin bound to receptors made up of building blocks TyrA₃B₃ and TyrA₄B₄ and bound to a greater extent to receptors including higher concentrations of these building blocks.

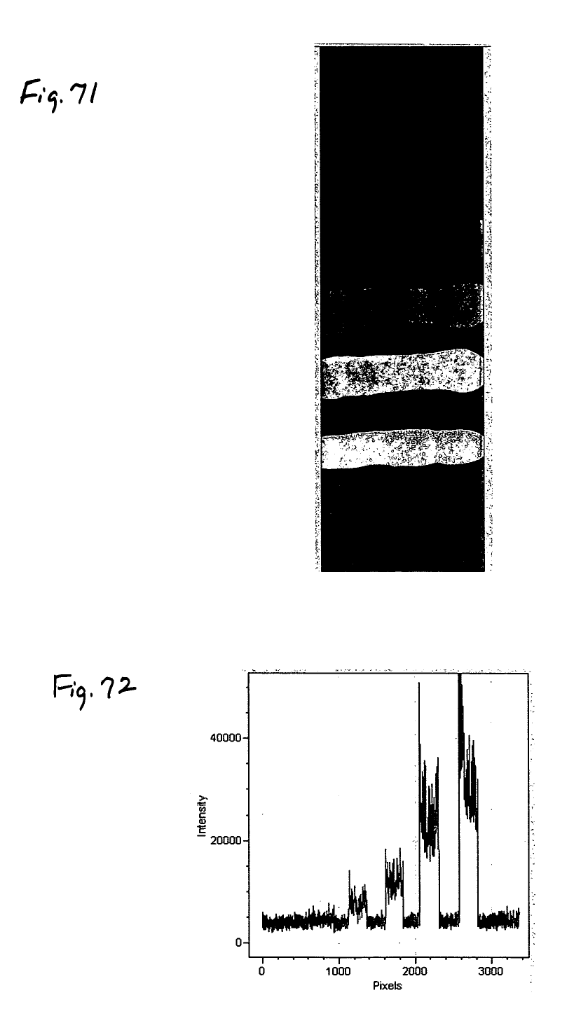

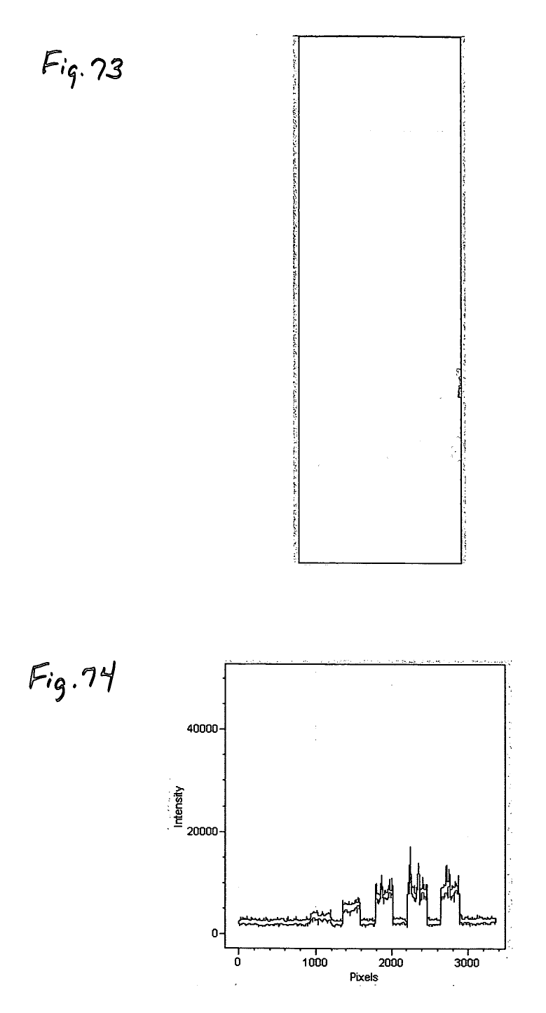

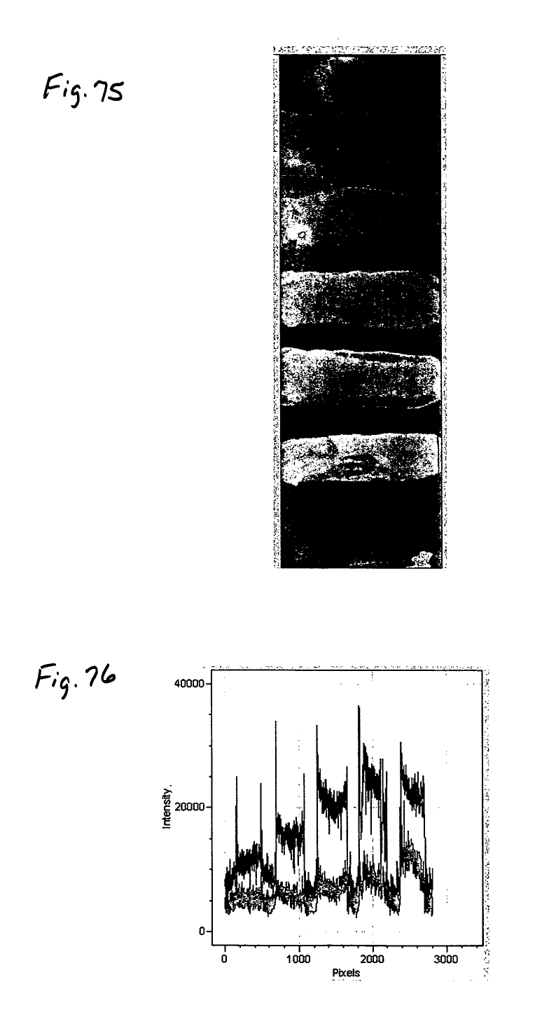

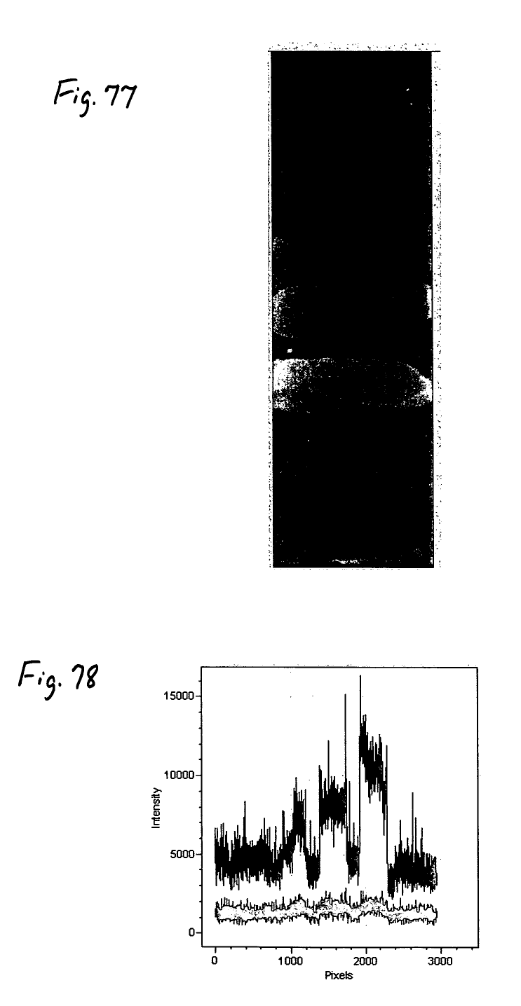

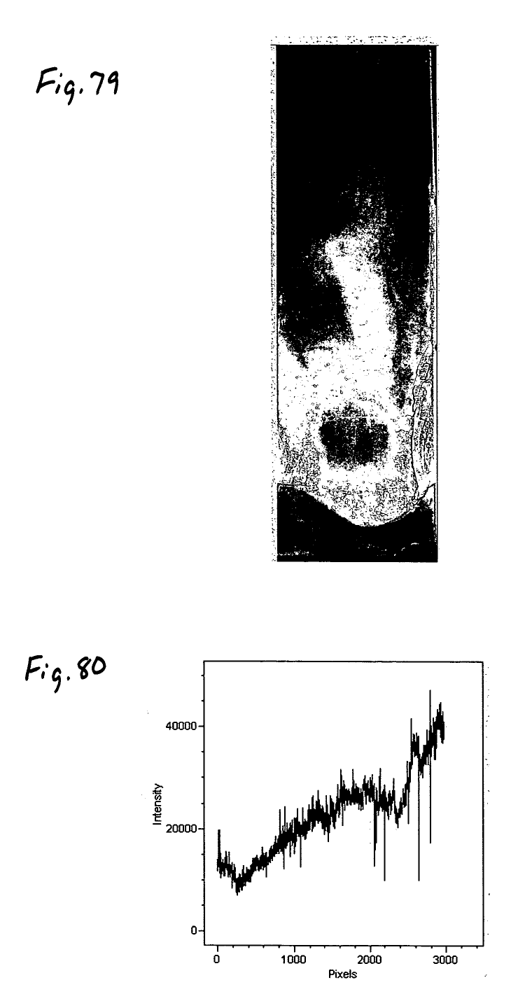

FIG. 73 presents an image obtained from a run of cholera toxin over a step gradient of increasing concentrations of the building blocks TyrA₃B₃ and TyrA₄B₄ (in a 1:1 molar ratio). The cholera toxin was flowed across the slide from the higher concentration steps to the lower concentration steps. The image as reproduced shows fluorescence obtained from binding of cholera toxin to at least receptor surfaces including the three highest concentration (the 6^{th}, 5^{th}, and 4^{th}) steps of this building block. FIG. 74 shows peaks of fluorescence for at least the 6^{th}, 5^{th}, 4^{th}, 3^{rd}, and 2^{nd} steps of the building block gradient.

This plot obtained from flow of protein from high to low concentration steps is different in appearance from those obtained with protein flow from low to high concentrations. The plot in FIG. 74 indicates that the cholera toxin first encountered and saturated the highest concentration step, then encountered and saturated the next highest concentration (5^{th}) step, subsequently encountered and saturated the next highest concentration (4^{th}) step, and also bound to the 3^{rd} and 2^{nd} steps.

Evaluating binding of cholera toxin and phycoerythrin to arrays of candidate artificial receptors identified receptors that preferentially bind cholera toxin rather than phycoerythrin (e.g., receptors including the building blocks TyrA₄B₄ and TyrA₄B₆). Such testing also identified receptors that bind both cholera toxin and phycoerythrin (e.g., receptors including the building blocks TyrA₃B₃ and TyrA₄B₄). These receptors were employed in step gradients to demonstrate selective binding of one protein compared to another on gradients of artificial receptors.

FIG. 75 presents an image obtained from a run of a mixture of cholera toxin and phycoerythrin over a step gradient of increasing concentrations of the building blocks TyrA₃B₃ and TyrA₄B₄ (in a 1:1 molar ratio). The gradient included five steps of increasing concentrations of the building blocks. The mixture included cholera toxin at 0.1 μg/ml and phycoerythrin at 2 μg/ml. The mixture flowed across the slide from the lower concentration steps to the higher concentration steps. The image as reproduced shows fluorescence obtained from binding of these proteins to at least receptor surfaces including the four highest concentration (2^{nd}, 3^{rd}, 4^{th}, and 5^{th}) steps of these building blocks.

FIG. 76 shows fluorescence intensities obtained for cholera toxin (top line) and phycoerythrin (bottom line). This plot shows increasing peaks of fluorescence for cholera toxin bound to at least the 2^{nd}, 3^{rd}, 4^{th}, and 5^{th} steps of the building block gradient. This plot shows increasing peaks of fluorescence for phycoerythrin bound to at least the 3^{rd}, 4^{th}, and 5^{th} steps of the building block gradient.

Gradients made up of receptors known to bind both cholera toxin and phycoerythrin, in fact, bound both of these proteins.

FIG. 77 presents an image obtained from a run of a mixture of cholera toxin and phycoerythrin over a step gradient of increasing concentrations of the building blocks TyrA₄B₄ and TyrA₄B₆ (in a 1:1 molar ratio). The gradient included five steps of increasing concentrations of the building blocks. The mixture included cholera toxin and phycoerythrin at concentrations as above. The mixture flowed across the slide from the lower concentration steps to the higher concentration steps. The image as reproduced shows fluorescence obtained from binding of cholera toxin to at least receptor surfaces including the three highest concentration (the 3^{rd}, 4^{th}, and 5^{th}) steps of these building blocks.

FIG. 78 shows fluorescence intensities obtained for cholera toxin (top line) and phycoerythrin (bottom line). This plot shows increasing peaks of fluorescence for cholera toxin bound to at least the 3^{rd}, 4^{th}, and 5^{th} steps of the building block gradient. This plot shows that phycoerythrin, as expected, did not bind to any steps of this building block gradient.

Gradients made up of receptors known to bind cholera toxin but not phycoerythrin, in fact, bound cholera toxin but not phycoerythrin.

FIG. 79 presents a fluorescence image from a run of cholera toxin flowed over a continuous gradient of increasing concentrations of the building blocks TyrA₃B₃ and TyrA₄B₄ (in a 1:1 molar ratio). The lower concentrations of building blocks are at the top of the image and the higher concentrations at the bottom. The present building blocks formed a continuous gradient on the derivatized slide. The cholera toxin bound in increasing amounts to the higher concentration portions of the gradient (FIG. 80).

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted, adapted and configured, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1. A building block gradient comprising:
a support; and
a portion of the support comprising at least one building block;
the building block being coupled to the support;
the building block forming a gradient.

2. The gradient of claim 1, wherein the building block comprises a plurality of building blocks.

3. The gradient of claim 1, comprising:
change in concentration of the building block;
change in identity of the building block;
change in topography of the building block;
change in the mode of binding of the building block to the support; or
change in lawn or lawn modifier.

4. The gradient of claim 1, comprising:
change in charge of the building block;
change in volume of the building block,
change in lipophilicity of the building block, or
change in hydrophilicity of the building block

5. The gradient of claim 1, comprising change in a molecular descriptor for the building block.

6. The gradient of claim 1, wherein the gradient comprises 2, 3, 4, 5, or 6 different building blocks.

7. The gradient of claim 1, wherein the support comprises a solid support.

8. The gradient of claim 1, comprising a functionalized lawn coupled to the support and the building blocks coupled in spots to the lawn.

9. The gradient of claim 8, comprising a functionalized glass support.

10. The gradient of claim 1, comprising a plurality of regions on the support;
the regions comprising a plurality of building blocks;
the building blocks being coupled to the support; and
the building blocks forming a gradient in the region.

11. The gradient of claim 10, wherein the regions comprise 2, 3, 4, 5, or 6 building blocks.

12. The gradient of claim 10, wherein the support comprises a solid support.

13. The gradient of claim 12, comprising a plurality of regions on a surface of the solid support.

14. The gradient of claim 11, comprising a functionalized lawn coupled to the support and the building blocks coupled to the lawn.

15. The gradient of claim 14, comprising a functionalized glass support.

16. The gradient of claim 1, the plurality of building blocks independently comprising framework, linker, first recognition element, and second recognition element.

17. The gradient of claim 16, wherein at least one of the building blocks comprises tether.

18. The gradient of claim 16, wherein the framework comprises an amino acid.

19. The gradient of claim 18, wherein the amino acid comprises serine, threonine, or tyrosine.

20. The gradient of claim 18, wherein the amino acid comprises tyrosine.

21. The gradient of claim 16, wherein the linker has the formula (CH₂)ₙC(O)—, with n=1-16.

22. The gradient of claim 16, wherein the first recognition element and second recognition element independently are of formulas B1, B2, B3, B4, B5, B6, B7, B8, B9, A1, A2, A3, A4, A5, A6, A7, A8, or A9.

23. The gradient of claim 16, wherein the support comprises a support matrix and the support matrix comprises a lawn of amines.

24. A building block gradient comprising:
a surface; and
a region on the surface comprising at least one building block;
the building block being coupled to the support;
the building block forming a gradient.

25. A method of making a building block gradient, the method comprising:
forming a region on a solid support, the region comprising at least one building block, the building block providing a gradient;
coupling the building block to the solid support in the region.

26. The method of claim 25, comprising coupling a plurality of building blocks to the solid support in the region.

27. The method of claim 25, comprising forming:
change in concentration of the building block;
change in identity of the building block;
change in topography of the building block;
change in the mode of binding of the building block to the support; or
change in lawn or lawn modifier.

28. The gradient of claim 25, comprising forming:
change in charge of the building block;
change in volume of the building block,
change in lipophilicity of the building block, or
change in hydrophilicity of the building block

29. The gradient of claim 25, comprising forming change in a molecular descriptor for the building block.

30. The method of claim 25, further comprising mixing a plurality of activated building blocks, and employing the mixture in forming the gradient.

31. The method of claim 25, comprising applying individual activated building blocks on the support.

32. The method of claim 25, wherein the solid support comprises a glass plate or microscope slide.

33. A method of using building block gradient comprising:
contacting the building block gradient with a test ligand;
the building block gradient comprising:
a support; and
a portion of the support comprising at least one building block;
the building block being coupled to the support;
the building block forming a gradient;
monitoring the gradient for binding of the test ligand.

34. The method of claim 33, wherein the test ligand comprises a protein or proteome.

35. The method of claim 33, wherein contacting comprises flowing a composition comprising the test ligand on the building block gradient.

36. The method of claim 33, wherein contacting comprises eluting a composition comprising the test ligand across at least a portion of the building block gradient.

37. The method of claim 33, wherein the building block gradient comprises:
change in concentration of the building block;
change in identity of the building block;
change in topography of the building block;
change in the mode of binding of the building block to the support; or
change in lawn or lawn modifier.

38. The gradient of claim 33, wherein the building block gradient comprises:
change in charge of the building block;
change in volume of the building block,
change in lipophilicity of the building block, or
change in hydrophilicity of the building block

39. The gradient of claim 33, wherein the building block gradient comprises change in a molecular descriptor for the building block.
